(19) United States
(12) Patent Application Publication
(10) Pub. No.: US 2021/0078988 A1
(43) Pub. Date: Mar. 18, 2021

(54) MODULATORS OF INDOLEAMINE 2,3-DIOXYGENASE

(71) Applicant: GLAXOSMITHKLINE INTELLECTUAL PROPERTY DEVELOPMENT LIMITED, Brentford, Middlesex (GB)

(72) Inventors: Ghotas EVINDAR, Cambridge, MA (US); Wieslaw Mieczyslaw KAZMIERSKI, Research Triangle Park, NC (US); John Franklin MILLER, Research Triangle Park, NC (US); Vicente SAMANO, Research Triangle Park, NC (US); Lita SUWANDI, Research Triangle Park, NC (US); David TEMELKOFF, Research Triangle Park, NC (US); Yoshiaki WASHIO, Stevenage, Hertfordshire (GB); Bing XIA, Cambridge, MA (US)

(21) Appl. No.: 16/618,830

(22) PCT Filed: Jun. 27, 2018
(86) PCT No.: PCT/IB2018/054761

§ 371 Date: Dec. 3, 2019

Related U.S. Application Data
(60) Provisional application No. 62/525,912, filed on Jun. 28, 2017.

(51) Int. Cl. C07D 413/14; C07D 409/12; C07D 211/26; C07D 409/14; C07D 401/06; C07D 413/06; C07D 413/12; C07D 401/12; C07D 405/12; C07D 417/12; C07D 211/96

(52) U.S. Cl. CPC C07D 413/14; C07D 409/12; C07D 211/26; C07D 409/14; C07D 401/06; C07D 211/96; C07D 413/12; C07D 401/12; C07D 405/12; C07D 417/12; C07D 413/06

(57) ABSTRACT

Provided are IDO inhibitor compounds of Formula I and pharmaceutically acceptable salts thereof, their pharmaceutical compositions, their methods of preparation, and methods for their use in the prevention and/or treatment of diseases.

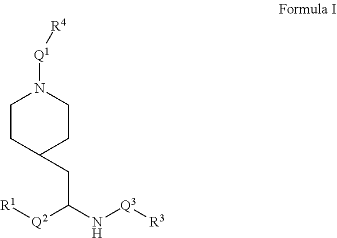

### FIELD OF THE INVENTION

Compounds, methods and pharmaceutical compositions for the prevention and/or treatment of HIV; including the prevention of the progression of AIDS and general immunosuppression, by administering certain indoleamine 2,3-dioxygenase compounds in therapeutically effective amounts are disclosed. Methods for preparing such compounds and methods of using the compounds and pharmaceutical compositions thereof are also disclosed.

### BACKGROUND OF THE INVENTION

Indoleamine-2,3-dioxygenase 1 (IDO1) is a heme-containing enzyme that catalyzes the oxidation of the indole ring of tryptophan to produce N-formyl kynurenine, which is rapidly and constitutively converted to kynurenine (Kyn) and a series of downstream metabolites. IDO1 is the rate limiting step of this kynurenine pathway of tryptophan metabolism and expression of IDO1 is inducible in the context of inflammation. Stimuli that induce IDO1 include viral or bacterial products, or inflammatory cytokines associated with infection, tumors, or sterile tissue damage. Kyn and several downstream metabolites are immunosuppressive: Kyn is antiproliferative and proapoptotic to T cells and NK cells (Munn, Shafizadeh et al. 1999, Frumento, Rotondo et al. 2002) while metabolites such as 3-hydroxy anthranilic acid (3-HAA) or the 3-HAA oxidative dimerization product cinnabarinic acid (CA) inhibit phagocyte function (Sekkai, Guittet et al. 1997), and induce the differentiation of immunosuppressive regulatory T cells (Treg) while inhibiting the differentiation of gut-protective IL-17 or IL-22-producing CD4+ T cells (Th17 and Th22)(Favre, Mold et al. 2010). IDO1 induction, among other mechanisms, is likely important in limiting immunopathology during active immune responses, in promoting the resolution of immune responses, and in promoting fetal tolerance. However in chronic settings, such as cancer, or chronic viral or bacterial infection, IDO1 activity prevents clearance of tumor or pathogen and if activity is systemic, IDO1 activity may result in systemic immune dysfunction (Boasso and Shearer 2008, Li, Huang et al. 2012). In addition to these immunomodulatory effects, metabolites of IDO1 such as Kyn and quinolinic acid are also known to be neurotoxic and are observed to be elevated in several conditions of neurological dysfunction and depression. As such, IDO1 is a therapeutic target for inhibition in a broad array of indications, such as to promote tumor clearance, enable clearance of intractable viral or bacterial infections, decrease systemic immune dysfunction manifest as persistent inflammation during HIV infection or immunosuppression during sepsis, and prevent or reverse neurological conditions.

### IDO1 and Persistent Inflammation in HIV Infection:

Despite the success of antiretroviral therapy (ART) in suppressing HIV replication and decreasing the incidence of AIDS-related conditions, HIV-infected patients on ART have a higher incidence of non-AIDS morbidities and mortality than their uninfected peers.

These non-AIDS conditions include cancer, cardiovascular disease, osteoporosis, liver disease, kidney disease, frailty, and neurocognitive dysfunction (Deeks 2011). Several studies indicate that non-AIDS morbidity/mortality is associated with persistent inflammation, which remains elevated in HIV-infected patients on ART as compared to peers (Deeks 2011). As such, it is hypothesized that persistent inflammation and immune dysfunction despite virologic suppression with ART is a cause of these non-AIDS-defining events (NADEs).

HIV infects and kills CD4+ T cells, with particular preference for cells like those CD4+ T cells that reside in the lymphoid tissues of the mucosal surfaces (Mattapallil, Douek et al. 2005). The loss of these cells combined with the inflammatory response to infection result in a perturbed relationship between the host and all pathogens, including HIV itself, but extending to pre-existing or acquired viral infections, fungal infections, and resident bacteria in the skin and mucosal surfaces. This dysfunctional host:pathogen relationship results in the over-reaction of the host to what would typically be minor problems as well as permitting the outgrowth of pathogens among the microbiota. The dysfunctional host:pathogen interaction therefore results in increased inflammation, which in turn leads to deeper dysfunction, driving a vicious cycle. As inflammation is thought to drive non-AIDS morbidity/mortality, the mechanisms governing the altered host:pathogen interaction are therapeutic targets.

IDO1 expression and activity are increased during untreated and treated HIV infection as well as in primate models of SIV infection (Boasso, Vaccari et al. 2007, Favre, Lederer et al. 2009, Byakwaga, Boum et al. 2014, Hunt, Sinclair et al. 2014, Tenorio, Zheng et al. 2014). IDO1 activity, as indicated by the ratio of plasma levels of enzyme substrate and product (Kyn/Tryp or K:T ratio), is associated with other markers of inflammation and is one of the strongest predictors of non-AIDS morbidity/mortality (Byakwaga, Boum et al. 2014, Hunt, Sinclair et al. 2014, Tenorio, Zheng et al. 2014). In addition, features consistent with the expected impact of increased IDO1 activity on the immune system are major features of HIV and SIV induced immune dysfunction, such as decreased T cell proliferative response to antigen and imbalance of Treg:Th17 in systemic and intestinal compartments (Favre, Lederer et al. 2009, Favre, Mold et al. 2010). As such, we and others hypothesize that IDO1 plays a role in driving the vicious cycle of immune dysfunction and inflammation associated with non-AIDS morbidity/mortality. Thus, we propose that inhibiting IDO1 will reduce inflammation and decrease the risk of NADEs in ART-suppressed HIV-infected persons.

### IDO1 and Persistent Inflammation Beyond HIV

As described above, inflammation associated with treated chronic HIV infection is a likely driver of multiple end organ diseases [Deeks 2011]. However, these end organ diseases are not unique to HIV infection and are in fact the common diseases of aging that occur at earlier ages in the HIV-infected population. In the uninfected general population inflammation of unknown etiology is a major correlate of morbidity and mortality [Pinti, 2016 #88]. Indeed many of the markers of inflammation are shared, such as IL-6 and CRP. If, as hypothesized above, IDO1 contributes to persistent inflammation in the HIV-infected population by inducing immune dysfunction in the GI tract or systemic tissues, then IDO1 may also contribute to inflammation and therefore end organ diseases in the broader population. These inflammation associated end organ diseases are exemplified by cardiovascular diseases, metabolic syndrome, liver disease (NAFLD, NASH), kidney disease, osteoporosis, and neurocognitive impairment. Indeed, the IDO1 pathway has links in the literature to liver disease (Vivoli abstracts at Italian Assoc. for the Study of the Liver Conference 2015], diabetes [Baban, 2010 #89], chronic kidney disease [Schefold, 2009 #90], cardiovascular disease [Mangge, 2014 #92; Mangge, 2014 #91], as well as general aging and all cause mortality [Pertovaara, 2006 #93]. As such, inhibition of IDO1 may have application in decreasing inflammation in the general population to decrease the incidence of specific end organ diseases associated with inflammation and aging.

### IDO1 and Oncology

IDO expression can be detected in a number of human cancers (for example; melanoma, pancreatic, ovarian, AML, CRC, prostate and endometrial) and correlates with poor prognosis (Munn 2011). Multiple immunosuppressive roles have been ascribed to the action of IDO, including the induction of Treg differentiation and hyper-activation, suppression of Teff immune response, and decreased DC function, all of which impair immune recognition and promote tumor growth (Munn 2011). IDO expression in human brain tumors is correlated with reduced survival. Orthotropic and transgenic glioma mouse models demonstrate a correlation between reduced IDO expression and reduced Treg infiltration and a increased long term survival (Wainwright, Balyasnikova et al. 2012). In human melanoma a high proportion of tumors (33 of 36 cases) displayed elevated IDO suggesting an important role in establishing an immunosuppressive tumor microenvironment (TME) characterized by the expansion, activation and recruitment of MDSCs in a Treg-dependent manner (Holmgaard, Zamarin et al. 2015). Additionally, host IDO expressing immune cells have been identified in the draining lymph nodes and in the tumors themselves (Mellor and Munn 2004). Hence, both tumor and host-derived IDO are believed to contribute to the immune suppressed state of the TME.

The inhibition of IDO was one of the first small molecule drug strategies proposed for re-establishment of an immunogenic response to cancer (Mellor and Munn 2004). The d-enantiomer of 1-methyl tryptophan (D-1MTor indoximod) was the first IDO inhibitor to enter clinical trials. While this compound clearly does inhibit the activity of IDO, it is a very weak inhibitor of the isolated enzyme and the in vivo mechanism(s) of action for this compound are still being elucidated. Investigators at Incyte optimized a hit compound obtained from a screening process into a potent and selective inhibitor with sufficient oral exposure to demonstrate a delay in tumor growth in a mouse melanoma model (Yue, Douty et al. 2009). Further development of this series led to INCB204360 which is a highly selective for inhibition of IDO-1 over IDO-2 and TDO in cell lines transiently transfected with either human or mouse enzymes (Liu, Shin et al. 2010). Similar potency was seen for cell lines and primary human tumors which endogenously express IDO1 (IC50s˜3-20 nM). When tested in co-culture of DCs and naïve CD4⁺CD25⁻ T cells, INCB204360 blocked the conversion of these T cells into CD4⁺FoxP3⁺ Tregs. Finally, when tested in a syngeneic model (PANO2 pancreatic cells) in immunocompetent mice, orally dosed INCB204360 provided a significant dose-dependent inhibition of tumor growth, but was without effect against the same tumor implanted in immune-deficient mice. Additional studies by the same investigators have shown a correlation of the inhibition of IDO1 with the suppression of systemic kynurenine levels and inhibition of tumor growth in an additional syngeneic tumor model in immunocompetent mice. Based upon these preclinical studies, INCB24360 entered clinical trials for the treatment of metastatic melanoma (Beatty, O'Dwyer et al. 2013).

In light of the importance of the catabolism of tryptophan in the maintenance of immune suppression, it is not surprising that overexpression of a second tryptophan metabolizing enzyme, TDO2, by multiple solid tumors (for example, bladder and liver carcinomas, melanomas) has also been detected. A survey of 104 human cell lines revealed 20/104 with TDO expression, 17/104 with IDO1 and 16/104 expressing both (Pilotte, Larrieu et al. 2012). Similar to the inhibition of IDO1, the selective inhibition of TDO2 is effective in reversing immune resistance in tumors overexpressing TDO2 (Pilotte, Larrieu et al. 2012). These results support TDO2 inhibition and/or dual TDO2/IDO1 inhibition as a viable therapeutic strategy to improve immune function.

Multiple pre-clinical studies have demonstrated significant, even synergistic, value in combining IDO-1 inhibitors in combination with T cell checkpoint modulating mAbs to CTLA-4, PD-1, and GITR. In each case, both efficacy and related PD aspects of improved immune activity/function were observed in these studies across a variety of murine models (Balachandran, Cavnar et al. 2011, Holmgaard, Zamarin et al. 2013, M. Mautino 2014, Wainwright, Chang et al. 2014). The Incyte IDO1 inhibitor (INCB204360, epacadostat) has been clinically tested in combination with a CTLA4 blocker (ipilimumab), but it is unclear that an effective dose was achieved due to dose-limited adverse events seen with the combination. In contrast recently released data for an on-going trial combining epacadostat with Merck's PD-1 mAb (pembrolizumab) demonstrated improved tolerability of the combination allowing for higher doses of the IDO1 inhibitor. There have been several clinical responses across various tumor types which is encouraging. However, it is not yet known if this combination is an improvement over the single agent activity of pembrolizumab (Gangadhar, Hamid et al. 2015). Similarly, Roche/Genentech are advancing NGL919/GDC-0919 in combination with both mAbs for PD-L1 (MPDL3280A, Atezo) and OX-40 following the recent completion of a phase 1a safety and PK/PD study in patients with advanced tumors.

### IDO1 and Chronic Infections

IDO1 activity generates kynurenine pathway metabolites such as Kyn and 3-HAA that impair at least T cell, NK cell, and macrophage activity (Munn, Shafizadeh et al. 1999, Frumento, Rotondo et al. 2002) (Sekkai, Guittet et al. 1997, Favre, Mold et al. 2010). Kyn levels or the Kyn/Tryp ratio are elevated in the setting of chronic HIV infection (Byakwaga, Boum et al. 2014, Hunt, Sinclair et al. 2014, Tenorio, Zheng et al. 2014), HBV infection (Chen, Li et al. 2009), HCV infection (Larrea, Riezu-Boj et al. 2007, Asghar, Ashiq et al. 2015), and TB infection (Suzuki, Suda et al. 2012) and are associated with antigen-specific T cell dysfunction (Boasso, Herbeuval et al. 2007, Boasso, Hardy et al. 2008, Loughman and Hunstad 2012, Ito, Ando et al. 2014, Lepiller, Soulier et al. 2015). As such, it is thought that in these cases of chronic infection, IDO1-mediated inhibition of the pathogen-specific T cell response plays a role in the persistence of infection, and that inhibition of IDO1 may have a benefit in promoting clearance and resolution of infection.

### IDO1 and Sepsis

IDO1 expression and activity are observed to be elevated during sepsis and the degree of Kyn or Kyn/Tryp elevation corresponded to increased disease severity, including mortality (Tattevin, Monnier et al. 2010, Darcy, Davis et al. 2011). In animal models, blockade of IDO1 or IDO1 genetic knockouts protected mice from lethal doses of LPS or from mortality in the cecal ligation/puncture model (Jung, Lee et al. 2009, Hoshi, Osawa et al. 2014). Sepsis is characterized by an immunosuppressive phase in severe cases (Hotchkiss, Monneret et al. 2013), potentially indicating a role for IDO1 as a mediator of immune dysfunction, and indicating that pharmacologic inhibition of IDO1 may provide a clinical benefit in sepsis.

### IDO1 and Neurological Disorders

In addition to immunologic settings, IDO1 activity is also linked to disease in neurological settings (reviewed in Lovelace Neuropharmacology 2016(Lovelace, Varney et al. 2016)). Kynurenine pathway metabolites such as 3-hydroxykynurenine and quinolinic acid are neurotoxic, but are balanced by alternative metabolites kynurenic acid or picolinic acid, which are neuroprotective. Neurodegenerative and psychiatric disorders in which kynurenine pathway metabolites have been demonstrated to be associated with disease include multiple sclerosis, motor neuron disorders such as amyotrophic lateral sclerosis, Huntington's disease, Parkinson's disease, Alzheimer's disease, major depressive disorder, schizophrenia, anorexia (Lovelace, Varney et al. 2016). Animal models of neurological disease have shown some impact of weak IDO1 inhibitors such as 1-methyltryptophan on disease, indicating that IDO1 inhibition may provide clinical benefit in prevention or treatment of neurological and psychiatric disorders.

It would therefore be an advance in the art to discover IDO inhibitors that effective the balance of the aforementioned properties as a disease modifying therapy in chronic HIV infections to decrease the incidence of non-AIDS morbidity/mortality; and/or a disease modifying therapy to prevent mortality in sepsis; and/or an immunotherapy to enhance the immune response to HIV, HBV, HCV and other chronic viral infections, chronic bacterial infections, chronic fungal infections, and to tumors; and/or for the treatment of depression or other neurological/neuropsychiatric disorders.
Asghar, K., M. T. Ashiq, B. Zulfiqar, A. Mahroo, K. Nasir and S. Murad (2015). “Indoleamine 2,3-dioxygenase expression and activity in patients with hepatitis C virus-induced liver cirrhosis.” Exp Ther Med 9(3): 901-904.
Balachandran, V. P., M. J. Cavnar, S. Zeng, Z. M. Bamboat, L. M. Ocuin, H. Obaid, E. C. Sorenson, R. Popow, C. Ariyan, F. Rossi, P. Besmer, T. Guo, C. R. Antonescu, T. Taguchi, J. Yuan, J. D. Wolchok, J. P. Allison and R. P. Dematteo (2011). “Imatinib potentiates antitumor T cell responses in gastrointestinal stromal tumor through the inhibition of Ido.” Nature Medicine 17(9): 1094-1100.
Beatty, G. L., P. J. O'Dwyer, J. Clark, J. G. Shi, R. C. Newton, R. Schaub, J. Maleski, L. Leopold and T. Gajewski (2013). “Phase I study of the safety, pharmacokinetics (PK), and pharmacodynamics (PD) of the oral inhibitor of indoleamine 2,3-dioxygenase (IDO1) INCB024360 in patients (pts) with advanced malignancies.” ASCO Meeting Abstracts 31(15_suppl): 3025.
Boasso, A., A. W. Hardy, S. A. Anderson, M. J. Dolan and G. M. Shearer (2008). “HIV-induced type I interferon and tryptophan catabolism drive T cell dysfunction despite phenotypic activation.” PLoS One 3(8): e2961.
Boasso, A., J. P. Herbeuval, A. W. Hardy, S. A. Anderson, M. J. Dolan, D. Fuchs and G. M. Shearer (2007). “HIV inhibits CD4+ T-cell proliferation by inducing indoleamine 2,3-dioxygenase in plasmacytoid dendritic cells.” Blood 109(8): 3351-3359.
Boasso, A. and G. M. Shearer (2008). “Chronic innate immune activation as a cause of HIV-1 immunopathogenesis.” Clin Immunol 126(3): 235-242.
Boasso, A., M. Vaccari, A. Hryniewicz, D. Fuchs, J. Nacsa, V. Cecchinato, J. Andersson, G. Franchini, G. M. Shearer and C. Chougnet (2007). “Regulatory T-cell markers, indoleamine 2,3-dioxygenase, and virus levels in spleen and gut during progressive simian immunodeficiency virus infection.” J Virol 81(21): 11593-11603.
Byakwaga, H., Y. Bourn, 2nd, Y. Huang, C. Muzoora, A. Kembabazi, S. D. Weiser, J. Bennett, H. Cao, J. E. Haberer, S. G. Deeks, D. R. Bangsberg, J. M. McCune, J. N. Martin and P. W. Hunt (2014). “The kynurenine pathway of tryptophan catabolism, CD4+ T-cell recovery, and mortality among HIV-infected Ugandans initiating antiretroviral therapy.” J Infect Dis 210(3): 383-391.
Chen, Y. B., S. D. Li, Y. P. He, X. J. Shi, Y. Chen and J. P. Gong (2009). “Immunosuppressive effect of IDO on T cells in patients with chronic hepatitis B*.” Hepatol Res 39(5): 463-468.
Darcy, C. J., J. S. Davis, T. Woodberry, Y. R. McNeil, D. P. Stephens, T. W. Yeo and N. M. Anstey (2011). “An observational cohort study of the kynurenine to tryptophan ratio in sepsis: association with impaired immune and microvascular function.” PLoS One 6(6): e21185.
Deeks, S. G. (2011). “HIV infection, inflammation, immunosenescence, and aging.” Annu Rev Med 62: 141-155.
Favre, D., S. Lederer, B. Kanwar, Z. M. Ma, S. Proll, Z. Kasakow, J. Mold, L. Swainson, J. D. Barbour, C. R. Baskin, R. Palermo, I. Pandrea, C. J. Miller, M. G. Katze and J. M. McCune (2009). “Critical loss of the balance between Th17 and T regulatory cell populations in pathogenic SIV infection.” PLoS Pathog 5(2): e1000295.
Favre, D., J. Mold, P. W. Hunt, B. Kanwar, P. Loke, L. Seu, J. D. Barbour, M. M. Lowe, A. Jayawardene, F. Aweeka, Y. Huang, D. C. Douek, J. M. Brenchley, J. N. Martin, F. M. Hecht, S. G. Deeks and J. M. McCune (2010). “Tryptophan catabolism by indoleamine 2,3-dioxygenase 1 alters the balance of TH17 to regulatory T cells in HIV disease.” Sci Transl Med 2(32): 32ra36.
Frumento, G., R. Rotondo, M. Tonetti, G. Damonte, U. Benatti and G. B. Ferrara (2002). “Tryptophan-derived catabolites are responsible for inhibition of T and natural killer cell proliferation induced by indoleamine 2,3-dioxygenase.” J Exp Med 196(4): 459-468.
Gangadhar, T., O. Hamid, D. Smith, T. Bauer, J. Wasser, J. Luke, A. Balmanoukian, D. Kaufman, Y. Zhao, J. Maleski, L. Leopold and T. Gajewski (2015). “Preliminary results from a Phase 1/11 study of epacadostat (incb024360) in combination with pembrolizumab in patients with selected advanced cancers.” Journal for ImmunoTherapy of Cancer 3(Suppl 2): 07.
Holmgaard, R. B., D. Zamarin, Y. Li, B. Gasmi, D. H. Munn, J. P. Allison, T. Merghoub and J. D. Wolchok (2015). “Tumor-Expressed IDO Recruits and Activates MDSCs in a Treg-Dependent Manner.” Cell Reports 13(2): 412-424.
Holmgaard, R. B., D. Zamarin, D. H. Munn, J. D. Wolchok and J. P. Allison (2013). “Indoleamine 2,3-dioxygenase is a critical resistance mechanism in antitumor T cell immunotherapy targeting CTLA-4.” Journal of Experimental Medicine 210(7): 1389-1402.
Hoshi, M., Y. Osawa, H. Ito, H. Ohtaki, T. Ando, M. Takamatsu, A. Hara, K. Saito and M. Seishima (2014). “Blockade of indoleamine 2,3-dioxygenase reduces mortality from peritonitis and sepsis in mice by regulating functions of CD11b+ peritoneal cells.” Infect Immun 82(11): 4487-4495.
Hotchkiss, R. S., G. Monneret and D. Payen (2013). “Sepsis-induced immunosuppression: from cellular dysfunctions to immunotherapy.” Nat Rev Immunol 13(12): 862-874.
Hunt, P. W., E. Sinclair, B. Rodriguez, C. Shive, B. Clagett, N. Funderburg, J. Robinson, Y. Huang, L. Epling, J. N. Martin, S. G. Deeks, C. L. Meinert, M. L. Van Natta, D. A. Jabs and M. M. Lederman (2014). “Gut epithelial barrier dysfunction and innate immune activation predict mortality in treated HIV infection.” J Infect Dis 210(8): 1228-1238.
Ito, H., T. Ando, K. Ando, T. Ishikawa, K. Saito, H. Moriwaki and M. Seishima (2014). “Induction of hepatitis B virus surface antigen-specific cytotoxic T lymphocytes can be up-regulated by the inhibition of indoleamine 2, 3-dioxygenase activity.” Immunology 142(4): 614-623.
Jung, I. D., M. G. Lee, J. H. Chang, J. S. Lee, Y. I. Jeong, C. M. Lee, W. S. Park, J. Han, S. K. Seo, S. Y. Lee and Y. M. Park (2009). “Blockade of indoleamine 2,3-dioxygenase protects mice against lipopolysaccharide-induced endotoxin shock.” J Immunol 182(5): 3146-3154.
Larrea, E., J. I. Riezu-Boj, L. Gil-Guerrero, N. Casares, R. Aldabe, P. Sarobe, M. P. Civeira, J. L. Heeney, C. Rollier, B. Verstrepen, T. Wakita, F. Borras-Cuesta, J. J. Lasarte and J. Prieto (2007). “Upregulation of indoleamine 2,3-dioxygenase in hepatitis C virus infection.” J Virol 81(7): 3662-3666.
Lepiller, Q., E. Soulier, Q. Li, M. Lambotin, J. Barths, D. Fuchs, F. Stoll-Keller, T. J. Liang and H. Barth (2015). “Antiviral and Immunoregulatory Effects of Indoleamine-2,3-Dioxygenase in Hepatitis C Virus Infection.” J Innate Immun 7(5): 530-544.
Li, L., L. Huang, H. P. Lemos, M. Mautino and A. L. Mellor (2012). “Altered tryptophan metabolism as a paradigm for good and bad aspects of immune privilege in chronic inflammatory diseases.” Front Immunol 3: 109.
Liu, X., N. Shin, H. K. Koblish, G. Yang, Q. Wang, K. Wang, L. Leffet, M. J. Hansbury, B. Thomas, M. Rupar, P. Waeltz, K. J. Bowman, P. Polam, R. B. Sparks, E. W. Yue, Y. Li, R. Wynn, J. S. Fridman, T. C. Burn, A. P. Combs, R. C. Newton and P. A. Scherle (2010). “Selective inhibition of IDO1 effectively regulates mediators of antitumor immunity.” Blood 115(17): 3520-3530.
Loughman, J. A. and D. A. Hunstad (2012). “Induction of indoleamine 2,3-dioxygenase by uropathogenic bacteria attenuates innate responses to epithelial infection.” J Infect Dis 205(12):1830-1839.
Lovelace, M. D., B. Varney, G. Sundaram, M. J. Lennon, C. K. Lim, K. Jacobs, G. J. Guillemin and B. J. Brew (2016). “Recent evidence for an expanded role of the kynurenine pathway of tryptophan metabolism in neurological diseases.” Neuropharmacoloqy.
M. Mautino, C. J. L., N. Vahanian, J. Adams, C. Van Allen, M. D. Sharma, T. S. Johnson and D. H. Munn (2014). “Synergistic antitumor effects of combinatorial immune checkpoint inhibition with anti-PD-1/PD-L antibodies and the IDO pathway inhibitors NLG919 and indoximod in the context of active immunotherapy.” April 2014 AACR Meeting Poster #5023.
Mattapallil, J. J., D. C. Douek, B. Hill, Y. Nishimura, M. Martin and M. Roederer (2005). “Massive infection and loss of memory CD4+ T cells in multiple tissues during acute SIV infection.” Nature 434(7037): 1093-1097.
Mellor, A. L. and D. H. Munn (2004). “IDO expression by dendritic cells: Tolerance and tryptophan catabolism.” Nature Reviews Immunology 4(10): 762-774.
Munn, D. H. (2011). “Indoleamine 2,3-dioxygenase, Tregs and cancer.” Current Medicinal Chemistry 18(15): 2240-2246.
Munn, D. H., E. Shafizadeh, J. T. Attwood, I. Bondarev, A. Pashine and A. L. Mellor (1999). “Inhibition of T cell proliferation by macrophage tryptophan catabolism.” J Exp Med 189(9):1363-1372.
Pilotte, L., P. Larrieu, V. Stroobant, D. Colau, E. Dolučić, R. Frédérick, E. De Plaen, C. Uyttenhove, J. Wouters, B. Masereel and B. J. Van Den Eynde (2012). “Reversal of tumoral immune resistance by inhibition of tryptophan 2,3-dioxygenase.” Proceedings of the National Academy of Sciences of the United States of America 109(7): 2497-2502.
Sekkai, D., O. Guittet, G. Lemaire, J. P. Tenu and M. Lepoivre (1997). “Inhibition of nitric oxide synthase expression and activity in macrophages by 3-hydroxyanthranilic acid, a tryptophan metabolite.” Arch Biochem Biophys 340(1): 117-123.
Suzuki, Y., T. Suda, K. Asada, S. Miwa, M. Suzuki, M. Fujie, K. Furuhashi, Y. Nakamura, N. Inui, T. Shirai, H. Hayakawa, H. Nakamura and K. Chida (2012). “Serum indoleamine 2,3-dioxygenase activity predicts prognosis of pulmonary tuberculosis.” Clin Vaccine Immunol 19(3): 436-442.
Tattevin, P., D. Monnier, O. Tribut, J. Dulong, N. Bescher, F. Mourcin, F. Uhel, Y. Le Tulzo and K. Tarte (2010). “Enhanced indoleamine 2,3-dioxygenase activity in patients with severe sepsis and septic shock.” J Infect Dis 201(6): 956-966.
Tenorio, A. R., Y. Zheng, R. J. Bosch, S. Krishnan, B. Rodriguez, P. W. Hunt, J. Plants, A. Seth, C. C. Wilson, S. G. Deeks, M. M. Lederman and A. L. Landay (2014). “Soluble markers of inflammation and coagulation but not T-cell activation predict non-AIDS-defining morbid events during suppressive antiretroviral treatment.” J Infect Dis 210(8): 1248-1259.
Wainwright, D. A., I. V. Balyasnikova, A. L. Chang, A. U. Ahmed, K.-S. Moon, B. Auffinger, A. L. Tobias, Y. Han and M. S. Lesniak (2012). “IDO Expression in Brain Tumors Increases the Recruitment of Regulatory T Cells and Negatively Impacts Survival.” Clinical Cancer Research 18(22): 6110-6121.
Wainwright, D. A., A. L. Chang, M. Dey, I. V. Balyasnikova, C. K. Kim, A. Tobias, Y. Cheng, J. W. Kim, J. Qiao, L. Zhang, Y. Han and M. S. Lesniak (2014). “Durable therapeutic efficacy utilizing combinatorial blockade against IDO, CTLA-4, and PD-L1 in mice with brain tumors.” Clinical Cancer Research 20(20): 5290-5301.
Yue, E. W., B. Douty, B. Wayland, M. Bower, X. Liu, L. Leffet, Q. Wang, K. J. Bowman, M. J. Hansbury, C. Liu, M. Wei, Y. Li, R. Wynn, T. C. Burn, H. K. Koblish, J. S. Fridman, B. Metcalf, P. A. Scherle and A. P. Combs (2009). “Discovery of potent competitive inhibitors of indoleamine 2,3-dioxygenase with in vivo pharmacodynamic activity and efficacy in a mouse melanoma model.” Journal of Medicinal Chemistry 52(23): 7364-7367.

### SUMMARY OF THE INVENTION

Briefly, in one aspect, the present invention discloses compounds of Formula I

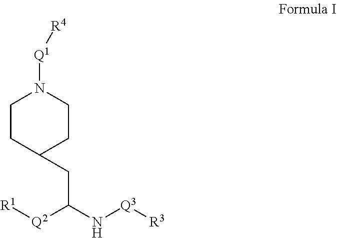

or a pharmaceutically acceptable salt thereof wherein:

Q¹ is C(O)O, C(O)CF₂, C(O)NH, SO₂, C(O), or a bond (i.e. is absent);

Q² is C₁₋₄alkyl, C₁₋₃alkylNHC₁₋₃alkyl, or a bond (i.e. is absent);

Q³ is C(O), C(O)NH, or a bond (i.e. is absent);

R¹ is C₁₋₆alkyl, C₂₋₄alkenyl, C₃₋₇cycloalkyl, C₅₋₉aryl, C₅₋₉heteroaryl, or a 5 to 9 membered heterocycle; wherein R¹ is optionally substituted with a substituent selected from C₁₋₆alkyl, OC₁₋₃alkyl, OC₃₋₆cycloalkyl, oxo, and N(R²)₂ wherein each R² is independently H, C₁₋₆alkyl, C₃₋₇cycloalkyl, C₁₋₃alkylOC₁₋₃alkyl, —OC₁₋₃alkylOC₁₋₃alkyl C₃₋₆cycloalkyl, —CH₂phenyl, or OCH₂phenyl;

R³ is C₅₋₉aryl, C₅₋₉heteroaryl, C₁₋₆alkyl, C₃₋₆cycloalkyl, or C₇₋₁₀bicycloalkyl, wherein R³ is optionally substituted with 1 or 2 substituents selected from halogen, C₁₋₆alkyl, C₁₋₃ fluoroalkyl, C₃₋₆cycloalkyl, OC₁₋₃alkyl, SC₁₋₃alkyl, C₂₋₄alkenyl, C₂₋₄alkynyl, OC₂₋₄alkyny, phenyl, and CN;

R⁴ is C₅₋₉aryl, C₁₋₆alkyl, C₁₋₃fluoroalkyl, C₃₋₆cycloalkyl, C₂₋₄alkenyl, C₂₋₄alkynyl, or C₃₋₆ether;

and wherein each aryl and heteroaryl includes bicycles and wherein each heteroaryl, and heterocycle contains from 1 to 3 heteroatoms selected from O, N, and S.

In another aspect, the present invention discloses pharmaceutical compositions comprising a compound of Formula I or a pharmaceutically acceptable salt thereof.

In another aspect, the present invention provides a compound of Formula I or a pharmaceutically acceptable salt thereof for use in therapy.

In another aspect, the present invention provides a compound of Formula I or a pharmaceutically acceptable salt thereof for use in treating diseases or conditions that would benefit from inhibition of IDO.

In another aspect, the present invention provides use of a compound of Formula I or a pharmaceutically acceptable salt thereof in the manufacture of a medicament for use in treating diseases or conditions that would benefit from inhibition of IDO.

In another aspect, the present invention discloses a method for treating a viral infection in a patient mediated at least in part by a virus in the retrovirus family of viruses, comprising administering to said patient a composition comprising a compound of Formula I, or a pharmaceutically acceptable salt thereof. In some embodiments, the viral infection is mediated by the HIV virus.

In another aspect, a particular embodiment of the present invention provides a method of treating a subject infected with HIV comprising administering to the subject a therapeutically effective amount of a compound of Formula I, or a pharmaceutically acceptable salt thereof.

In yet another aspect, a particular embodiment of the present invention provides a method of inhibiting progression of HIV infection in a subject at risk for infection with HIV comprising administering to the subject a therapeutically effective amount of a compound of Formula I, or a pharmaceutically acceptable salt thereof. Those and other embodiments are further described in the text that follows.

### DETAILED DESCRIPTION OF REPRESENTATIVE EMBODIMENTS

Preferably Q¹ is C(O)O, C(O)CF₂, C(O)NH, SO₂, or C(O).

Preferably Q² is absent.

Preferably Q³ is C(O).

Preferably R¹ is phenyl, a pyridine, an oxadiazole, oxo substituted oxadiazole, C₁₋₆ alkyl, C₃₋₇cycloalkyl, C₂₋₄alkenyl, or a 5 or 6-membered heterocycle containing one or two heteroatoms selected from O and N, wherein R¹ is optionally substituted with a substituent selected from C₁₋₆alkyl, OC₁₋₃alkyl, OC₃₋₆cycloalkyl, and N(R²)₂ wherein each R² is independently H, C₁₋₆alkyl, C₃₋₇cycloalkyl C₁₋₃alkylOC₁₋₃alkyl, —OC₁₋₃alkylOC₁₋₃alkyl C₃₋₆cycloalkyl, —CH₂phenyl, or OCH₂phenyl. More preferably, R¹ is phenyl, a pyridine, an oxadiazole, C₁₋₆alkyl, C₃₋₇cycloalkyl, or C₂₋₄alkylenyl, wherein R¹ is optionally substituted with a substituent selected from C₁₋₆alkyl, OC₁₋₃alkyl, and N(R²)₂ wherein each R² is independently C₁₋₆alkyl, or C₃₋₆cycloalkyl.

Preferably R³ is thiophene, phenyl, pyridyl, benzoxazole, oxazole, C₁₋₆alkyl, C₃₋₆cycloalkyl, or C₇₋₁₀bicycloalkyl, wherein R³ is optionally substituted with 1 or 2 substituents selected from halogen, C₁₋₃alkyl, C₁₋₃fluoroalkyl, OC₁₋₃alkyl, SC₁₋₃alkyl, C₂₋₄alkenyl, C₂₋₄alkynyl, and OC₂₋₄alkynyl. More preferably R³ is thiophene or phenyl optionally substituted with 1 or 2 substituents selected from halogen, C₁₋₃alkyl, and C₂₋₃alkynyl.

Preferably R⁴ is phenyl, C₁₋₆alkyl, C₁₋₃fluoroalkyl, C₃₋₆cycloalkyl, C₂₋₄alkynyl, or C₃₋₆ether. More preferably R⁴ is C₁₋₆alkyl.

Preferably the stereochemistry of the depicted carbon to which R¹-Q² is bonded is as depicted below.

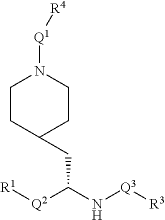

Preferred pharmaceutical compositions include unit dosage forms. Preferred unit dosage forms include tablets.

In particular, it is expected that the compounds and composition of this invention will be useful for prevention and/or treatment of HIV; including the prevention of the progression of AIDS and general immunosuppression. It is expected that in many cases such prevention and/or treatment will involve treating with the compounds of this invention in combination with at least one other drug thought to be useful for such prevention and/or treatment. For example, the IDO inhibitors of this invention may be used in combination with other immune therapies such as immune checkpoints (PD1, CTLA4, ICOS, etc.) and possibly in combination with growth factors or cytokine therapies (IL21, IL-7, etc.).

In is common practice in treatment of HIV to employ more than one effective agent. Therefore, in accordance with another embodiment of the present invention, there is provided a method for preventing or treating a viral infection in a mammal mediated at least in part by a virus in the retrovirus family of viruses which method comprises administering to a mammal, that has been diagnosed with said viral infection or is at risk of developing said viral infection, a compound as defined in Formula I, wherein said virus is an HIV virus and further comprising administration of a therapeutically effective amount of one or more agents active against an HIV virus, wherein said agent active against the HIV virus is selected from the group consisting of Nucleotide reverse transcriptase inhibitors; Non-nucleotide reverse transcriptase inhibitors; Protease inhibitors; Entry, attachment and fusion inhibitors; Integrase inhibitors; Maturation inhibitors; CXCR4 inhibitors; and CCR5 inhibitors. Examples of such additional agents are Dolutegravir, Bictegravir, and Cabotegravir.

“Pharmaceutically acceptable salt” refers to pharmaceutically acceptable salts derived from a variety of organic and inorganic counter ions well known in the art and include, by way of example only, sodium, potassium, calcium, magnesium, ammonium, and tetraalkylammonium, and when the molecule contains a basic functionality, salts of organic or inorganic acids, such as hydrochloride, hydrobromide, tartrate, mesylate, acetate, maleate, and oxalate. Suitable salts include those described in P. Heinrich Stahl, Camille G. Wermuth (Eds.), Handbook of Pharmaceutical Salts Properties, Selection, and Use; 2002.

The present invention also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or ACN are preferred.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

In one embodiment, the pharmaceutical formulation containing a compound of Formula I or a salt thereof is a formulation adapted for oral or parenteral administration. In another embodiment, the formulation is a long-acting parenteral formulation. In a further embodiment, the formulation is a nano-particle formulation.

The present invention is directed to compounds, compositions and pharmaceutical compositions that have utility as novel treatments for immunosuppresion. While not wanting to be bound by any particular theory, it is thought that the present compounds are able to inhibit the enzyme that catalyzes the oxidative pyrrole ring cleavage reaction of I-Trp to N-formylkynurenine utilizing molecular oxygen or reactive oxygen species.

Therefore, in another embodiment of the present invention, there is provided a method for the prevention and/or treatment of HIV; including the prevention of the progression of AIDS and general immunosuppression.

### EXAMPLES

Compounds of the invention can be prepared by one skilled in the art according to the following general synthetic scheme.

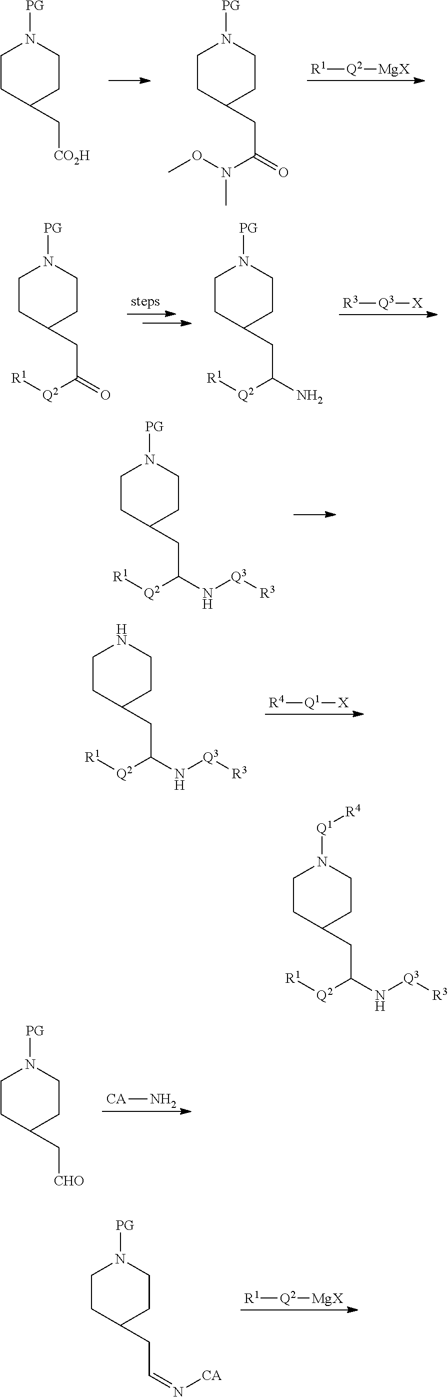

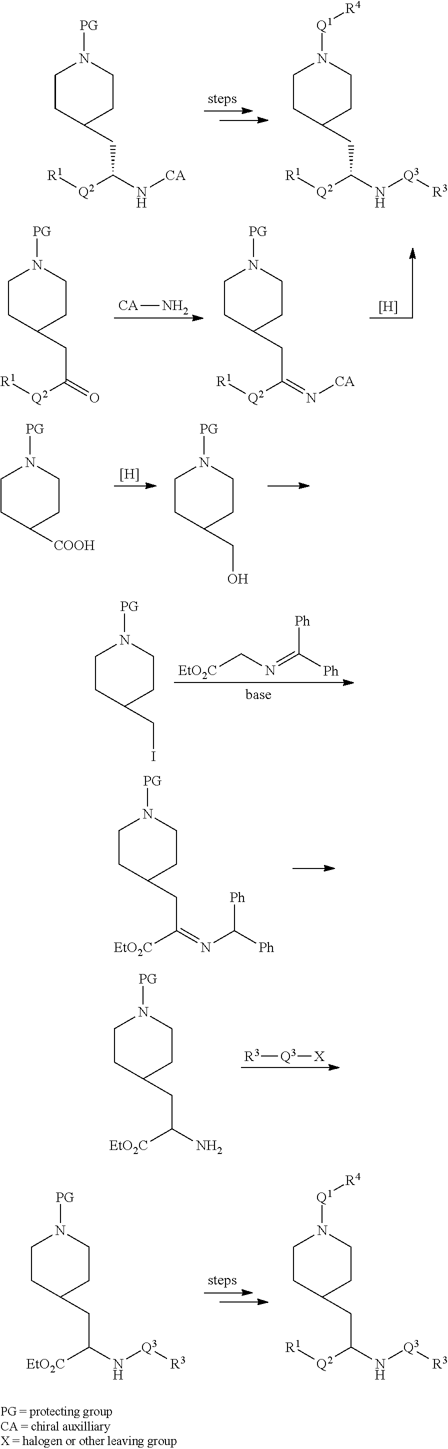

The following examples serve to more fully describe the manner of making and using the above-described invention. It is understood that these examples in no way serve to limit the true scope of the invention, but rather are presented for illustrative purposes. In the examples and the synthetic schemes below, the following abbreviations have the following meanings. If an abbreviation is not defined, it has its generally accepted meaning.

| abbreviation | meaning |
|---|---|
| Boc | tert-butoxycarbonyl |
| BOP | benzotriazole-1-yl-oxy-tris-(dimethylamino)-phosphonium |
| | hexafluorophosphate |
| ° C. | degrees Celsius |
| COMU | (1-cyano-2-ethoxy-2-oxoethylidenaminooxy)dimethyl- |
| | amino-morpholino-carbenium hexafluorophosphate |
| DBU | 1,8-diazabicyclo[5.4.0]undec-7-ene |
| DCM | dichloromethane |
| DEA | diethylamine |
| DIEA | N,N-diisopropylethylamine |
| DMAP | 4-(dimethylamino)pyridine |
| DMF | N,N-dimethylformamide |
| DMSO | dimethylsulfoxide |
| ESI | electrospray ionization |
| h or hr | hours |
| HATU | (1-[bis(dimethylamino)methylene]-1H-1,2,3-triazolo[4,5- |
| | b]pyridinium 3-oxid hexafluorophosphate) |
| HPLC | high performance liquid chromatography |
| J | coupling constant in Hz |
| LCMS | liquid chromatography - mass spectrometry |
| M | molar |
| mg | milligram |
| min | minute |
| mL | milliliters |
| mM | millimolar |
| mmol | millimole |
| μL or uL | microliters |
| μM or uM | micromolar |
| MS | mass spectrum |
| N | normal |
| NMR | nuclear magnetic resonance |
| PE | petroleum ether |
| ppm | parts per million |
| PPTS | pyridinium p-toluenesulfonate |
| RT | room temperature |
| Rf | retention factor |
| T3P | propanephosphonic acid anhydride |
| TEA | triethylamine |
| TFA | trifluoroacetic acid |
| TFAA | trifluoroacetic anhydride |
| THF | tetrahydrofuran |
| TLC | thin layer chromatography |

### Equipment Description

¹H NMR spectra were recorded on a Varian 400 spectrometer. Chemical shifts are expressed in parts per million (ppm, δ units). Coupling constants are in units of hertz (Hz). Splitting patterns describe apparent multiplicities and are designated as s (singlet), d (doublet), t (triplet), q (quartet), quint (quintet), m (multiplet), br (broad).

The analytical low-resolution mass spectra (MS) were recorded on Waters ACQUITY UPLC with SQ Detectors using a Waters BEH C18, 2.1×50 mm, 1.7 μm using a gradient elution method. Solvent A: 0.1% formic acid (FA) in water. Solvent B: 0.1% FA in acetonitrile; 30% B for 0.5 min followed by 30-100% B over 2.5 min.

### Synthesis of Amine Intermediate tert-butyl 4-(2-amino-2-phenylethyl)piperidine-1-carboxylate

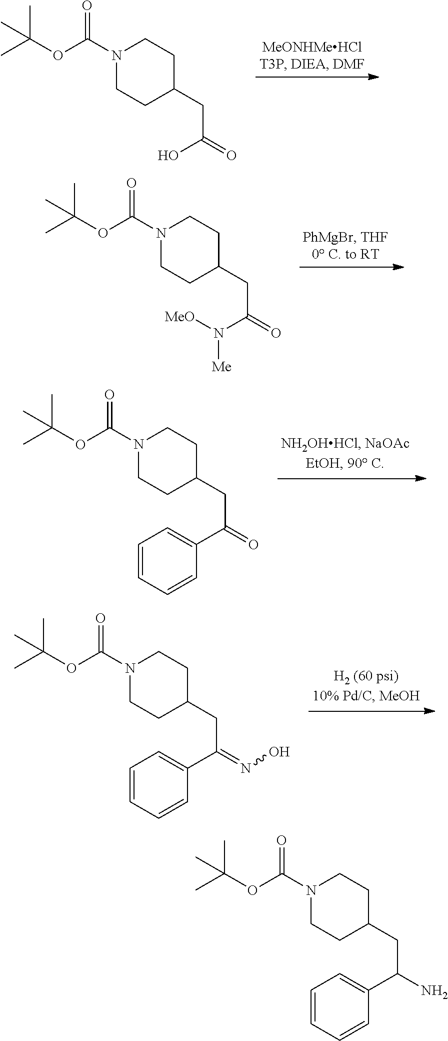 [SMILES: CC(C)(C)OC(=O)N1CCC(CC(=O)O)CC1 ; CON(C)C(=O)CC1CCN(C(=O)OC(C)(C)C)CC1 ; CC(C)(C)OC(=O)N1CCC(CC(=O)c2ccccc2)CC1 ; CC(C)(C)OC(=O)N1CCC(CC(=NO)c2ccccc2)CC1 ; CC(C)(C)OC(=O)N1CCC(CC(N)c2ccccc2)CC1]

### Step 1: Preparation of tert-butyl 4-(2-(methoxy(methyl)amino)-2-oxoethyl)piperidine-1-carboxylate

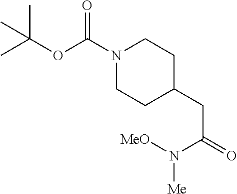 [SMILES: CON(C)C(=O)CC1CCN(C(=O)OC(C)(C)C)CC1]

To a stirred solution of 2-(1-(tert-butoxycarbonyl)piperidin-4-yl)acetic acid (10.0 g, 41.1 mmol), N,O-dimethylhydroxylamine hydrochloride (4.21 g, 43.2 mmol), and DIEA (21.5 mL, 123 mmol) in DMF (75 mL) at 0° C. was added 50% T3P/EtOAc (34.0 g, 53.4 mmol) by slow addition over 3 minutes. The resulting solution was stirred at 0° C. After 2.5 hours the solution was partitioned between EtOAc and water and the phases separated. The aqueous phase was extracted with one additional portion of EtOAc. The combined EtOAc solutions were washed with 10% aqueous citric acid (2×), saturated aqueous NaHCO₃ (2×), dried over Na₂SO₄, and concentrated to dryness at reduced pressure to give the title compound as a colorless oil (9.16 g, 78% yield). LCMS (ESI) m/z calcd for C₁₄H₂₆N₂O₄: 286.2. Found: 287.4 (M+1)⁺. ¹H NMR (400 MHz, CDCl₃) 4.07 (d, J=13.2 Hz, 2H), 3.67 (s, 3H), 3.18 (s, 3H), 2.73 (t, J=12.9 Hz, 2H), 2.35 (d, J=6.4 Hz, 2H), 1.93-2.10 (m, 1H), 1.71 (d, J=12.8 Hz, 2H), 1.45 (s, 9H), 1.08-1.21 (m, 2H).

### Step 2: Preparation of tert-butyl 4-(2-oxo-2-phenylethyl)piperidine-1-carboxylate

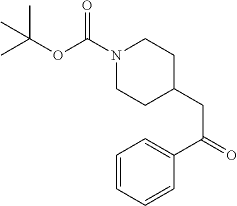 [SMILES: CC(C)(C)OC(=O)N1CCC(CC(=O)c2ccccc2)CC1]

To a stirred solution of tert-butyl 4-(2-(methoxy(methyl)amino)-2-oxoethyl)piperidine-1-carboxylate (9.13 g, 31.9 mmol) in anhydrous THE (106 mL) at 0° C. was added 1M PhMgBr (38.3 mL, 38.3 mmol) by dropwise addition. After 10 minutes the solution was allowed to warm to RT. After 2 hours the solution was quenched by addition of saturated NH₄Cl. The resulting mixture was partitioned between water and EtOAc and the phases separated. The aqueous phase was extracted with EtOAc (2×). The combined EtOAc solutions were washed with water (1×), saturated brine (1×), dried over Na₂SO₄ and concentrated at reduced. The residue was subjected to flash chromatography (silica gel, 0-50% EtOAc/hexanes, gradient elution) to afford the title compound as a white crystalline solid (9.04 g, 93% yield). LCMS (ESI) m/z calcd for C₁₈H₂₅NO₃: 303.2. Found: 204.2 (M+1-Boc)⁺. ¹H NMR (400 MHz, CDCl₃) δ 7.95 (d, J=7.3 Hz, 2H), 7.53-7.61 (m, 1H), 7.41-7.52 (m, 2H), 4.09 (d, J=13.2 Hz, 2H), 2.90 (d, J=6.6 Hz, 2H), 2.70-2.81 (m, 2H), 2.06-2.25 (m, 1H), 1.75 (d, J=12.6 Hz, 2H), 1.46 (s, 9H), 1.13-1.27 (m, 2H).

### Step 3: Preparation of tert-butyl 4-(2-(hydroxyimino)-2-phenylethyl)piperidine-1-carboxylate

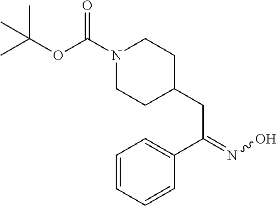 [SMILES: CC(C)(C)OC(=O)N1CCC(CC(=NO)c2ccccc2)CC1]

A solution of tert-butyl 4-(2-oxo-2-phenylethyl)piperidine-1-carboxylate (3.40 g, 11.2 mmol), NaOAc (4.60 g, 56.0 mmol), and hydroxylamine hydrochloride (1.56 g, 22.4 mmol) in 2:1 EtOH/H₂O (80 mL) was stirred at 90° C. for 3 hours and then cooled to RT. The solution was partitioned between EtOAc and water and the phases separated. The aqueous phase was extracted with two additional portions of EtOAc. The combined EtOAc solutions were washed with brine (1×), dried over Na₂SO₄ and concentrated at reduced pressure to give the title compound as a white crystalline solid (3.52 g, 99% yield). LCMS (ESI) m/z calcd for C₁₈H₂₆N₂O₃: 318.2. Found: 319.4 (M+1)⁺. ¹H NMR (400 MHz, CDCl₃) δ 7.56-7.65 (m, 2H), 7.33-7.46 (m, 3H), 4.04 (br s, 2H), 2.81 (d, J=7.3 Hz, 2H), 2.63 (t, J=12.1 Hz, 2H), 1.75-1.87 (m, 1H), 1.64 (d, J=13.0 Hz, 2H), 1.45 (s, 9H), 1.14-1.33 (m, 2H).

### Step 4: Preparation of tert-butyl 4-(2-amino-2-phenylethyl)piperidine-1-carboxylate

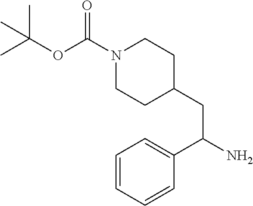 [SMILES: CC(C)(C)OC(=O)N1CCC(CC(N)c2ccccc2)CC1]

A solution of tert-butyl 4-(2-(hydroxyimino)-2-phenylethyl)piperidine-1-carboxylate (3.52 g, 11.1 mmol) in MeOH (75 mL) was subjected to hydrogenation at 60 psi in the presence of 10% Pd/C (0.25 g). After 18 hours the reaction vessel was purged with nitrogen, catalyst removed by filtration, and the filtrate concentrated at reduced pressure to give the title compound as a colorless oil (3.35 g, 100%). LCMS (ESI) m/z calcd for C₁₈H₂₈N₂O₂: 304.2. Found: 305.4 (M+1)⁺. ¹H NMR (400 MHz, CDCl₃) δ 7.20-7.38 (m, 5H), 3.87-4.18 (m, 3H), 2.55-2.70 (m, 2H), 1.51-1.88 (m, 6H), 1.34-1.49 (m, 10H), 0.99-1.21 (m, 2H).

### Example 1: tert-butyl 4-(2-(5-ethylthiophene-2-carboxamido)-2-phenylethyl)piperidine-1-carboxylate

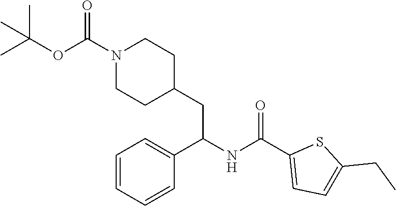 [SMILES: CCc1ccc(C(=O)NC(CC2CCN(C(=O)OC(C)(C)C)CC2)c2ccccc2)s1]

To a stirred solution of tert-butyl 4-(2-amino-2-phenylethyl)piperidine-1-carboxylate (50.0 mg, 0.164 mmol), 5-ethylthiophene-2-carboxylic acid (28.2 mg, 0.181 mmol), and DIEA (86 uL mL, 0.49 mmol) in DMF (2 mL) was added HATU (94 mg, 0.25 mmol). The resulting solution was stirred at RT. After 18 hours the solution was treated with 2M ammonia/MeOH (3 mL). After stirring at RT for an additional 1 hour, the solution was partitioned between EtOAc and brine and the phases separated. The EtOAc solution was washed with 10% aqueous citric acid (2×), saturated aqueous NaHCO₃ (2×), dried over Na₂SO₄, and concentrated to dryness at reduced pressure. The residue was subjected to flash chromatography (silica gel, 0-100% EtOAc/hexanes, gradient elution) to afford the title compound as a white solid (54 mg, 74% yield). LCMS (ESI) m/z calcd for C₂₅H₃₄N₂O₃S: 442.2. Found: 465.3 (M+Na)⁺. ¹H NMR (400 MHz, CDCl₃) δ 7.28-7.45 (m, 6H), 6.80 (d, J=3.5 Hz, 1H), 6.03 (d, J=8.3 Hz, 1H), 5.22-5.37 (m, 1H), 4.09 (br s, 2H), 2.89 (q, J=7.4 Hz, 2H), 2.67 (t, J=12.5 Hz, 2H), 1.69-1.98 (m, 4H), 1.42-1.55 (m, 10H), 1.35 (t, J=7.6 Hz, 3H), 1.10-1.30 (m, 2H).

### Example 2: tert-butyl 4-(2-(5-bromothiophene-2-carboxamido)-2-phenylethyl)piperidine-1-carboxylate

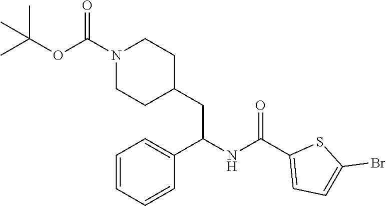 [SMILES: CC(C)(C)OC(=O)N1CCC(CC(NC(=O)c2ccc(Br)s2)c2ccccc2)CC1]

The title compound was prepared in 77% yield from tert-butyl 4-(2-amino-2-phenylethyl)piperidine-1-carboxylate and 5-bromothiophene-2-carboxylic acid as described herein for the preparation of tert-butyl 4-(2-(5-ethylthiophene-2-carboxamido)-2-phenylethyl)piperidine-1-carboxylate. LCMS (ESI) m/z calcd for C₂₃H₂₉BrN₂O₃S: 492.1. Found: 493.2 (M+1)⁺. ¹H NMR (400 MHz, CDCl₃) δ 7.27-7.40 (m, 5H), 7.20 (d, J=3.9 Hz, 1H), 7.01 (d, J=3.9 Hz, 1H), 6.01 (d, J=8.2 Hz, 1H), 5.22 (q, J=8.2 Hz, 1H), 3.95-4.10 (m, 2H), 2.61 (t, J=12.3 Hz, 2H), 1.65-1.94 (m, 4H), 1.35-1.50 (m, 10H), 1.01-1.30 (m, 2H).

### Example 3: tert-butyl 4-(2-(5-chlorothiophene-2-carboxamido)-2-phenylethyl)piperidine-1-carboxylate

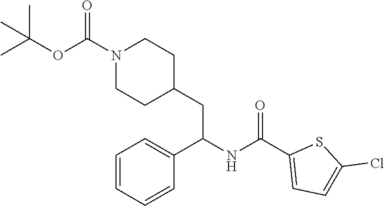 [SMILES: CC(C)(C)OC(=O)N1CCC(CC(NC(=O)c2ccc(Cl)s2)c2ccccc2)CC1]

To a stirred solution of tert-butyl 4-(2-amino-2-phenylethyl)piperidine-1-carboxylate (0.785 g, 2.58 mmol) in DMF (30 mL) was added COMU (1.63 g, 3.81 mmol) followed by DIEA (1.36 mL, 7.79 mmol) and then 5-chlorothiophene-2-carboxylic acid (0.544 g, 3.35 mmol). After stirring at RT for 2 hours, the solution was quenched with water and partitioned between DCM and saturated aqueous Na₂CO₃. The phases were separated and the aqueous phase extracted with DCM (2×). The combined DCM solutions were concentrated to dryness at reduced pressure and the residue purified by reverse phase HPLC (C18, MeCN/water with ammonium carbonate modifier) to afford the title compound. LCMS (ESI) m/z calcd for C₂₃H₂₉ClN₂O₃S: 448.1. Found: 449.1 (M+1)⁺. ¹H NMR (400 MHz, CDCl₃) δ 7.22-7.44 (m, 6H), 6.90 (d, J=4.0 Hz, 1H), 6.00 (d, J=8.1 Hz, 1H), 5.25 (q, J=8.1 Hz, 1H), 4.07 (br s, 2H), 2.58-2.71 (m, 2H), 1.69-1.96 (m, 4H), 1.38-1.53 (m, 10H), 1.07-1.34 (m, 2H).

### Example 4: tert-butyl 4-(2-(5-ethynylthiophene-2-carboxamido)-2-phenylethyl)piperidine-1-carboxylate

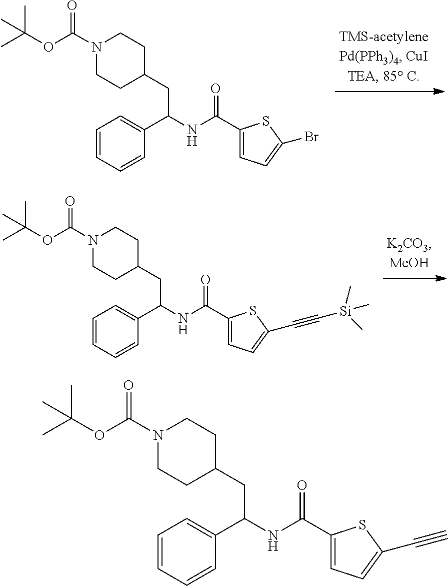

### Step 1: Preparation of tert-butyl 4-(2-phenyl-2-(5-((trimethylsilyl)ethynyl)thiophene-2-carboxamido)ethyl)piperidine-1-carboxylate

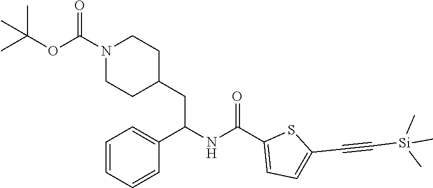 [SMILES: CC(C)(C)OC(=O)N1CCC(CC(NC(=O)c2ccc(C#C[Si](C)(C)C)s2)c2ccccc2)CC1]

A stirred solution of tert-butyl 4-(2-(5-bromothiophene-2-carboxamido)-2-phenylethyl)piperidine-1-carboxylate (76 mg, 0.15 mmol), tetrakis(triphenylphosphine)palladium(0) (18 mg, 0.015 mmol), and copper(I) iodide (2.9 mg, 0.015 mmol) in THE (3 mL) was sparged with nitrogen for 5 minutes, and then treated with TEA (0.107 mL, 0.770 mmol) followed by TMS-acetylene (0.107 mL, 0.770 mmol). The resulting solution was heated to 85° C. in a sealed vessel. After 30 minutes LCMS indicated complete reaction. The mixture was cooled to RT, filtered to removed solids, and the filtrate concentrated to dryness at reduced pressure. The residue was subjected to flash chromatography (silica gel, 0-100% EtOAc/hexanes, gradient elution) to afford the title compound as a light yellow foam (68 mg, 86%). LCMS (ESI) m/z calcd for C₂₈H₃₈N₂O₃SSi: 510.2. Found: 511.4 (M+1)⁺.

### Step 2: Preparation of tert-butyl 4-(2-(5-ethynylthiophene-2-carboxamido)-2-phenylethyl)piperidine-1-carboxylate

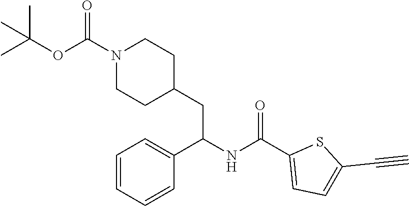 [SMILES: C#Cc1ccc(C(=O)NC(CC2CCN(C(=O)OC(C)(C)C)CC2)c2ccccc2)s1]

To a stirred solution of tert-butyl 4-(2-phenyl-2-(5-((trimethylsilyl)ethynyl)thiophene-2-carboxamido)ethyl)piperidine-1-carboxylate (68 mg, 0.13 mmol) in MeOH (3 mL) was added K₂CO₃ (92 mg, 0.67 mmol). The resulting mixture was stirred at RT. After 1 hour the mixture was partitioned between EtOAc and 10% aqueous citric acid and the phases separated. The EtOAc solution was washed with saturated aqueous NaHCO₃ (2×), dried over Na₂SO₄ and concentrated at reduced pressure. The residue was subjected to flash chromatography (silica gel, 0-100% EtOAc/hexanes, gradient elution) to afford the title compound as a white solid (42 mg, 72% yield). LCMS (ESI) m/z calcd for C₂₅H₃₀N₂O₃S: 438.2. Found: 439.3 (M+1)⁺. ¹H NMR (400 MHz, CDCl₃) δ 7.26-7.40 (m, 6H), 7.18 (d, J=3.9 Hz, 1H), 6.09 (d, J=8.2 Hz, 1H), 5.24 (q, J=7.8 Hz, 1H), 4.04 (br s, 2H), 3.42 (s, 1H), 2.54-2.68 (m, 2H), 1.66-1.95 (m, 4H), 1.37-1.50 (m, 10H), 1.02-1.30 (m, 2H).

### Example 5: tert-butyl 4-(2-(4-ethynylbenzamido)-2-phenylethyl)piperidine-1-carboxylate

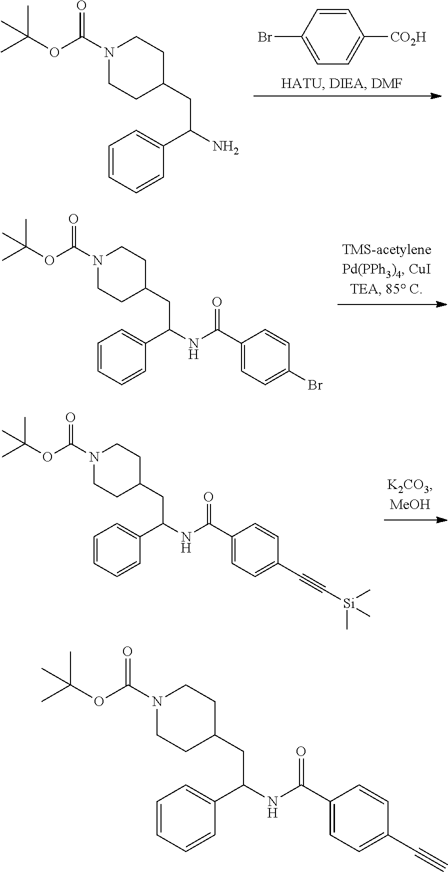

### Step 1: Preparation of tert-butyl 4-(2-(4-bromobenzamido)-2-phenylethyl)piperidine-1-carboxylate

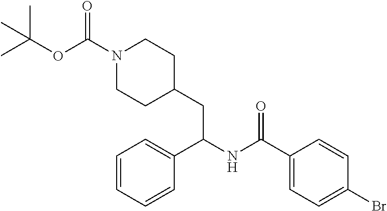 [SMILES: CC(C)(C)OC(=O)N1CCC(CC(NC(=O)c2ccc(Br)cc2)c2ccccc2)CC1]

The title compound was prepared in 85% yield from tert-butyl 4-(2-amino-2-phenylethyl)piperidine-1-carboxylate and 4-bromobenzoic acid as described herein for the preparation of tert-butyl 4-(2-(5-ethylthiophene-2-carboxamido)-2-phenylethyl)piperidine-1-carboxylate. LCMS (ESI) m/z calcd for C₂₅H₃₁BrN₂O₃: 486.2. Found: 487.2 (M+1)⁺. ¹H NMR (400 MHz, CDCl₃) δ 7.52-7.65 (m, 4H), 7.23-7.40 (m, 5H), 6.21 (d, J=8.2 Hz, 1H), 5.23-5.33 (m, 1H), 3.97-4.10 (m, 2H), 2.53-2.67 (m, 2H), 1.66-1.95 (m, 4H), 1.36-1.48 (m, 10H), 1.07-1.29 (m, 2H).

### Steps 2 and 3: Preparation of tert-butyl 4-(2-(4-ethynylbenzamido)-2-phenylethyl)piperidine-1-carboxylate

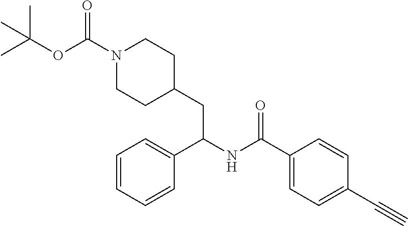 [SMILES: C#Cc1ccc(C(=O)NC(CC2CCN(C(=O)OC(C)(C)C)CC2)c2ccccc2)cc1]

The title compound was prepared in two steps in 31% overall yield from tert-butyl 4-(2-(4-bromobenzamido)-2-phenylethyl)piperidine-1-carboxylate as described herein for the preparation of tert-butyl 4-(2-(5-ethynylthiophene-2-carboxamido)-2-phenylethyl)piperidine-1-carboxylate. LCMS (ESI) m/z calcd for C₂₇H₃₂N₂O₃: 432.2. Found: 433.3 (M+1)⁺. ¹H NMR (400 MHz, CDCl₃) δ 7.68 (d, J=8.2 Hz, 2H), 7.51 (d, J=8.2 Hz, 2H), 7.25-7.38 (m, 5H), 6.21 (d, J=7.8 Hz, 1H), 5.28 (q, J=7.8 Hz, 1H), 4.03 (br s, 2H), 3.17 (s, 1H), 2.53-2.66 (m, 2H), 1.63-1.95 (m, 4H), 1.35-1.46 (m, 10H), 0.98-1.27 (m, 2H).

### Example 6: tert-butyl 4-(2-(4-ethynyl-3-fluorobenzamido)-2-phenylethyl)piperidine-1-carboxylate

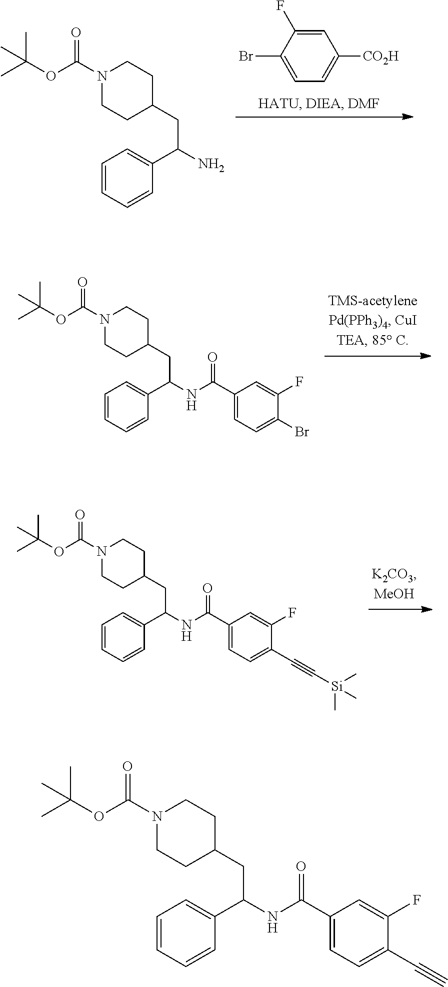

### Step 1: Preparation of tert-butyl 4-(2-(4-bromo-3-fluorobenzamido)-2-phenylethyl)piperidine-1-carboxylate

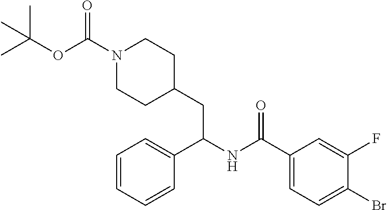 [SMILES: CC(C)(C)OC(=O)N1CCC(CC(NC(=O)c2ccc(Br)c(F)c2)c2ccccc2)CC1]

The title compound was prepared in 82% yield from tert-butyl 4-(2-amino-2-phenylethyl)piperidine-1-carboxylate and 4-bromo-3-fluorobenzoic acid as described herein for the preparation of tert-butyl 4-(2-(5-ethylthiophene-2-carboxamido)-2-phenylethyl)piperidine-1-carboxylate. LCMS (ESI) m/z calcd for C₂₅H₃₀BrFN₂O₃: 504.1. Found: 505.3 (M+1)⁺. ¹H NMR (400 MHz, CDCl₃) δ 7.60 (dd, J=8.2, 6.6 Hz, 1H), 7.52 (dd, J=9.0, 2.0 Hz, 1H), 7.27-7.41 (m, 6H), 6.24 (d, J=8.2 Hz, 1H), 5.21-5.31 (m, 1H), 3.99-4.09 (m, 2H), 2.54-2.66 (m, 2H), 1.66-1.93 (m, 4H), 1.34-1.47 (m, 10H), 1.06-1.29 (m, 2H).

### Steps 2 and 3: Preparation of tert-butyl 4-(2-(4-ethynyl-3-fluorobenzamido)-2-phenylethyl)piperidine-1-carboxylate

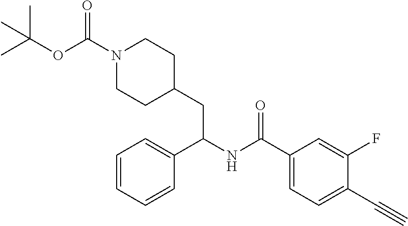 [SMILES: C#Cc1ccc(C(=O)NC(CC2CCN(C(=O)OC(C)(C)C)CC2)c2ccccc2)cc1F]

The title compound was prepared in two steps in 57% overall yield from tert-butyl 4-(2-(4-bromo-3-fluorobenzamido)-2-phenylethyl)piperidine-1-carboxylate as described herein for the preparation of tert-butyl 4-(2-(5-ethynylthiophene-2-carboxamido)-2-phenylethyl)piperidine-1-carboxylate. LCMS (ESI) m/z calcd for C₂₇H₃₁FN₂O₃: 450.2. Found: 451.3 (M+1)⁺. ¹H NMR (400 MHz, CDCl₃) δ 7.43-7.57 (m, 3H), 7.27-7.42 (m, 5H), 6.25 (d, J=8.2 Hz, 1H), 5.27 (q, J=7.8 Hz, 1H), 4.05 (br s, 2H), 3.41 (s, 1H), 2.53-2.67 (m, 2H), 1.65-1.96 (m, 4H), 1.34-1.52 (m, 10H), 1.03-1.31 (m, 2H).

### Example 7: ethyl 4-(2-(5-chlorothiophene-2-carboxamido)-2-cyclopropylethyl)piperidine-1-carboxylate

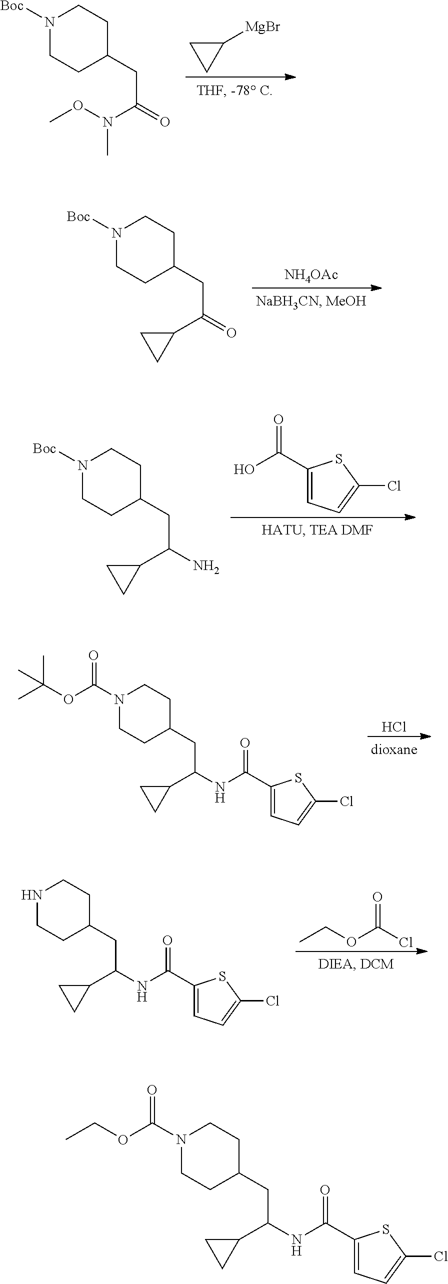

### Step 1: Preparation of tert-butyl 4-(2-cyclopropyl-2-oxoethyl)piperidine-1-carboxylate

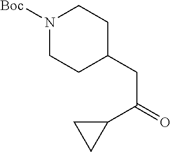 [SMILES: CC(C)(C)OC(=O)N1CCC(CC(=O)C2CC2)CC1]

To a solution of tert-butyl 4-(2-(methoxy(methyl)amino)-2-oxoethyl)piperidine-1-carboxylate (13.4 g, 46.9 mmol) in THE (200 mL) −78° C., was slowly added a solution of 1M cyclopropylmagnesium bromide in THE (141 mL, 141 mmol). After stirring at RT overnight, the reaction was quenched with saturated aqueous NH₄Cl and extracted with EtOAc. The organic layer was washed with brine, dried over Na₂SO₄, filtered and concentrated to give the crude product which was purified by flash chromatography (silica gel, 0-15% EtOAc in PE, gradient elution) to afford the title compound (9.0 g, 72% yield). LCMS (ESI) m/z calcd for C₁₅H₂₅NO₃: 267.2. Found: 268.3 (M+1)⁺.

### Step 2: Preparation of tert-butyl 4-(2-amino-2-cyclopropylethyl)piperidine-1-carboxylate

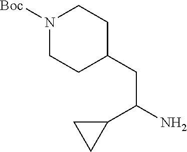 [SMILES: CC(C)(C)OC(=O)N1CCC(CC(N)C2CC2)CC1]

To a solution of tert-butyl 4-(2-cyclopropyl-2-oxoethyl)piperidine-1-carboxylate (500 mg, 1.87 mmol) in MeOH (8 mL), was added NH₄OAc (2.88 g, 37.3 mmol) and NaBH₃CN (1.18 mg, 18.7 mmol) successively. After stirring at RT overnight, the reaction was quenched with saturated aqueous NH₄Cl and extracted with EtOAc. The organic layer was washed with brine, dried over Na₂SO₄, filtered and concentrated to give the title compound (510 mg, quantitative yield), which was used in the following step without purification. LCMS (ESI) m/z calcd for C₁₅H₂₈N₂O₂: 268.2. Found: 269.4 (M+1)⁺.

### Step 3: Preparation of tert-butyl 4-(2-(5-chlorothiophene-2-carboxamido)-2-cyclopropyl ethyl)piperidine-1-carboxylate

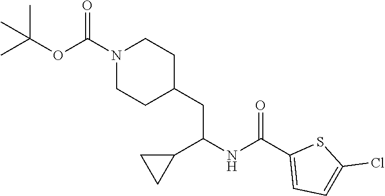 [SMILES: CC(C)(C)OC(=O)N1CCC(CC(NC(=O)c2ccc(Cl)s2)C2CC2)CC1]

To a solution of tert-butyl 4-(2-amino-2-cyclopropylethyl)piperidine-1-carboxylate (502 mg, 1.87 mmol) in DMF (8 mL), was added 5-chlorothiophene-2-carboxylic acid (365 mg, 2.24 mmol), DIEA (1.13 mL, 6.48 mmol) and HATU (853 mg, 2.24 mmol) successively. After stirring at RT for 3 hours, the reaction was diluted with water and extracted with EtOAc. The organic layer was washed with brine, dried over Na₂SO₄, filtered and concentrated to give the crude product which was purified by flash chromatography (silica gel, 0-20% EtOAc in PE, gradient elution) to afford the title compound (650 mg, 84% yield). LCMS (ESI) m/z calcd for C₂H₂₉ClN₂O₃S: 412.2. Found: 413.7 (M+1)⁺.

### Step 4: Preparation of 5-chloro-N-(1-cyclopropyl-2-(piperidin-4-yl)ethyl)thiophene-2-carboxamide hydrochloride

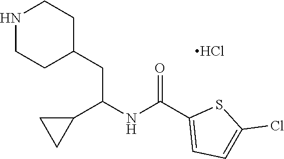 [SMILES: O=C(NC(CC1CCNCC1)C1CC1)c1ccc(Cl)s1 ; Cl]

To a solution of tert-butyl 4-(2-(5-chlorothiophene-2-carboxamido)-2-cyclopropyl ethyl)piperidine-1-carboxylate (200 mg, 0.35 mmol) in DCM (2 mL), was added 4 M HCl in dioxane (3 mL) dropwise. After stirring at RT for 2 hours, the reaction mixture was concentrated to afford the title compound (220 mg, 100% yield), which was used in the following step without purification. LCMS (ESI) m/z calcd for C₁₅H₂₁ClN₂OS: 312.1. Found: 313.7 (M+1)⁺

### Step 5: Preparation of ethyl 4-(2-(5-chlorothiophene-2-carboxamido)-2-cyclopropylethyl) piperidine-1-carboxylate

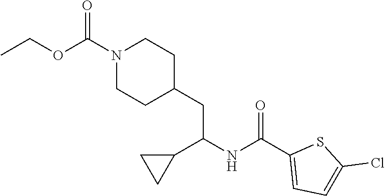 [SMILES: CCOC(=O)N1CCC(CC(NC(=O)c2ccc(Cl)s2)C2CC2)CC1]

To a solution of 5-chloro-N-(1-cyclopropyl-2-(piperidin-4-yl)ethyl)thiophene-2-carboxamide (140 mg, 0.448 mmol), DIEA (0.37 mL, 2.24 mmol) in DCM (2 mL) at 0° C., was added ethyl chloroformate (0.13 mL, 1.34 mmol) dropwise. After stirring at RT for 2 hours, the reaction was quenched with saturated aqueous NaHCO₃ solution and extracted with EtOAc. The organic layer was washed with brine, dried over Na₂SO₄, filtered and concentrated to give the crude product which was purified by flash chromatography (silica gel, 0-20% EtOAc in PE, gradient elution) to afford the title compound (93 mg, 54% yield). LCMS (ESI) m/z calcd for C₁₈H₂₅ClN₂O₃S: 384.1. Found: 385.3 (M+1)⁺. ¹H NMR (400 MHz, DMSO-d₆) δ 8.34 (d, J=8.8 Hz, 1H), 7.68 (d, J=4.1 Hz, 1H), 7.17 (d, J=4.0 Hz, 1H), 4.00 (q, J=7.1 Hz, 2H), 3.95-3.81 (m, 2H), 3.47-3.38 (m, 1H), 2.79-2.58 (m, 2H), 1.72-1.54 (m, 3H), 1.52-1.42 (m, 2H), 1.15 (t, J=7.1 Hz, 3H), 1.10-0.99 (m, 1H), 0.98-0.86 (m, 2H), 0.50-0.42 (m, 1H), 0.39-0.32 (m, 1H), 0.31-0.23 (m, 1H), 0.21-0.13 (m, 1H).

### Synthesis of Intermediate (S)-tert-butyl 4-(2-amino-2-cyclopropylethyl)piperidine-1-carboxylatehydrochloride

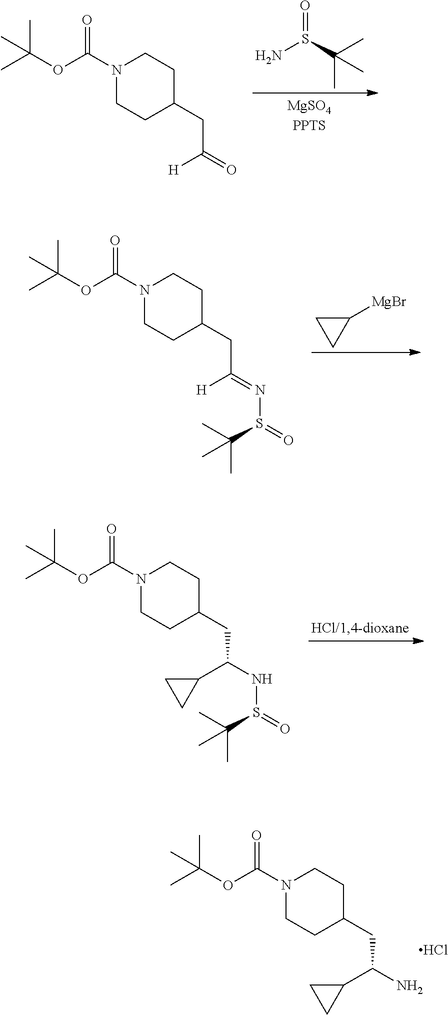

### Step 1: Preparation of (S,E)-tert-butyl 4-(2-((tert-butylsulfinyl)imino)ethyl)piperidine-1-carboxylate

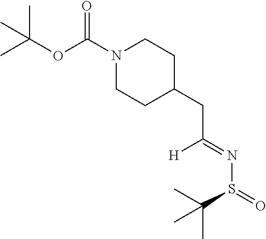 [SMILES: CC(C)(C)OC(=O)N1CCC(C/C=N/[S@@](=O)C(C)(C)C)CC1]

To a solution of (S)-2-methylpropane-2-sulfinamide (1.76 g, 14.5 mmol) in DCM (36 mL) was added PPTS (0.166 g, 0.660 mmol) and magnesium sulfate (3.97 g, 33.0 mmol) followed by N-Boc-piperidineacetaldehyde (3.00 g, 13.2 mmol) and the mixture was stirred at ambient temperature for 18 hours. The mixture was filtered and the filtrate concentrated. The material was subjected to flash chromatography (silica gel, dry loading, 0-40% EtOAc/hexanes, gradient elution) to provide the title compound (4.04 g, 93% yield) as an off-white solid. LCMS (ESI) m/z calcd for C₁₆H₃₀N₂S: 330.2. Found: 331.4 (M+1)⁺. ¹H NMR (400 MHz, CDCl₃) δ 8.05 (t, J=4.9 Hz, 1H), 4.07 (br s, 2H), 2.70 (t, J=12.1 Hz, 2H), 2.41-2.53 (m, 2H), 1.91 (ddd, J=11.0, 7.3, 3.9 Hz, 1H), 1.64-1.77 (m, 3H), 1.44 (s, 9H), 1.11-1.27 (m, 10H).

### Step 2: Preparation of tert-butyl 4-((S)-2-cyclopropyl-2-((S)-1,1-dimethylethylsulfinamido) ethyl)piperidine-1-carboxylate

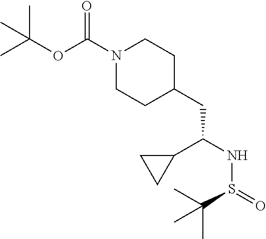 [SMILES: CC(C)(C)OC(=O)N1CCC(C[C@H](N[S@@](=O)C(C)(C)C)C2CC2)CC1]

To a solution of tert-butyl (S,E)-4-(2-((tert-butylsulfinyl)imino)ethyl)piperidine-1-carboxylate (1.60 g, 4.84 mmol) in DCM (120 mL) at ambient temperature under a nitrogen atmosphere was added dropwise in 10 minutes 0.5M cyclopropylmagnesium bromide/THF (10.7 mL, 5.33 mmol). After stirring for 1 hour, saturated NH₄C/water was added and the mixture was extracted with DCM. The organic phase was washed with water, brine, dried (Na₂SO₄), concentrated and dried in vacuo to provide a thick oil. The material was subjected to flash chromatography (silica gel, dry loading, 0-20% acetone/hexanes, gradient elution) to provide the title compound (0.88 g, 49% yield). LCMS (ESI) m/z calcd for C₁₉H₃₆N₂O₃S: 372.2. Found: 373.4 (M+1)⁺. ¹H NMR (400 MHz, CDCl₃) δ 4.07 (br s, 2H), 3.12 (br s, 1H), 2.57-2.78 (m, 2H), 2.50 (d, J=6.3 Hz, 1H), 1.52-1.76 (m, 6H), 1.44 (s, 9H), 1.21 (s, 9H), 1.02-1.15 (m, 1H), 0.77-0.93 (m, 1H), 0.54-0.67 (m, 2H), 0.42 (dd, J=9.0, 4.7 Hz, 1H), 0.19-0.31 (m, 1H).

### Step 3: Preparation of (S)-tert-butyl 4-(2-amino-2-cyclopropylethyl)piperidine-1-carboxylatehydrochloride

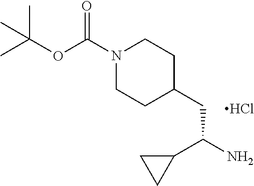 [SMILES: CC(C)(C)OC(=O)N1CCC(C[C@H](N)C2CC2)CC1 ; Cl]

To a solution of 4-((S)-2-cyclopropyl-2-((S)-1,1-dimethylethylsulfinamido) ethyl)piperidine-1-carboxylate (550 mg, 1.48 mmol) in MeOH (8.5 mL) was added 4M HCl/dioxane (0.369 mL, 1.48 mmol) and the mixture was stirred at ambient temperature for 30 minutes. The mixture was concentrated and the resulting pale yellow solid was dried in vacuo to provide the title compound (450 mg, 95% yield) as on off-white solid. LCMS (ESI) m/z calcd for C₁₅H₂₈N₂O₂: 268.2. Found: 269.4 (M+1)⁺. ¹H NMR (400 MHz, DMSO-d₆) δ 7.90 (br s, 3H), 3.89 (d, J=10.5 Hz, 2H), 2.65 (br s, 2H), 1.44-1.74 (m, 5H), 1.36 (s, 9H), 0.75-1.00 (m, 3H), 0.53-0.62 (m, 1H), 0.46-0.52 (m, 1H), 0.42 (dd, J=9.4, 4.7 Hz, 1H), 0.23-0.34 (m, 1H).

### Example 8: (S)-tert-butyl 4-(2-(5-chlorothiophene-2-carboxamido)-2-cyclopropylethyl)piperidine-1-carboxylate

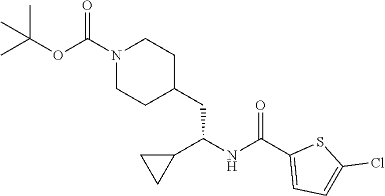 [SMILES: CC(C)(C)OC(=O)N1CCC(C[C@H](NC(=O)c2ccc(Cl)s2)C2CC2)CC1]

To a suspension of tert-butyl (S)-4-(2-amino-2-cyclopropylethyl)piperidine-1-carboxylate hydrochloride (30 mg, 0.112 mmol) in EtOAc (1.5 mL) was added 5-chlorothiophene-2-carboxylic acid (20.0 mg, 0.123 mmol), DIEA (0.078 mL, 0.45 mmol) and 50% T3P/EtOAc (78 mg, 0.123 mmol). The mixture was stirred at ambient temperature for 18 hours then diluted with EtOAc and the solution washed with water. The organic phase was dried (Na₂SO₄), concentrated and the residue subjected to flash chromatography (silica gel, dry loading, 0-30% EtOAc/hexanes, gradient elution) to provide the title compound (12 mg, 26%) as a solid foam. LCMS (ESI) m/z calcd for C₂₀H₂₉ClN₂O₃S: 412.2. Found: 413.3 (M+1)⁺. ¹H NMR (400 MHz, CDCl₃) δ 7.22 (d, J=3.9 Hz, 1H), 6.88 (d, J=3.9 Hz, 1H), 5.64 (d, J=8.9 Hz, 1H), 4.03 (d, J=12.1 Hz, 2H), 3.42-3.65 (m, 1H), 2.54-2.73 (m, 2H), 1.78 (d, J=13.3 Hz, 1H), 1.47-1.65 (m, 4H), 1.42 (s, 9H), 0.98-1.33 (m, 1H), 0.75-0.92 (m, 2H), 0.51-0.64 (m, 1H), 0.35-0.50 (m, 2H), 0.27 (dt, J=9.5, 4.8 Hz, 1H).

### Example 9: (S)-tert-butyl 4-(2-cyclopropyl-2-(5-methylthiophene-2-carboxamido)ethyl)piperidine-1-carboxylate

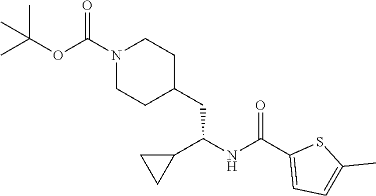 [SMILES: Cc1ccc(C(=O)N[C@@H](CC2CCN(C(=O)OC(C)(C)C)CC2)C2CC2)s1]

The title compound was prepared from tert-butyl (S)-4-(2-amino-2-cyclopropylethyl)piperidine-1-carboxylate hydrochloride and 5-methylthiophene-2-carboxylic acid as described herein for the synthesis of (S)-tert-butyl 4-(2-(5-chlorothiophene-2-carboxamido)-2-cyclopropylethyl)piperidine-1-carboxylate. LCMS (ESI) m/z calcd for C₂₁H₃₂N₂O₃S: 392.2. Found: 393.3 (M+1)⁺. ¹H NMR (400 MHz, CDCl₃) δ 7.30 (d, J=3.5 Hz, 1H), 6.74 (d, J=3.5 Hz, 1H), 5.67 (d, J=9.0 Hz, 1H), 4.04 (d, J=12.9 Hz, 2H), 3.59 (t, J=7.2 Hz, 1H), 2.64 (t, J=12.9 Hz, 2H), 2.51 (s, 3H), 1.81 (d, J=13.3 Hz, 1H), 1.50-1.66 (m, 6H), 1.44 (s, 9H), 0.77-0.91 (m, 1H), 0.50-0.61 (m, 1H), 0.37-0.50 (m, 2H), 0.28 (dt, J=9.1, 4.6 Hz, 1H).

### Example 10: (S)-tert-butyl 4-(2-cyclopropyl-2-(5-ethylthiophene-2-carboxamido)ethyl)piperidine-1-carboxylate

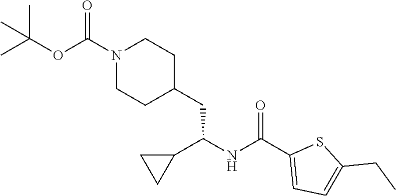 [SMILES: CCc1ccc(C(=O)N[C@@H](CC2CCN(C(=O)OC(C)(C)C)CC2)C2CC2)s1]

The title compound was prepared from tert-butyl (S)-4-(2-amino-2-cyclopropylethyl)piperidine-1-carboxylate hydrochloride and 5-ethylthiophene-2-carboxylic acid as described herein for the synthesis of (S)-tert-butyl 4-(2-(5-chlorothiophene-2-carboxamido)-2-cyclopropylethyl)piperidine-1-carboxylate. LCMS (ESI) m/z calcd for C₂₂H₃₄N₂O₃S: 406.2. Found: 407.4 (M+1)⁺. ¹H NMR (400 MHz, CDCl₃) δ 7.33 (d, J=3.5 Hz, 1H), 6.77 (d, J=3.1 Hz, 1H), 5.67 (d, J=9.0 Hz, 1H), 4.04 (d, J=12.9 Hz, 2H), 3.60 (t, J=7.2 Hz, 1H), 2.86 (q, J=7.7 Hz, 2H), 2.65 (t, J=12.9 Hz, 2H), 1.82 (d, J=12.9 Hz, 1H), 1.51-1.66 (m, 6H), 1.44 (s, 9H), 1.32 (t, J=7.6 Hz, 3H), 0.77-0.91 (m, 1H), 0.50-0.61 (m, 1H), 0.37-0.49 (m, 2H), 0.28 (dt, J=9.2, 4.8 Hz, 1H).

### Example 11: (S)-ethyl 4-(2-cyclopropyl-2-(5-methylthiophene-2-carboxamido)ethyl)piperidine-1-carboxylate

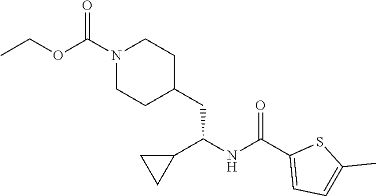 [SMILES: CCOC(=O)N1CCC(C[C@H](NC(=O)c2ccc(C)s2)C2CC2)CC1]

The title compound was prepared in two steps from (S)-tert-butyl 4-(2-cyclopropyl-2-(5-methylthiophene-2-carboxamido)ethyl)piperidine-1-carboxylate as described herein for the preparation of ethyl 4-(2-(5-chlorothiophene-2-carboxamido)-2-cyclopropylethyl)piperidine-1-carboxylate. LCMS (ESI) m/z calcd for C₁₉H₂₈N₂O₃S: 364.2. Found: 365.3 (M+1). ¹H NMR (400 MHz, CDCl₃) δ 7.30 (d, J=3.1 Hz, 1H), 6.74 (d, J=3.1 Hz, 1H), 5.66 (d, J=8.9 Hz, 1H), 4.10 (q, J=7.0 Hz, 4H), 3.50-3.67 (m, 1H), 2.70 (t, J=12.7 Hz, 2H), 2.51 (s, 3H), 1.84 (d, J=12.9 Hz, 1H), 1.51-1.67 (m, 5H), 1.24 (t, J=6.8 Hz, 3H), 0.99-1.20 (m, 1H), 0.75-0.93 (m, 1H), 0.51-0.62 (m, 1H), 0.36-0.49 (m, 2H), 0.28 (dt, J=9.1, 4.6 Hz, 1H).

### Example 12: ethyl (S)-4-(2-(5-chlorothiophene-2-carboxamido)-2-cyclopropyl ethyl)piperidine-1-carboxylate

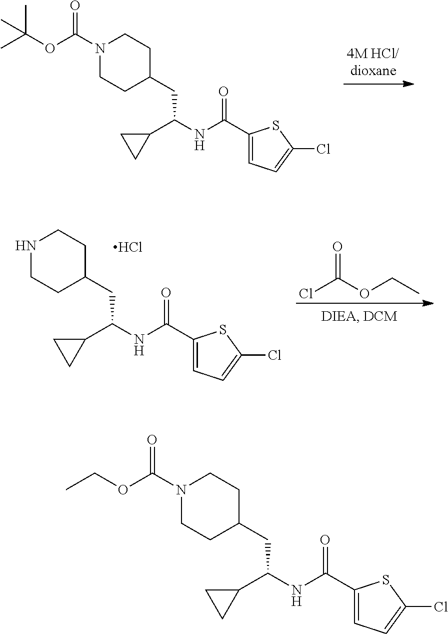

### Step 1: Preparation of (S)-5-chloro-N-(1-cyclopropyl-2-(piperidin-4-yl)ethyl)thiophene-2-carboxamide hydrochloride

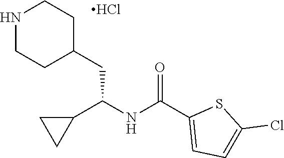 [SMILES: O=C(N[C@@H](CC1CCNCC1)C1CC1)c1ccc(Cl)s1 ; Cl]

To a solution of tert-butyl (S)-4-(2-(5-chlorothiophene-2-carboxamido)-2-cyclopropyl ethyl)piperidine-1-carboxylate (40 mg, 0.097 mmol) in DCM (0.5 mL) was added 4 M HCl in dioxane (1.0 mL). After stirred at RT for 1 hour, the reaction mixture was concentrated under vacuum to afford the title compound (35 mg, 100% yield) as an HCl salt, which was used in the following step directly. LCMS (ESI) m/z calcd for C₁₅H₂₁ClN₂OS: 312.1. Found: 313.2 (M+1)⁺.

### Step 2: Preparation of ethyl (S)-4-(2-(5-chlorothiophene-2-carboxamido)-2-cyclopropyl ethyl)piperidine-1-carboxylate

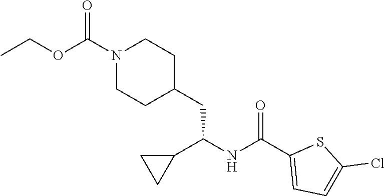 [SMILES: CCOC(=O)N1CCC(C[C@H](NC(=O)c2ccc(Cl)s2)C2CC2)CC1]

To a stirred solution of (S)-5-chloro-N-(1-cyclopropyl-2-(piperidin-4-yl)ethyl)thiophene-2-carboxamide hydrochloride (35 mg, 0.097 mmol) in DCM (1 mL) at 0° C. was added DIEA (50 mg, 0.38 mmol) followed by ethyl chloroformate (31 mg, 0.29 mmol). After stirring at RT for 2 hours, the reaction mixture was partitioned between DCM and water, and the layers were separated. The organic layer was washed with aqueous NaHCO₃, brine, and dried over Na₂SO₄. Solvent was removed under vacuum and the residue was purified by reverse phase HPLC (C18, 10-50% MeCN in water with 0.1% formic acid) to afford the title compound (16 mg, 43% yield) as a white solid. LCMS (ESI) m/z calcd for C₁₈H₂₅ClN₂O₃S: 384.1. Found: 385.2 (M+1)⁺. ¹H NMR (400 MHz, DMSO-d₆) δ 8.34 (d, J=8.9 Hz, 1H), 7.68 (d, J=4.1 Hz, 1H), 7.17 (d, J=4.0 Hz, 1H), 4.00 (q, J=7.1 Hz, 2H), 3.95-3.85 (m, 2H), 3.46-3.38 (m, 1H), 2.76-2.58 (m, 2H), 1.71-1.54 (m, 3H), 1.51-1.43 (m, 2H), 1.15 (t, J=7.1 Hz, 3H), 1.09-1.00 (m, 1H), 0.97-0.87 (m, 2H), 0.50-0.42 (m, 1H), 0.38-0.25 (m, 2H), 0.20-0.13 (m, 1H).

### Example 13: (S)-methyl 4-(2-(5-chlorothiophene-2-carboxamido)-2-cyclopropylethyl)piperidine-1-carboxylate

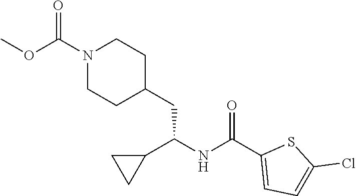 [SMILES: COC(=O)N1CCC(C[C@H](NC(=O)c2ccc(Cl)s2)C2CC2)CC1]

The title compound was prepared from (S)-5-chloro-N-(1-cyclopropyl-2-(piperidin-4-yl)ethyl)thiophene-2-carboxamide hydrochloride and methyl chloroformate in 50% yield as described herein for the synthesis of ethyl (S)-4-(2-(5-chlorothiophene-2-carboxamido)-2-cyclopropyl ethyl)piperidine-1-carboxylate. LCMS (ESI) m/z calcd for C₁₇H₂₃ClN₂O₃S: 370.1. Found: 371.3 (M+1)⁺. ¹H NMR (400 MHz, DMSO-d₆) δ 8.34 (d, J=8.8 Hz, 1H), 7.68 (d, J=4.1 Hz, 1H), 7.17 (d, J=4.0 Hz, 1H), 3.98-3.82 (m, 2H), 3.56 (s, 3H), 3.46-3.38 (m, 1H), 2.78-2.58 (m, 2H), 1.73-1.41 (m, 5H), 1.10-0.87 (m, 3H), 0.50-0.41 (m, 1H), 0.39-0.24 (m, 2H), 0.21-0.13 (m, 1H).

### Example 14: isopropyl (S)-4-(2-(5-chlorothiophene-2-carboxamido)-2-cyclopropylethyl)piperidine-1-carboxylate

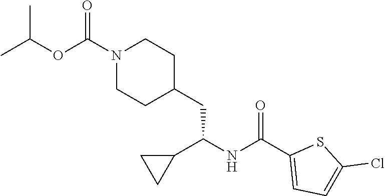 [SMILES: CC(C)OC(=O)N1CCC(C[C@H](NC(=O)c2ccc(Cl)s2)C2CC2)CC1]

The title compound was prepared from (S)-5-chloro-N-(1-cyclopropyl-2-(piperidin-4-yl)ethyl)thiophene-2-carboxamide hydrochloride and isopropyl chloroformate in 40% yield as described herein for the synthesis of ethyl (S)-4-(2-(5-chlorothiophene-2-carboxamido)-2-cyclopropyl ethyl)piperidine-1-carboxylate. LCMS (ESI) m/z calcd for C₁₉H₂₇ClN₂O₃S: 398.1. Found: 399.3 (M+1)⁺. ¹H NMR (400 MHz, DMSO-d₆) δ 8.34 (d, J=8.8 Hz, 1H), 7.68 (d, J=4.0 Hz, 1H), 7.17 (d, J=4.0 Hz, 1H), 4.78-4.67 (m, 1H), 4.00-3.81 (m, 2H), 3.47-3.37 (m, 1H), 2.79-2.58 (m, 2H), 1.71-1.43 (m, 5H), 1.16 (d, J=6.2 Hz, 6H), 1.08-0.89 (m, 3H), 0.50-0.42 (m, 1H), 0.39-0.24 (m, 2H), 0.21-0.14 (m, 1H).

### Example 15: tert-butyl 4-(2-(5-chlorothiophene-2-carboxamido)-2-(6-methoxypyridin-3-yl)ethyl)piperidine-1-carboxylate

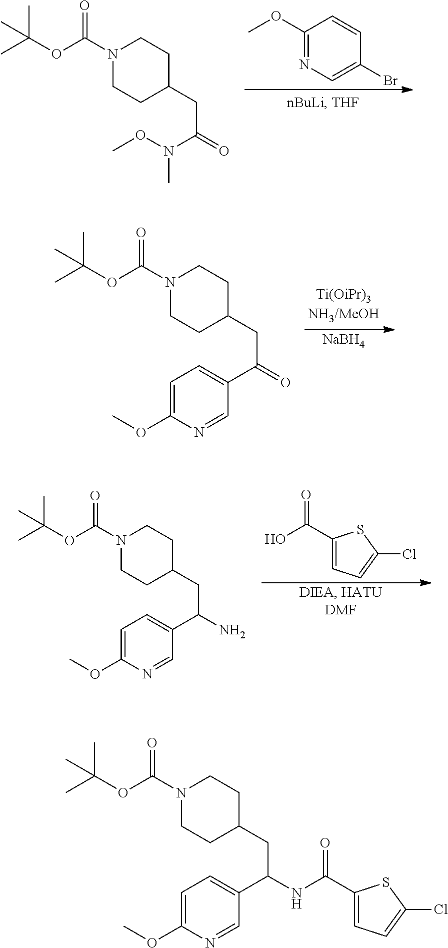

### Step 1: Preparation of tert-butyl 4-(2-(6-methoxypyridin-3-yl)-2-oxoethyl)piperidine-1-carboxylate

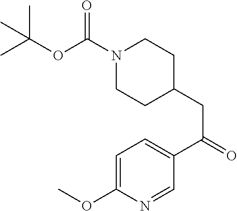 [SMILES: COc1ccc(C(=O)CC2CCN(C(=O)OC(C)(C)C)CC2)cn1]

A solution of 5-bromo-2-methoxypyridine (0.55 ml, 4.25 mmol) in THE (10 ml) was cooled to −78° C., treated dropwise with 2.5M nBuLi/hexanes (1.70 ml, 4.25 mmol), and stirred at the same temperature for 1 hour. The reaction was treated slowly with a solution of tert-butyl 4-(2-(methoxy(methyl)amino)-2-oxoethyl)piperidine-1-carboxylate (1.00 g, 3.49 mmol) in THE (10 ml), and stirred for 1 hour while letting the bath slowly warm up. The bath was removed, and the reaction was stirred at RT for 15 minutes. The mixture was quenched with saturated NH4Cl, extracted with EtOAc, washed with brine, dried over Na₂SO₄, filtered, and concentrated. Purification by flash chromatography (silica gel, 0-70% EtOAc/hexanes, gradient elution) afforded the title compound (0.94 g, 80% yield) as light yellow oil that slowly crystallized. LCMS (ESI) m/z calcd for C₁₈H₂₆N₂O₄: 334.2. Found: 357.4 (M+23)⁺. ¹H NMR (400 MHz, CDCl₃) δ 8.78 (d, J=2.2 Hz, 1H), 8.14 (dd, J=2.5, 8.7 Hz, 1H), 6.80 (d, J=8.6 Hz, 1H), 4.18-3.96 (m, 5H), 2.83 (d, J=6.8 Hz, 2H), 2.75 (t, J=12.1 Hz, 2H), 2.22-2.08 (m, 1H), 1.73 (d, J=13.0 Hz, 2H), 1.46 (s, 9H), 1.31-1.12 (m, 2H).

### Step 2: Preparation of tert-butyl 4-(2-amino-2-(6-methoxypyridin-3-yl)ethyl)piperidine-1-carboxylate formic acid salt

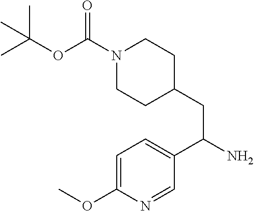 [SMILES: COc1ccc(C(N)CC2CCN(C(=O)OC(C)(C)C)CC2)cn1]

A solution of tert-butyl 4-(2-(6-methoxypyridin-3-yl)-2-oxoethyl)piperidine-1-carboxylate (0.314 g, 0.939 mmol) in 2M ammonia/EtOH (7 mL, 14.0 mmol) was treated with titanium(IV) isopropoxide (1.10 mL, 3.76 mmol) and stirred at RT in a screw cap tube. After 18 hours, the reaction was treated with additional titanium(IV) isopropoxide (0.55 mL), stirred at RT for 1 hour, and then heated at 65° C. for 1 hour. The reaction was cooled to 0° C., treated with NaBH₄ (53.3 mg, 1.41 mmol) and stirred at RT for 18 hours. The mixture was poured to aqueous NH₄OH, diluted with EtOH, and stirred for 20 minutes at RT. The suspension was filtered and washed with EtOH, and then EtOAc. The filtrate was concentrated, the residue was diluted with water, extracted with EtOAc, washed with brine, dried over Na₂SO₄, filtered, and concentrated. Purification by reverse phase flash chromatography (ISCO C18 column, 5-55% MeCN/water with 0.1% formic acid) afforded the title compound as the formic acid salt (261 mg, 73% yield) as white solid. LCMS (ESI) m/z calcd for C₁₈H₂₉N₃O₃: 335.2. Found: 336.4 (M+1)⁺. ¹H NMR (400 MHz, methanol-d₄) δ 8.50 (br s, 1H), 8.23 (d, J=2.4 Hz, 1H), 7.78 (dd, J=2.5, 8.7 Hz, 1H), 6.90 (d, J=8.8 Hz, 1H), 4.39 (dd, J=5.7, 10.1 Hz, 1H), 4.02 (t, J=14.6 Hz, 2H), 3.93 (s, 3H), 2.78-2.48 (m, 2H), 2.02-1.81 (m, 2H), 1.76 (d, J=12.6 Hz, 1H), 1.61 (d, J=13.9 Hz, 1H), 1.52-1.38 (m, 9H), 1.37-1.23 (m, 1H), 1.22-1.03 (m, 2H).

### Step 3: Preparation of tert-butyl 4-(2-(5-chlorothiophene-2-carboxamido)-2-(6-methoxypyridin-3-yl)ethyl)piperidine-1-carboxylate

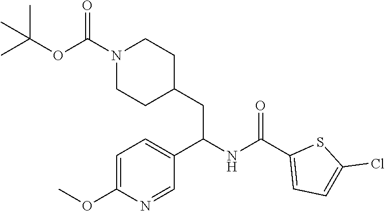 [SMILES: COc1ccc(C(CC2CCN(C(=O)OC(C)(C)C)CC2)NC(=O)c2ccc(Cl)s2)cn1]

A suspension of tert-butyl 4-(2-amino-2-(6-methoxypyridin-3-yl)ethyl)piperidine-1-carboxylate formic acid salt (30 mg, 0.079 mmol) in DMF (1 mL) was treated with 5-chlorothiophene-2-carboxylic acid (15.3 mg, 0.094 mmol), DIEA (0.048 mL, 0.275 mmol), HATU (36 mg, 0.094 mmol), and stirred at RT for 2 hours. The reaction was treated with additional DIEA (50 uL), HATU (36 mg), and stirred at RT for another 45 minutes. The mixture was diluted with water, extracted with EtOAc, washed with water, brine, dried over Na₂SO₄, filtered, and concentrated. Purification by reverse phase HPLC (C18, 15-100% MeCN/water with 0.1% formic acid) afforded the title compound (12 mg, 31% yield) as a white solid. LCMS (ESI) m/z calcd for C₂₃H₃₀ClN₃O₄S: 479.2. Found: 478.5 (M−1)⁻. ¹H NMR (400 MHz, DMSO-d₆) δ 8.84 (d, J=8.4 Hz, 1H), 8.14 (d, J=2.2 Hz, 1H), 7.79-7.65 (m, 2H), 7.19 (d, J=4.0 Hz, 1H), 6.80 (d, J=8.4 Hz, 1H), 5.08-4.94 (m, 1H), 3.97-3.85 (m, 2H), 3.82 (s, 3H), 2.74-2.55 (m, 2H), 1.92-1.76 (m, 1H), 1.72-1.57 (m, 3H), 1.49-1.32 (m, 10H), 1.13-0.92 (m, 2H).

### Example 16: tert-butyl (S)-4-(2-(5-chlorothiophene-2-carboxamido)-2-(6-methoxypyridin-3-yl)ethyl)piperidine-1-carboxylate

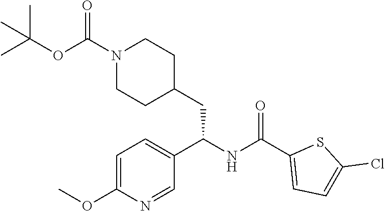 [SMILES: COc1ccc([C@H](CC2CCN(C(=O)OC(C)(C)C)CC2)NC(=O)c2ccc(Cl)s2)cn1]

The title compound (white solid) was prepared in 52% yield from tert-butyl (S)-4-(2-amino-2-(6-methoxypyridin-3-yl)ethyl)piperidine-1-carboxylate and 5-chlorothiophene-2-carboxylic acid as described herein for the preparation of tert-butyl (S)-4-(2-(4-bromobenzamido)-2-(6-methoxypyridin-3-yl)ethyl)piperidine-1-carboxylate. LCMS (ESI) m/z calcd for C₂₃H₃₀ClN₃O₄S: 479.2. Found: 480.3 (M+1)⁺. ¹H NMR (400 MHz, CDCl₃) δ 8.17 (br s, 1H), 7.56 (d, J=8.2 Hz, 1H), 7.24 (br. s., 1H), 6.89 (br s, 1H), 6.75 (d, J=8.4 Hz, 1H), 6.01 (d, J=7.5 Hz, 1H), 5.19 (q, J=7.1 Hz, 1H), 4.21-3.99 (m, 2H), 3.93 (s, 3H), 2.74-2.50 (m, 2H), 1.99-1.65 (m, 4H), 1.55-1.33 (m, 10H), 1.30-1.05 (m, 2H).

### Example 17: tert-butyl (S)-4-(2-(4-fluorobenzamido)-2-(6-methoxypyridin-3-yl)ethyl)piperidine-1-carboxylate

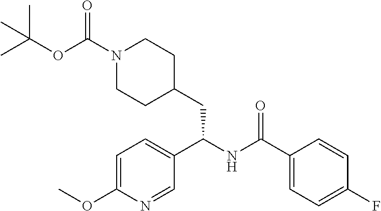 [SMILES: COc1ccc([C@H](CC2CCN(C(=O)OC(C)(C)C)CC2)NC(=O)c2ccc(F)cc2)cn1]

The title compound (white solid) was prepared in 36% yield from tert-butyl (S)-4-(2-amino-2-(6-methoxypyridin-3-yl)ethyl)piperidine-1-carboxylate and 4-fluorobenzoic acid as described herein for the preparation of tert-butyl (S)-4-(2-(4-bromobenzamido)-2-(6-methoxypyridin-3-yl)ethyl)piperidine-1-carboxylate. LCMS (ESI) m/z calcd for C₂₅H₃₂FN₃O₄: 457.2. Found: 458.4 (M+1)⁺. ¹H NMR (400 MHz, CDCl₃) δ 8.19 (brs, 1H), 7.83-7.68 (m, 2H), 7.59 (dd, J=1.7, 8.3 Hz, 1H), 7.11 (t, J=8.4 Hz, 2H), 6.75 (d, J=8.4 Hz, 1H), 6.20 (d, J=7.7 Hz, 1H), 5.25 (q, J=7.6 Hz, 1H), 4.17-3.99 (m, 2H), 3.93 (s, 3H), 2.78-2.50 (m, 2H), 1.97-1.66 (m, 4H), 1.53-1.36 (m, 10H), 1.32-1.07 (m, 2H).

### Example 18: tert-butyl 4-(2-(5-chlorothiophene-2-carboxamido)-4-methylpentyl)piperidine-1-carboxylate

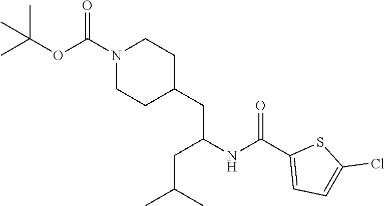 [SMILES: CC(C)CC(CC1CCN(C(=O)OC(C)(C)C)CC1)NC(=O)c1ccc(Cl)s1]

The title compound (white solid) was prepared in 4 steps from tert-butyl 4-(2-(methoxy(methyl)amino)-2-oxoethyl)piperidine-1-carboxylate and isobutylmagnesium bromide as described herein for the preparation of tert-butyl 4-(2-(5-ethylthiophene-2-carboxamido)-2-phenylethyl)piperidine-1-carboxylate. LCMS (ESI) m/z calcd for C₂₁H₃₃ClN₂O₃S: 428.2. Found: 451.3 (M+23)⁺. ¹H NMR (400 MHz, CDCl₃) δ 7.23 (d, J=3.9 Hz, 1H), 6.90 (d, J=3.9 Hz, 1H), 5.43 (d, J=9.4 Hz, 1H), 4.37-4.22 (m, 1H), 4.20-3.92 (m, 2H), 2.80-2.54 (m, 2H), 1.88 (d, J=12.5 Hz, 1H), 1.72-1.54 (m, 1H), 1.54-1.25 (m, 15H), 1.22-1.00 (m, 2H), 0.99-0.85 (m, 6H).

### Example 19: tert-butyl (S)-4-(2-(4-chlorobenzamido)-2-(6-methoxypyridin-3-yl)ethyl)piperidine-1-carboxylate

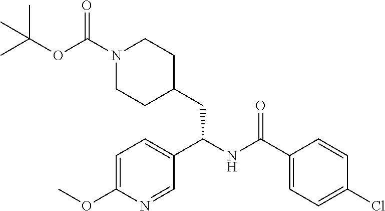 [SMILES: COc1ccc([C@H](CC2CCN(C(=O)OC(C)(C)C)CC2)NC(=O)c2ccc(Cl)cc2)cn1]

The title compound (white solid) was prepared in 31% yield from tert-butyl (S)-4-(2-amino-2-(6-methoxypyridin-3-yl)ethyl)piperidine-1-carboxylate formic acid salt and 4-chlorobenzoic acid as described herein for the preparation of tert-butyl (S)-4-(2-(4-bromobenzamido)-2-(6-methoxypyridin-3-yl)ethyl)piperidine-1-carboxylate. LCMS (ESI) m/z calcd for C₂₅H₃₂ClN₃O₄: 473.2. Found: 474.4 (M+1)⁺. ¹H NMR (400 MHz, CDCl₃) δ 8.19 (d, J=2.3 Hz, 1H), 7.68 (d, J=8.6 Hz, 2H), 7.58 (dd, J=2.3, 8.6 Hz, 1H), 7.40 (d, J=8.6 Hz, 2H), 6.75 (d, J=8.6 Hz, 1H), 6.21 (d, J=7.8 Hz, 1H), 5.25 (q, J=7.8 Hz, 1H), 4.20-3.99 (m, 2H), 3.93 (s, 3H), 2.71-2.51 (m, 2H), 1.96-1.67 (m, 4H), 1.53-1.34 (m, 10H), 1.30-1.08 (m, 2H).

### Example 20: tert-butyl(S)-4-(2-(6-methoxypyridin-3-yl)-2-(5-methylthiophene-2-carboxamido)ethyl)piperidine-1-carboxylate

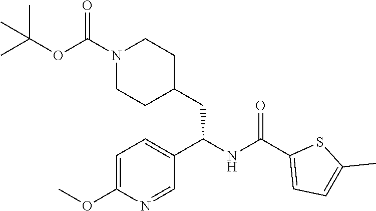 [SMILES: COc1ccc([C@H](CC2CCN(C(=O)OC(C)(C)C)CC2)NC(=O)c2ccc(C)s2)cn1]

A mixture of tert-butyl (S)-4-(2-amino-2-(6-methoxypyridin-3-yl)ethyl)piperidine-1-carboxylate formic acid salt (50 mg, 0.131 mmol) and 5-methylthiophene-2-carboxylic acid (28.0 mg, 0.197 mmol) in DMF (1.3 mL) was treated with DIEA (0.069 mL, 0.393 mmol), and then 50% T3P/EtOAc (0.117 mL, 0.197 mmol) slowly. After stirring for 4 hours at RT, the reaction was diluted with water and extracted with EtOAc. The EtOAc solution was washed with 1N HCl, saturated aqueous NaHCO₃, water, brine, dried over Na₂SO₄, filtered, and concentrated. Purification by reverse phase HPLC (20-90% MeCN/water with 0.1% formic acid) afforded the titled compound (6.7 mg, 11% yield) as white solid. LCMS (ESI) m/z calcd for C₂₄H₃₃N₃O₄S: 459.2. Found: 460.3 (M+1)⁺. ¹H NMR (400 MHz, CDCl₃) δ 8.17 (d, J=2.3 Hz, 1H), 7.57 (dd, J=2.3, 8.6 Hz, 1H), 7.30 (d, J=3.5 Hz, 1H), 6.78-6.69 (m, 2H), 5.93 (d, J=7.8 Hz, 1H), 5.21 (q, J=7.8 Hz, 1H), 4.19-3.97 (m, 2H), 3.93 (s, 3H), 2.74-2.55 (m, 2H), 2.51 (s, 3H), 1.94-1.67 (m, 4H), 1.53-1.36 (m, 10H), 1.30-1.07 (m, 2H).

### Example 21: isopropyl (S)-4-(2-(5-chlorothiophene-2-carboxamido)-2-(6-methoxypyridin-3-yl)ethyl)piperidine-1-carboxylate

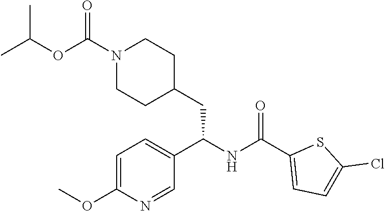 [SMILES: COc1ccc([C@H](CC2CCN(C(=O)OC(C)C)CC2)NC(=O)c2ccc(Cl)s2)cn1]

The title compound was prepared in 86% yield from tert-butyl (S)-4-(2-(5-chlorothiophene-2-carboxamido)-2-(6-methoxypyridin-3-yl)ethyl)piperidine-1-carboxylate and isopropyl chloroformate as described herein for the preparation of phenyl (R)-4-(2-(5-chlorothiophene-2-carboxamido)propyl)piperidine-1-carboxylate. LCMS (ESI) m/z calcd for C₂₂H₂₈ClN₃O₄S: 465.2. Found: 466.3 (M+1)⁺. ¹H NMR (400 MHz, CDCl₃) δ 8.17 (brs, 1H), 7.56 (d, J=7.8 Hz, 1H), 7.24 (br s, 1H), 6.89 (br s, 1H), 6.75 (d, J=8.6 Hz, 1H), 6.03 (d, J=7.0 Hz, 1H), 5.28-5.09 (m, 1H), 4.99-4.76 (m, 1H), 4.32-4.01 (m, 2H), 3.93 (br s, 3H), 2.80-2.50 (m, 2H), 1.93-1.67 (m, 4H), 1.42 (br s, 1H), 1.33-1.03 (m, 8H).

### Example 22: isopropyl (S)-4-(2-(4-chlorobenzamido)-2-(6-methoxypyridin-3-yl)ethyl)piperidine-1-carboxylate

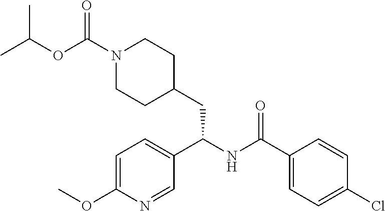 [SMILES: COc1ccc([C@H](CC2CCN(C(=O)OC(C)C)CC2)NC(=O)c2ccc(Cl)cc2)cn1]

The title compound was prepared in 69% yield from tert-butyl (S)-4-(2-(4-chlorobenzamido)-2-(6-methoxypyridin-3-yl)ethyl)piperidine-1-carboxylate and isopropyl chloroformate as described herein for the preparation of phenyl (R)-4-(2-(5-chlorothiophene-2-carboxamido)propyl)piperidine-1-carboxylate. LCMS (ESI) m/z calcd for C₂₄H₃₀ClN₃O₄: 459.2. Found: 460.3 (M+1)⁺. ¹H NMR (400 MHz, CDCl₃) δ 8.19 (s, 1H), 7.68 (d, J=8.2 Hz, 2H), 7.58 (dd, J=2.0, 8.6 Hz, 1H), 7.40 (d, J=8.6 Hz, 2H), 6.75 (d, J=8.6 Hz, 1H), 6.22 (d, J=7.8 Hz, 1H), 5.24 (q, J=7.8 Hz, 1H), 4.97-4.79 (m, 1H), 4.27-4.01 (m, 2H), 3.93 (s, 3H), 2.77-2.54 (m, 2H), 1.96-1.68 (m, 4H), 1.52-1.36 (m, 1H), 1.32-1.09 (m, 8H).

### Synthesis of Amine Intermediate (S)-tert-butyl 4-(2-amino-2-(6-methoxypyridin-3-yl)ethyl)piperidine-1-carboxylate

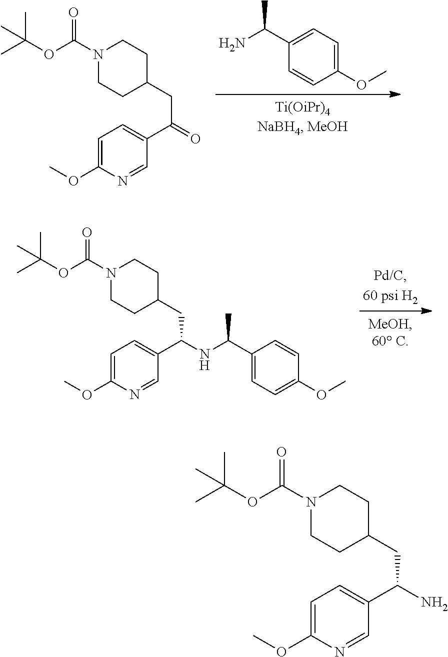

### Step 1: Preparation of tert-butyl 4-((S)-2-(((S)-1-(4-methoxyphenyl)ethyl)amino)-2-(6-methoxypyridin-3-yl)ethyl)piperidine-1-carboxylate

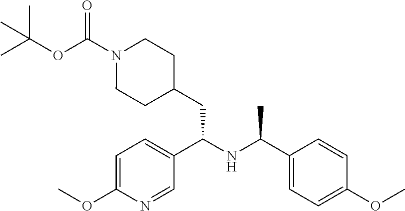 [SMILES: COc1ccc([C@H](C)N[C@@H](CC2CCN(C(=O)OC(C)(C)C)CC2)c2ccc(OC)nc2)cc1]

A suspension of tert-butyl 4-(2-(6-methoxypyridin-3-yl)-2-oxoethyl)piperidine-1-carboxylate (3.00 g, 8.97 mmol) and (S)-1-(4-methoxyphenyl)ethan-1-amine (2.00 mL, 13.5 mmol) in titanium(IV) isopropoxide (7.89 mL, 26.9 mmol) was stirred at 90° C. The reaction progress was monitored by LCMS (aliquots treated with MeOH, NaBH₄, followed by 1N HCl). LCMS indicated complete reaction after 1 hour. The yellow solution was cooled to 0° C., diluted with MeOH (15 mL), treated slowly with NaBH₄ (0.509 g, 13.46 mmol) in portions. After 1 hour, the solution was warmed to RT and stirred for an additional 4 hours. The reaction was quenched with saturated aqueous NH₄Cl and 1N HCl and then extracted with EtOAc. The EtOAc solution was washed with saturated aqueous NaHCO₃, brine, dried over Na₂SO₄, filtered, and concentrated. ¹H-NMR analysis of the crude material showed an approximately 2:1 mixture of SS: RS diastereomers. Purification by flash chromatography (silica gel, 0-100% EtOAc/hexanes, gradient elution) afforded tert-butyl 4-((S)-2-(((S)-1-(4-methoxyphenyl)ethyl)amino)-2-(6-methoxypyridin-3-yl)ethyl)piperidine-1-carboxylate (2.39 g, 57% yield) as clear oil. LCMS (ESI) m/z calcd for C₂₇H₃₉N₃O₄: 469.3. Found: 470.4 (M+1)⁺. ¹H NMR (400 MHz, CDCl₃) δ 7.85 (s, 1H), 7.48 (d, J=8.6 Hz, 1H), 7.12 (d, J=8.2 Hz, 2H), 6.87 (d, J=8.2 Hz, 2H), 6.76 (d, J=8.6 Hz, 1H), 4.08-3.88 (m, 5H), 3.82 (s, 3H), 3.40 (q, J=6.4 Hz, 1H), 3.33 (t, J=6.6 Hz, 1H), 2.67-2.43 (m, 2H), 1.66-1.48 (m, 2H), 1.47-1.16 (m, 15H), 1.09-0.82 (m, 2H).

### Step 2: Preparation of tert-butyl (S)-4-(2-amino-2-(6-methoxypyridin-3-yl)ethyl)piperidine-1-carboxylate

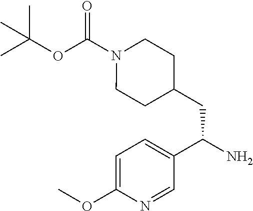 [SMILES: COc1ccc([C@@H](N)CC2CCN(C(=O)OC(C)(C)C)CC2)cn1]

A solution of tert-butyl 4-((S)-2-(((S)-1-(4-methoxyphenyl)ethyl)amino)-2-(6-methoxypyridin-3-yl)ethyl)piperidine-1-carboxylate (2.38 g, 5.07 mmol) in MeOH (51 ml) under N₂ was treated with 10% Pd/C (0.81 g). The mixture was subjected to hydrogenation at 60 psi and 60° C. for 18 hours. After cooling to RT, the mixture was purged with N₂, filtered, washed with MeOH, and concentrated. The crude product was purified by flash chromatography (silica gel, 0-10% MeOH containing 1% NH₄OH/DCM, gradient elution) to give the title compound (1.08 g, 64% yield) as clear oil. Chiral analytical HPLC indicated an enantiomeric purity of 95% [Chiralcel OZ-H column (4.6 mm×250 mm, 5p); mobile phase: 3:7 EtOH/hexane+0.1% DEA; flow rate: 1 mL/min; injection volume: 6 uL (1 mg/mL conc.); monitored at 254 nm]. LCMS (ESI) m/z calcd for C₁₈H₂₉N₃O₃: 335.2. Found: 358.4 (M+23)⁺. ¹H NMR (400 MHz, methanol-d₄) δ 8.12 (d, J=2.0 Hz, 1H), 7.74 (dd, J=2.3, 8.6 Hz, 1H), 6.83 (d, J=8.6 Hz, 1H), 4.12 (t, J=7.6 Hz, 1H), 4.07-3.93 (m, 2H), 3.90 (s, 3H), 2.65 (br. s., 2H), 1.80-1.66 (m, 3H), 1.66-1.54 (m, 1H), 1.43 (s, 9H), 1.38-1.24 (m, 1H), 1.17-1.01 (m, 2H).

### Example 23: tert-butyl (S)-4-(2-(4-bromobenzamido)-2-(6-methoxypyridin-3-yl)ethyl)piperidine-1-carboxylate

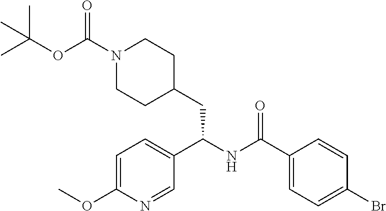 [SMILES: COc1ccc([C@H](CC2CCN(C(=O)OC(C)(C)C)CC2)NC(=O)c2ccc(Br)cc2)cn1]

A solution of tert-butyl (S)-4-(2-amino-2-(6-methoxypyridin-3-yl)ethyl)piperidine-1-carboxylate (50 mg, 0.149 mmol) in DMF (1.5 mL) was treated with 4-bromobenzoic acid (33.0 mg, 0.164 mmol), DIEA (0.078 mL, 0.447 mmol), HATU (85 mg, 0.224 mmol), and stirred at RT for 2 hours. The reaction was quenched with 2M NH₃/MeOH and stirred for an additional 1.5 hours. The mixture was diluted with water and extracted with EtOAc. The EtOAc solution was washed with 1N HCl, saturated aqueous NaHCO₃, brine, dried over Na₂SO₄, filtered, and concentrated. Purification by reverse phase HPLC (C18, 20-90% MeCN/water with 0.1% formic acid) afforded the title compound (60 mg, 77% yield) as white solid. LCMS (ESI) m/z calcd for C₂₅H₃₂BrN₃O₄: 517.2. Found: 518.3 (M+1)⁺. ¹H NMR (400 MHz, CDCl₃) δ 8.19 (d, J=1.6 Hz, 1H), 7.69-7.48 (m, 5H), 6.75 (d, J=8.6 Hz, 1H), 6.22 (d, J=7.8 Hz, 1H), 5.24 (q, J=7.7 Hz, 1H), 4.25-3.98 (m, 2H), 3.93 (s, 3H), 2.74-2.48 (m, 2H), 1.96-1.68 (m, 4H), 1.53-1.34 (m, 10H), 1.31-1.06 (m, 2H).

### Example 24: tert-butyl (S)-4-(2-(5-ethylthiophene-2-carboxamido)-2-(6-methoxypyridin-3-yl)ethyl)piperidine-1-carboxylate

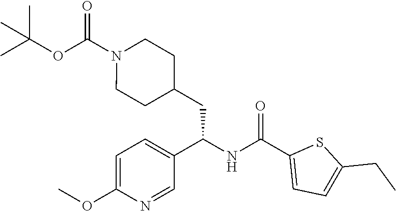 [SMILES: CCc1ccc(C(=O)N[C@@H](CC2CCN(C(=O)OC(C)(C)C)CC2)c2ccc(OC)nc2)s1]

The title compound (white solid) was prepared in 89% yield from tert-butyl (S)-4-(2-amino-2-(6-methoxypyridin-3-yl)ethyl)piperidine-1-carboxylate and 5-ethylthiophene-2-carboxylic acid as described herein for the preparation of tert-butyl (S)-4-(2-(4-bromobenzamido)-2-(6-methoxypyridin-3-yl)ethyl)piperidine-1-carboxylate. LCMS (ESI) m/z calcd for C₂₅H₃₅N₃O₄S: 473.2. Found: 474.3 (M+1)⁺. ¹H NMR (400 MHz, CDCl₃) δ 8.17 (d, J=2.0 Hz, 1H), 7.57 (dd, J=2.1, 8.4 Hz, 1H), 7.33 (d, J=3.5 Hz, 1H), 6.81-6.68 (m, 2H), 5.96 (d, J=8.2 Hz, 1H), 5.22 (q, J=7.8 Hz, 1H), 4.25-3.99 (m, 2H), 3.93 (s, 3H), 2.86 (q, J=7.4 Hz, 2H), 2.73-2.51 (m, 2H), 1.93-1.68 (m, 4H), 1.54-1.38 (m, 10H), 1.32 (t, J=7.6 Hz, 3H), 1.28-1.06 (m, 2H).

### Example 25: tert-butyl (S)-4-(2-(5-fluorothiophene-2-carboxamido)-2-(6-methoxypyridin-3-yl)ethyl)piperidine-1-carboxylate

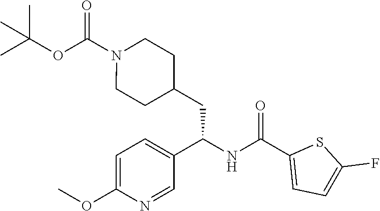 [SMILES: COc1ccc([C@H](CC2CCN(C(=O)OC(C)(C)C)CC2)NC(=O)c2ccc(F)s2)cn1]

The title compound (white solid) was prepared in 32% yield from tert-butyl (S)-4-(2-amino-2-(6-methoxypyridin-3-yl)ethyl)piperidine-1-carboxylate and 5-fluorothiophene-2-carboxylic acid as described herein for the preparation of tert-butyl (S)-4-(2-(6-methoxypyridin-3-yl)-2-(5-methylthiophene-2-carboxamido)ethyl)piperidine-1-carboxylate. LCMS (ESI) m/z calcd for C₂₃H₃₀FN₃O₄S: 463.2. Found: 462.2 (M−1)⁻. ¹H NMR (400 MHz, CDCl₃) δ 8.17 (br s, 1H), 7.56 (d, J=7.8 Hz, 1H), 7.11 (br s, 1H), 6.75 (d, J=8.6 Hz, 1H), 6.47 (d, J=3.5 Hz, 1H), 5.96 (d, J=7.4 Hz, 1H), 5.19 (q, J=7.3 Hz, 1H), 4.26-3.99 (m, 2H), 3.93 (s, 3H), 2.77-2.50 (m, 2H), 1.95-1.66 (m, 4H), 1.53-1.32 (m, 10H), 1.30-1.04 (m, 2H).

### Synthesis of Amine Intermediate tert-butyl 4-(2-amino-4-methylpentyl)piperidine-1-carboxylate

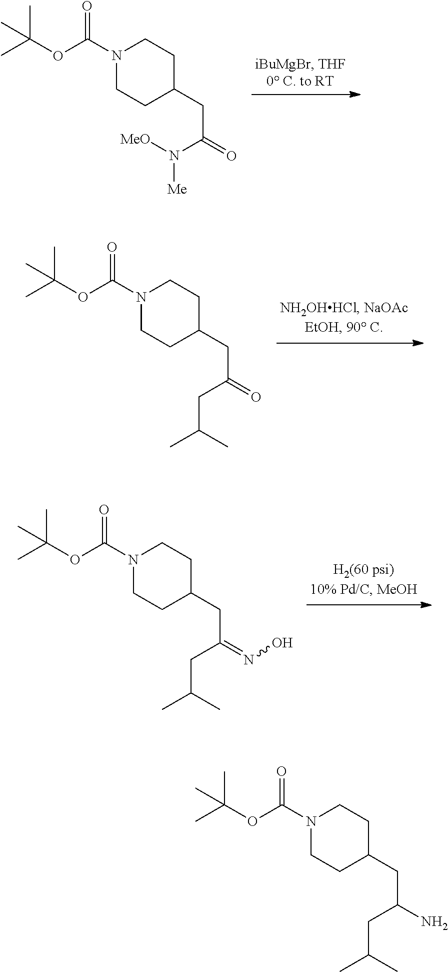 [SMILES: CON(C)C(=O)CC1CCN(C(=O)OC(C)(C)C)CC1 ; CC(C)CC(=O)CC1CCN(C(=O)OC(C)(C)C)CC1 ; CC(C)CC(CC1CCN(C(=O)OC(C)(C)C)CC1)=NO ; CC(C)CC(N)CC1CCN(C(=O)OC(C)(C)C)CC1]

The title compound was prepared in 3 steps from tert-butyl 4-(2-(methoxy(methyl)amino)-2-oxoethyl)piperidine-1-carboxylate and isobutylmagnesium bromide as described herein for the preparation of tert-butyl 4-(2-amino-2-phenylethyl)piperidine-1-carboxylate. LCMS (ESI) m/z calcd for C₁₆H₃₂N₂O₂: 284.3. Found: 307.4 (M+23)⁺. ¹H NMR (400 MHz, CDCl₃) δ 4.27-3.87 (m, 2H), 2.98-2.85 (m, 1H), 2.79-2.61 (m, 2H), 1.80-1.68 (m, 2H), 1.66-1.52 (m, 2H), 1.46 (s, 9H), 1.33-1.19 (m, 4H), 1.18-0.99 (m, 2H), 0.96-0.83 (m, 6H).

### Example 26: tert-butyl 4-(4-methyl-2-(5-methylthiophene-2-carboxamido)pentyl)piperidine-1-carboxylate

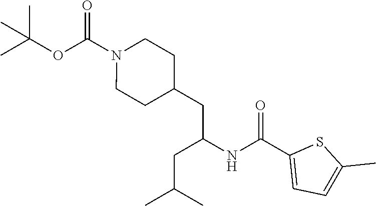 [SMILES: Cc1ccc(C(=O)NC(CC(C)C)CC2CCN(C(=O)OC(C)(C)C)CC2)s1]

The title compound (white solid) was prepared from tert-butyl 4-(2-amino-4-methylpentyl)piperidine-1-carboxylate and 5-methylthiophene-2-carboxylic acid as described herein for the preparation of tert-butyl 4-(2-(5-ethylthiophene-2-carboxamido)-2-phenylethyl)piperidine-1-carboxylate from tert-butyl 4-(2-amino-2-phenylethyl)piperidine-1-carboxylate. LCMS (ESI) m/z calcd for C₂₂H₃₆N₂O₃S: 408.2. Found: 407.5 (M−1)⁻. ¹H NMR (400 MHz, CDCl₃) δ 7.30 (d, J=3.5 Hz, 1H), 6.74 (d, J=3.1 Hz, 1H), 5.43 (d, J=9.4 Hz, 1H), 4.38-4.21 (m, 1H), 4.17-3.91 (m, 2H), 2.77-2.58 (m, 2H), 2.51 (s, 3H), 1.90 (d, J=12.5 Hz, 1H), 1.72-1.54 (m, 3H), 1.53-1.23 (m, 13H), 1.21-1.00 (m, 2H), 1.00-0.84 (m, 6H).

### Example 27: tert-butyl 4-(2-(4-bromobenzamido)-4-methylpentyl)piperidine-1-carboxylate

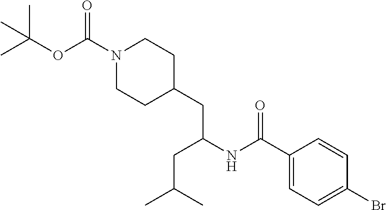 [SMILES: CC(C)CC(CC1CCN(C(=O)OC(C)(C)C)CC1)NC(=O)c1ccc(Br)cc1]

The title compound (white solid) was prepared from tert-butyl 4-(2-amino-4-methylpentyl)piperidine-1-carboxylate and 4-bromobenzoic acid as described herein for the preparation of tert-butyl 4-(2-(5-ethylthiophene-2-carboxamido)-2-phenylethyl)piperidine-1-carboxylate from tert-butyl 4-(2-amino-2-phenylethyl)piperidine-1-carboxylate. LCMS (ESI) m/z calcd for C₂₃H₃₅BrN₂O₃: 466.2. Found: 467.3 (M+1)⁺. ¹H NMR (400 MHz, CDCl₃) δ 7.67-7.54 (m, 4H), 5.69 (d, J=9.4 Hz, 1H), 4.44-4.26 (m, 1H), 4.16-3.92 (m, 2H), 2.76-2.55 (m, 2H), 1.96-1.85 (m, 1H), 1.75-1.55 (m, 3H), 1.54-1.30 (m, 13H), 1.23-1.02 (m, 2H), 1.00-0.88 (m, 6H).

### Example 28: tert-butyl 4-(2-(5-bromothiophene-2-carboxamido)-4-methylpentyl)piperidine-1-carboxylate

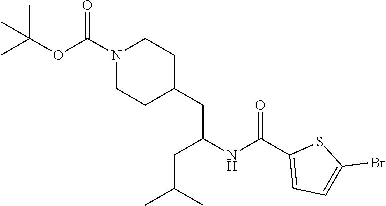 [SMILES: CC(C)CC(CC1CCN(C(=O)OC(C)(C)C)CC1)NC(=O)c1ccc(Br)s1]

The title compound (white solid) was prepared from tert-butyl 4-(2-amino-4-methylpentyl)piperidine-1-carboxylate and 5-bromothiophene-2-carboxylic acid as described herein for the preparation of tert-butyl 4-(2-(5-ethylthiophene-2-carboxamido)-2-phenylethyl)piperidine-1-carboxylate from tert-butyl 4-(2-amino-2-phenylethyl)piperidine-1-carboxylate. LCMS (ESI) m/z calcd for C₂₁H₃₃BrN₂O₃S: 472.1. Found: 471.2 (M−1)⁻. ¹H NMR (400 MHz, methanol-d₄) δ 7.48 (d, J=3.9 Hz, 1H), 7.14 (d, J=3.9 Hz, 1H), 4.31-4.15 (m, 1H), 4.08-3.94 (m, 2H), 2.82-2.55 (m, 2H), 1.97-1.84 (m, 1H), 1.67-1.56 (m, 2H), 1.55-1.23 (m, 14H), 1.19-0.95 (m, 2H), 0.95-0.83 (m, 6H).

### Example 29: tert-butyl (S)-4-(2-(5-bromothiophene-2-carboxamido)-2-(6-methoxypyridin-3-yl)ethyl)piperidine-1-carboxylate

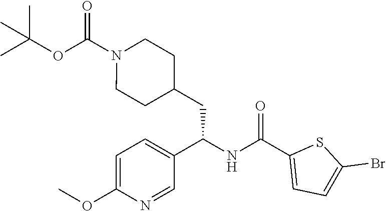 [SMILES: COc1ccc([C@H](CC2CCN(C(=O)OC(C)(C)C)CC2)NC(=O)c2ccc(Br)s2)cn1]

The title compound (white solid) was prepared in 70% yield from tert-butyl (S)-4-(2-amino-2-(6-methoxypyridin-3-yl)ethyl)piperidine-1-carboxylate and 5-bromothiophene-2-carboxylic acid as described herein for the preparation of tert-butyl (S)-4-(2-(4-bromobenzamido)-2-(6-methoxypyridin-3-yl)ethyl)piperidine-1-carboxylate. LCMS (ESI) m/z calcd for C₂₃H₃₀BrN₃O₄S: 523.1. Found: 522.15 (M−1)⁻. ¹H NMR (400 MHz, methanol-d₄) δ 8.12 (d, J=2.3 Hz, 1H), 7.70 (dd, J=2.7, 8.6 Hz, 1H), 7.53 (d, J=3.9 Hz, 1H), 7.15 (d, J=3.9 Hz, 1H), 6.78 (d, J=8.6 Hz, 1H), 5.19-5.09 (m, 1H), 4.09-3.98 (m, 2H), 3.88 (s, 3H), 2.80-2.57 (m, 2H), 1.99-1.87 (m, 1H), 1.83-1.66 (m, 3H), 1.57-1.46 (m, 1H), 1.43 (s, 9H), 1.24-1.06 (m, 2H).

### Example 30: tert-butyl (S)-4-(2-(5-ethylthiophene-2-carboxamido)-2-phenylethyl)piperidine-1-carboxylate

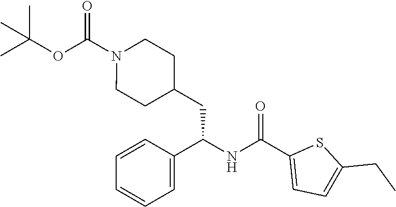 [SMILES: CCc1ccc(C(=O)N[C@@H](CC2CCN(C(=O)OC(C)(C)C)CC2)c2ccccc2)s1]

The title compound (white solid) was prepared in 3 steps from tert-butyl (S,E)-4-(2-((tert-butylsulfinyl)imino)ethyl)piperidine-1-carboxylate and phenylmagnesium bromide as described herein for the preparation of tert-butyl (R)-4-(2-(5-chlorothiophene-2-carboxamido)propyl)piperidine-1-carboxylate. LCMS (ESI) m/z calcd for C₂₅H₃₄N₂O₃S: 442.2. Found: 443.4 (M+1). ¹H NMR (400 MHz, CDCl₃) δ 7.42-7.23 (m, 6H), 6.76 (d, J=3.5 Hz, 1H), 5.99 (d, J=8.2 Hz, 1H), 5.27 (q, J=7.8 Hz, 1H), 4.18-3.93 (m, 2H), 2.85 (q, J=7.7 Hz, 2H), 2.74-2.51 (m, 2H), 1.94-1.67 (m, 4H), 1.45 (s, 10H), 1.35-1.05 (m, 5H).

### Example 31: tert-butyl 4-(2-(5-bromothiophene-2-carboxamido)-2-cyclohexylethyl)piperidine-1-carboxylate

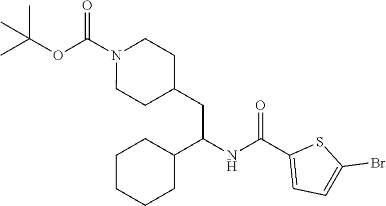 [SMILES: CC(C)(C)OC(=O)N1CCC(CC(NC(=O)c2ccc(Br)s2)C2CCCCC2)CC1]

The title compound (white solid) was prepared in 4 steps from tert-butyl 4-(2-(methoxy(methyl)amino)-2-oxoethyl)piperidine-1-carboxylate and cyclohexylmagnesium chloride as described herein for the preparation of tert-butyl 4-(2-(5-ethylthiophene-2-carboxamido)-2-phenylethyl)piperidine-1-carboxylate LCMS (ESI) m/z calcd for C₂₃H₃₅BrN₂O₃S: 498.2. Found: 499.3 (M+1)⁺. ¹H NMR (400 MHz, methanol-d₄) δ 8.07 (d, J=9.4 Hz, 1H), 7.51 (d, J=3.9 Hz, 1H), 7.14 (d, J=4.3 Hz, 1H), 4.09-3.88 (m, 3H), 2.81-2.52 (m, 2H), 1.92-1.70 (m, 5H), 1.69-1.36 (m, 15H), 1.34-0.89 (m, 7H).

### Example 32: isopropyl (S)-4-(2-(4-chlorobenzamido)-2-phenylethyl)piperidine-1-carboxylate

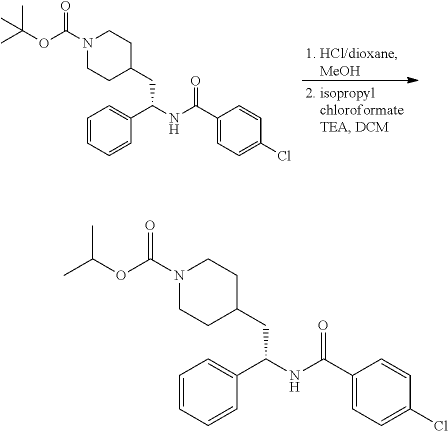 [SMILES: CC(C)(C)OC(=O)N1CCC(C[C@H](NC(=O)c2ccc(Cl)cc2)c2ccccc2)CC1 ; "1. HCl/dioxane,    MeOH" ; CC(C)OC(=O)N1CCC(C[C@H](NC(=O)c2ccc(Cl)cc2)c2ccccc2)CC1]

### Preparation of tert-butyl (S)-4-(2-(4-chlorobenzamido)-2-phenylethyl)piperidine-1-carboxylate

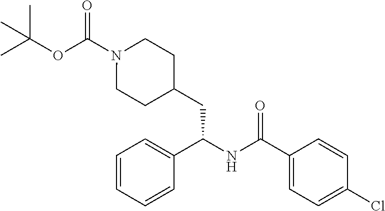 [SMILES: CC(C)(C)OC(=O)N1CCC(C[C@H](NC(=O)c2ccc(Cl)cc2)c2ccccc2)CC1]

The title compound (white solid) was prepared in 3 steps from tert-butyl (S,E)-4-(2-((tert-butylsulfinyl)imino)ethyl)piperidine-1-carboxylate and phenylmagnesium bromide as described herein for the preparation (S)-tert-butyl 4-(2-(5-chlorothiophene-2-carboxamido)-2-cyclopropylethyl)piperidine-1-carboxylate. LCMS (ESI) m/z calcd for C₂₅H₃₁ClN₂O₃: 442.2. Found: 443.4 (M+1)⁺. ¹H NMR (400 MHz, CDCl₃) δ 7.69 (d, J=7.8 Hz, 2H), 7.47-7.29 (m, 7H), 6.21 (d, J=7.8 Hz, 1H), 5.30 (q, J=7.8 Hz, 1H), 4.16-3.96 (m, 2H), 2.75-2.47 (m, 2H), 1.98-1.69 (m, 4H), 1.50-1.33 (m, 10H), 1.24-1.03 (m, 2H).

### Preparation of isopropyl (S)-4-(2-(4-chlorobenzamido)-2-phenylethyl)piperidine-1-carboxylate

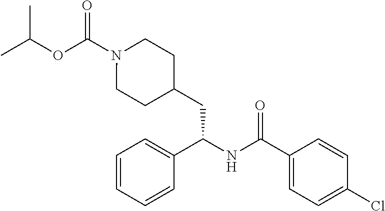 [SMILES: CC(C)OC(=O)N1CCC(C[C@H](NC(=O)c2ccc(Cl)cc2)c2ccccc2)CC1]

The title compound was prepared in 54% yield from tert-butyl (S)-4-(2-(4-chlorobenzamido)-2-phenylethyl)piperidine-1-carboxylate and isopropyl chloroformate as described herein for the preparation of ethyl (S)-4-(2-(5-chlorothiophene-2-carboxamido)-2-cyclopropyl ethyl)piperidine-1-carboxylate. LCMS (ESI) m/z calcd for C₂₄H₂₉ClN₂O₃: 428.2. Found: 429.4 (M+1)⁺. ¹H NMR (400 MHz, methanol-d₄) δ 7.80 (d, J=8.6 Hz, 2H), 7.47 (d, J=8.2 Hz, 2H), 7.41-7.35 (m, 2H), 7.32 (t, J=7.6 Hz, 2H), 7.26-7.19 (m, 1H), 5.23 (dd, J=5.9, 9.8 Hz, 1H), 4.86-4.77 (m, 1H), 4.14-3.93 (m, 2H), 2.89-2.57 (m, 2H), 2.00-1.65 (m, 4H), 1.61-1.46 (m, 1H), 1.30-1.06 (m, 8H).

### Example 33: isopropyl (S)-4-(2-(5-ethylthiophene-2-carboxamido)-2-phenylethyl)piperidine-1-carboxylate

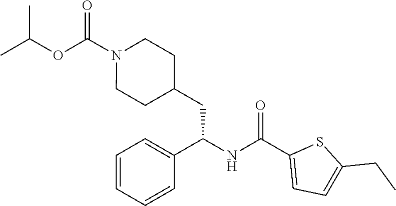 [SMILES: CCc1ccc(C(=O)N[C@@H](CC2CCN(C(=O)OC(C)C)CC2)c2ccccc2)s1]

The title compound (white solid) was prepared in 67% yield from tert-butyl (S)-4-(2-(5-ethylthiophene-2-carboxamido)-2-phenylethyl)piperidine-1-carboxylate and isopropyl chloroformate as described herein for the preparation of ethyl (S)-4-(2-(5-chlorothiophene-2-carboxamido)-2-cyclopropyl ethyl)piperidine-1-carboxylate. LCMS (ESI) m/z calcd for C₂₄H₃₂N₂O₃S: 428.2. Found: 429.3 (M+1)⁺. ¹H NMR (400 MHz, methanol-d₄) δ 7.59 (d, J=3.9 Hz, 1H), 7.41-7.26 (m, 4H), 7.26-7.17 (m, 1H), 6.83 (d, J=3.5 Hz, 1H), 5.18 (dd, J=5.7, 10.0 Hz, 1H), 4.86-4.75 (m, 1H), 4.15-3.99 (m, 2H), 2.85 (q, J=7.5 Hz, 2H), 2.79-2.58 (m, 2H), 2.00-1.64 (m, 4H), 1.63-1.48 (m, 1H), 1.30 (t, J=7.4 Hz, 3H), 1.25-1.04 (m, 8H).

### Example 34: tert-butyl (R)-4-(2-(5-chlorothiophene-2-carboxamido)pent-4-en-1-yl)piperidine-1-carboxylate

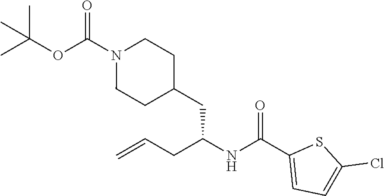 [SMILES: C=CC[C@H](CC1CCN(C(=O)OC(C)(C)C)CC1)NC(=O)c1ccc(Cl)s1]

The title compound (white solid) was prepared in 3 steps from tert-butyl (S,E)-4-(2-((tert-butylsulfinyl)imino)ethyl)piperidine-1-carboxylate and allylmagnesium bromide as described herein for the preparation of (S)-tert-butyl 4-(2-(5-chlorothiophene-2-carboxamido)-2-cyclopropylethyl)piperidine-1-carboxylate. LCMS (ESI) m/z calcd for C₂₀H₂₉ClN₂O₃S: 412.2. Found: 411.2 (M−1)⁻. ¹H NMR (400 MHz, methanol-d₄) δ 7.52 (d, J=3.9 Hz, 1H), 7.01 (d, J=3.9 Hz, 1H), 5.88-5.72 (m, 1H), 5.12-4.96 (m, 2H), 4.23-4.13 (m, 1H), 4.06-3.95 (m, 2H), 2.84-2.53 (m, 2H), 2.37-2.17 (m, 2H), 1.90-1.78 (m, 1H), 1.67-1.57 (m, 1H), 1.57-1.37 (m, 12H), 1.18-0.93 (m, 2H).

### Example 35: phenyl 4-(2-(5-chlorothiophene-2-carboxamido)-4-methylpentyl)piperidine-1-carboxylate

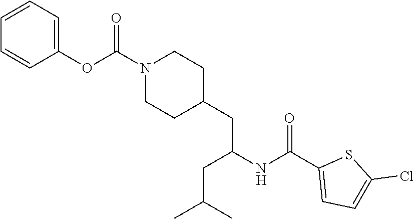

The title compound (white solid) was prepared in 77% yield from tert-butyl 4-(2-(5-chlorothiophene-2-carboxamido)-4-methylpentyl)piperidine-1-carboxylate and phenyl chloroformate as described herein for the preparation of ethyl (S)-4-(2-(5-chlorothiophene-2-carboxamido)-2-cyclopropyl ethyl)piperidine-1-carboxylate. LCMS (ESI) m/z calcd for C₂₃H₂₉ClN₂O₃S: 448.2. Found: 449.3 (M+1)⁺. ¹H NMR (400 MHz, CDCl₃) δ 7.43-7.31 (m, 2H), 7.25 (d, J=3.9 Hz, 1H), 7.23-7.15 (m, 1H), 7.10 (d, J=7.8 Hz, 2H), 6.92 (d, J=3.9 Hz, 1H), 5.44 (d, J=9.0 Hz, 1H), 4.41-4.15 (m, 3H), 3.04-2.65 (m, 2H), 2.11-1.90 (m, 1H), 1.77-1.63 (m, 2H), 1.51-1.13 (m, 7H), 1.02-0.88 (m, 6H).

### Example 36: phenyl (S)-4-(2-(5-chlorothiophene-2-carboxamido)-2-(6-methoxypyridin-3-yl)ethyl)piperidine-1-carboxylate

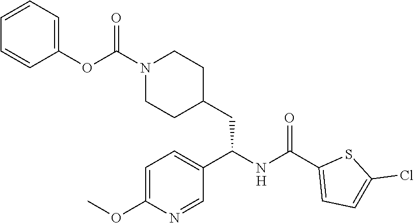

The title compound (off-white solid) was prepared in 74% yield from tert-butyl (S)-4-(2-(5-chlorothiophene-2-carboxamido)-2-(6-methoxypyridin-3-yl)ethyl)piperidine-1-carboxylate and phenyl chloroformate as described herein for the preparation of ethyl (S)-4-(2-(5-chlorothiophene-2-carboxamido)-2-cyclopropyl ethyl)piperidine-1-carboxylate. LCMS (ESI) m/z calcd for C₂₅H₂₆ClN₃O₄S: 499.1. Found: 498.3 (M−1)⁻. ¹H NMR (400 MHz, methanol-d₄) δ 8.15 (d, J=2.3 Hz, 1H), 7.73 (dd, J=2.3, 8.6 Hz, 1H), 7.60 (d, J=3.9 Hz, 1H), 7.42-7.31 (m, 2H), 7.25-7.15 (m, 1H), 7.11-7.00 (m, 3H), 6.80 (d, J=8.6 Hz, 1H), 5.25-5.11 (m, 1H), 4.38-4.06 (m, 2H), 3.89 (s, 3H), 3.06-2.71 (m, 2H), 2.06-1.71 (m, 4H), 1.67-1.53 (m, 1H), 1.45-1.18 (m, 2H).

### Example 37: phenyl (S)-4-(2-(4-chlorobenzamido)-2-(6-methoxypyridin-3-yl)ethyl)piperidine-1-carboxylate

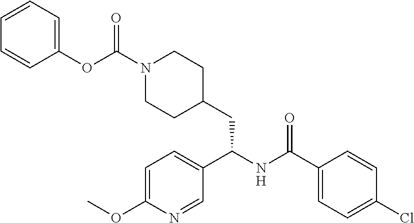

The title compound (off-white solid) was prepared in 68% yield from tert-butyl (S)-4-(2-(4-chlorobenzamido)-2-(6-methoxypyridin-3-yl)ethyl)piperidine-1-carboxylate and phenyl chloroformate as described herein for the preparation of ethyl (S)-4-(2-(5-chlorothiophene-2-carboxamido)-2-cyclopropyl ethyl)piperidine-1-carboxylate. LCMS (ESI) m/z calcd for C₂₇H₂₈ClN₃O₄: 493.2. Found: 494.3 (M+1)⁺. ¹H NMR (400 MHz, methanol-d₄) δ 8.18 (d, J=2.3 Hz, 1H), 7.82 (d, J=8.6 Hz, 2H), 7.76 (dd, J=2.3, 8.6 Hz, 1H), 7.49 (d, J=8.6 Hz, 2H), 7.41-7.32 (m, 2H), 7.26-7.17 (m, 1H), 7.07 (d, J=7.4 Hz, 2H), 6.81 (d, J=8.6 Hz, 1H), 5.32-5.20 (m, 1H), 4.37-4.06 (m, 2H), 3.90 (s, 3H), 3.08-2.76 (m, 2H), 2.07-1.74 (m, 4H), 1.72-1.54 (m, 1H), 1.45-1.19 (m, 2H).

### Example 38: tert-butyl (R)-4-(2-(5-chlorothiophene-2-carboxamido)propyl)piperidine-1-carboxylate

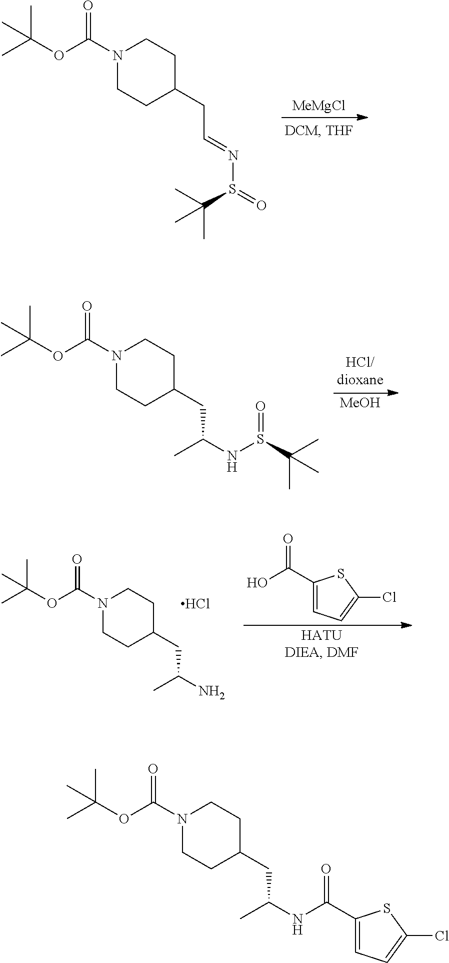

### Step 1: Preparation of tert-butyl 4-((R)-2-(((S)-tert-butylsulfinyl)amino)propyl)piperidine-1-carboxylate

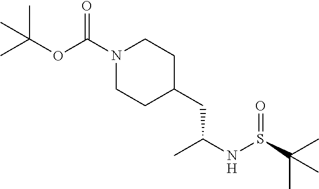

A solution of tert-butyl (S,E)-4-(2-((tert-butylsulfinyl)imino)ethyl)piperidine-1-carboxylate (250 mg, 0.756 mmol) in DCM (19 mL) was treated dropwise with 3M methylmagnesium chloride/THF (0.328 mL, 0.983 mmol), and stirred at RT for 6 hours. The reaction was quenched with saturated aqueous NH₄Cl and extracted with DCM. The DCM solution was washed with water, brine, dried over Na₂SO₄, filtered, and concentrated. Purification by flash chromatography twice (silica gel, 0-10% MeOH/DCM; then 0-100% acetone/hexanes, gradient elution) afforded the title compound (102 mg, 0.293 mmol, 39% yield) as white solid. LCMS (ESI) m/z calcd for C₁₇H₃₄N₂O₃S: 346.2. Found: 347.3 (M+1)⁺. ¹H NMR (400 MHz, CDCl₃) δ 4.23-3.90 (m, 2H), 3.54-3.36 (m, 1H), 2.82 (d, J=8.2 Hz, 1H), 2.74-2.55 (m, 2H), 1.73-1.64 (m, 1H), 1.64-1.54 (m, 3H), 1.51-1.41 (m, 10H), 1.34-1.19 (m, 12H), 1.19-1.00 (m, 2H).

### Step 2: Preparation of tert-butyl (R)-4-(2-aminopropyl)piperidine-1-carboxylate hydrochloride

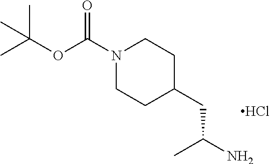 [SMILES: C[C@@H](N)CC1CCN(C(=O)OC(C)(C)C)CC1 ; Cl]

An ice cold solution of tert-butyl 4-((R)-2-(((S)-tert-butylsulfinyl)amino)propyl)piperidine-1-carboxylate (100 mg, 0.289 mmol) in MeOH (1.6 mL) was treated dropwise with 4M HCl/dioxane (0.072 mL, 0.289 mmol). The mixture was stirred in the ice bath for 5 hours, letting the bath to warm up to RT. The reaction was concentrated to dryness and the residue co-evaporated with MeCN, and dried under vacuum to give the title compound as a white solid in quantitative yield. LCMS (ESI) m/z calcd for C₁₃H₂₆N₂O₂: 242.2. Found 243.3 (M+1)⁺. ¹H NMR (400 MHz, methanol-d₄) δ 4.15-3.96 (m, 2H), 3.42-3.33 (m, 1H), 2.88-2.60 (m, 2H), 1.79-1.65 (m, 2H), 1.64-1.42 (m, 12H), 1.29 (d, J=6.6 Hz, 3H), 1.16-1.01 (m, 2H).

### Step 3: Preparation of tert-butyl (R)-4-(2-(5-chlorothiophene-2-carboxamido)propyl)piperidine-1-carboxylate

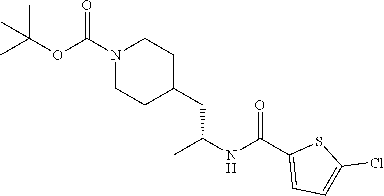

A solution of tert-butyl (R)-4-(2-aminopropyl)piperidine-1-carboxylate hydrochloride (80 mg, 0.29 mmol) in DMF (2.9 mL) was treated with 5-chlorothiophene-2-carboxylic acid (51.3 mg, 0.316 mmol), DIEA (0.200 mL, 1.15 mmol), HATU (164 mg, 0.430 mmol), and stirred at RT for 18 hours. The reaction was quenched with 2M NH₃/MeOH and stirred for an additional 2 hours. The mixture was diluted with water and extracted with EtOAc. The EtOAc solution was washed with 1N HCl, saturated aqueous NaHCO₃, brine, dried over Na₂SO₄, filtered, and concentrated. Purification by flash chromatography (0-80% EtOAc/hexanes) afforded the title compound (90 mg, 77% yield) as white solid. LCMS (ESI) m/z calcd for C₁₈H₂₇ClN₂O₃S: 386.1. Found: 385.4 (M−1)⁻. ¹H NMR (400 MHz, methanol-d₄) δ 7.52 (d, J=3.9 Hz, 1H), 7.00 (d, J=4.3 Hz, 1H), 4.27-4.13 (m, 1H), 4.07-3.96 (m, 2H), 2.84-2.56 (m, 2H), 1.86-1.75 (m, 1H), 1.69-1.31 (m, 13H), 1.19 (d, J=6.2 Hz, 3H), 1.17-0.96 (m, 2H).

### Example 39: (S)-5-chloro-N-(2-(1-(3,3-dimethylbutanoyl)piperidin-4-yl)-1-(6-methoxypyridin-3-yl)ethyl)thiophene-2-carboxamide

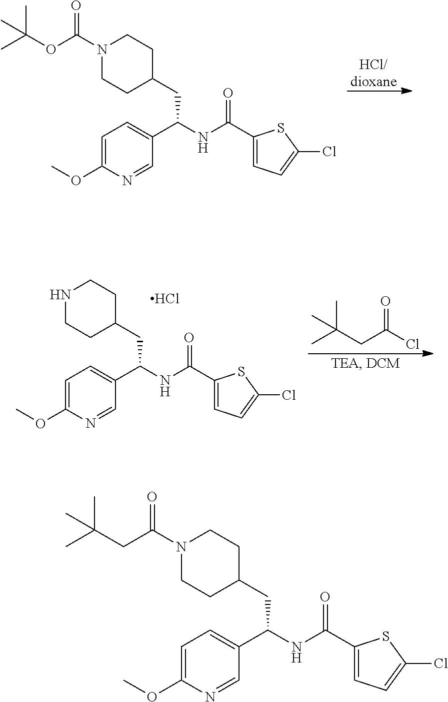

### Step 1: Preparation of (S)-5-chloro-N-(1-(6-methoxypyridin-3-yl)-2-(piperidin-4-yl)ethyl)thiophene-2-carboxamidehydrochloride

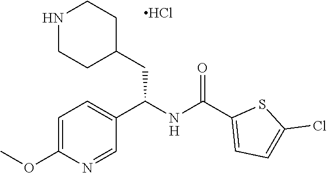

A solution of tert-butyl (S)-4-(2-(5-chlorothiophene-2-carboxamido)-2-(6-methoxypyridin-3-yl)ethyl)piperidine-1-carboxylate (260 mg, 0.542 mmol) in 1,4-dioxane (3.60 mL) and MeOH (1.8 mL) was treated with 4M HCl/dioxane (0.677 mL, 2.71 mmol) and stirred at RT for 18 hours. The mixture was concentrated to dryness at reduced pressure to afford the title compound as a white solid in quantitative yield. LCMS (ESI) m/z calcd for C₁₈H₂₂ClN₃O₂S: 379.1. Found: 380.2 (M+1)⁺.

### Step 2: Preparation of (S)-5-chloro-N-(2-(1-(3,3-dimethylbutanoyl)piperidin-4-yl)-1-(6-methoxypyridin-3-yl)ethyl)thiophene-2-carboxamide

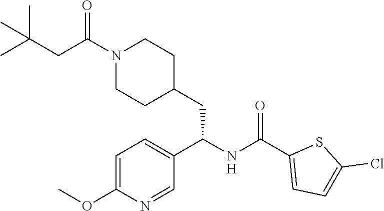

An ice cold solution of (S)-5-chloro-N-(1-(6-methoxypyridin-3-yl)-2-(piperidin-4-yl)ethyl)thiophene-2-carboxamide hydrochloride (55 mg, 0.110 mmol) in DCM (1.1 mL) was treated with TEA (0.046 mL, 0.33 mmol), followed by a solution of 3,3-dimethylbutanoyl chloride (0.018 mL, 0.132 mmol) in DCM (0.5 mL) dropwise. The reaction was warmed to RT for 2.5 hours, diluted with water, and extracted with DCM. The DCM solution was washed with brine, dried over Na₂SO₄, filtered, and concentrated. Purification by reverse phase HPLC (C18, 30-100% MeCN/water with 0.1% formic acid) afforded the title compound (34 mg, 62% yield) as white solid. LCMS (ESI) m/z calcd for C₂₄H₃₂ClN₃O₃S: 477.2. Found: 478.3 (M+1)⁺. ¹H NMR (400 MHz, methanol-d₄) δ 8.13 (s, 1H), 7.71 (d, J=8.6 Hz, 1H), 7.63-7.55 (m, 1H), 7.07-6.96 (m, 1H), 6.79 (d, J=8.6 Hz, 1H), 5.22-5.11 (m, 1H), 4.63-4.48 (m, 1H), 4.12-3.98 (m, 1H), 3.88 (s, 3H), 3.10-2.95 (m, 1H), 2.54 (q, J=12.8 Hz, 1H), 2.42-2.18 (m, 2H), 2.04-1.50 (m, 5H), 1.33-1.07 (m, 2H), 1.02 (s, 9H).

### Example 40: (S)-N-(tert-butyl)-4-(2-(5-chlorothiophene-2-carboxamido)-2-(6-methoxypyridin-3-yl)ethyl)piperidine-1-carboxamide

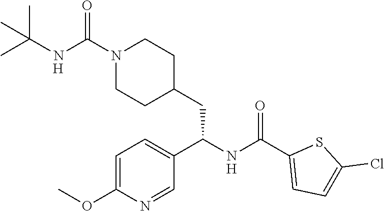

A solution of (S)-5-chloro-N-(1-(6-methoxypyridin-3-yl)-2-(piperidin-4-yl)ethyl)thiophene-2-carboxamide hydrochloride (55 mg, 0.110 mmol) in DCM (1.1 mL) was treated with TEA (0.061 mL, 0.44 mmol), followed by a solution of t-butyl isocyanate (0.025 mL, 0.22 mmol) in DCM (0.5 mL) dropwise. The reaction was stirred at RT for 3 hours, diluted with water and 1N HCl and extracted with DCM. The DCM solution was washed with saturated aqueous NaHCO₃, brine, dried over Na₂SO₄, filtered, and concentrated. Purification by reverse phase HPLC (C18, 30-100% MeCN/water with 0.1% formic acid) afforded the title compound (28 mg, 50% yield) as white solid. LCMS (ESI) m/z calcd for C₂₃H₃₁ClN₄O₃S: 478.2. Found: 479.4 (M+1)⁺. ¹H NMR (400 MHz, methanol-d₄) δ 8.12 (d, J=2.3 Hz, 1H), 7.70 (dd, J=2.7, 8.6 Hz, 1H), 7.58 (d, J=3.9 Hz, 1H), 7.02 (d, J=3.9 Hz, 1H), 6.78 (d, J=8.6 Hz, 1H), 5.58 (s, 1H), 5.19-5.10 (m, 1H), 4.00-3.90 (m, 2H), 3.88 (s, 3H), 2.74-2.56 (m, 2H), 2.01-1.86 (m, 1H), 1.83-1.64 (m, 3H), 1.57-1.42 (m, 1H), 1.30 (s, 9H), 1.26-1.07 (m, 2H).

### Example 41: (S)-5-chloro-N-(2-(1-(isobutylsulfonyl)piperidin-4-yl)-1-(6-methoxypyridin-3-yl)ethyl)thiophene-2-carboxamide

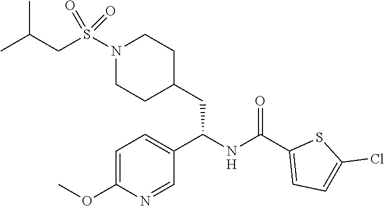

An ice cold solution of (S)-5-chloro-N-(1-(6-methoxypyridin-3-yl)-2-(piperidin-4-yl)ethyl)thiophene-2-carboxamide hydrochloride (55 mg, 0.110 mmol) in DCM (1.1 mL) was treated with TEA (0.046 mL, 0.33 mmol), followed by a solution of isobutanesulfonyl chloride (0.029 mL, 0.22 mmol) in DCM (0.5 mL) dropwise. The reaction was warmed to RT for 3.5 hours, treated with additional isobutanesulfonyl chloride (25 uL), stirred at 40° C. for 1 hour, and then cooled to RT overnight. The mixture was diluted with water and extracted with DCM The DCM solution was washed with brine, dried over Na₂SO₄, filtered, and concentrated. Purification by reverse phase HPLC (C18, 30-100% MeCN/water with 0.1% formic acid) afforded the title compound (28 mg, 50% yield) as a white solid. LCMS (ESI) m/z calcd for C₂₂H₃₀ClN₃O₄S₂: 499.1. Found: 500.3 (M+1)⁺. ¹H NMR (400 MHz, CDCl₃) δ 8.17 (d, J=1.6 Hz, 1H), 7.56 (dd, J=2.0, 8.6 Hz, 1H), 7.23 (d, J=3.9 Hz, 1H), 6.89 (d, J=3.5 Hz, 1H), 6.75 (d, J=8.6 Hz, 1H), 5.96 (d, J=8.2 Hz, 1H), 5.20 (q, J=7.7 Hz, 1H), 3.93 (s, 3H), 3.84-3.67 (m, 2H), 2.77-2.54 (m, 4H), 2.34-2.18 (m, 1H), 2.00-1.74 (m, 4H), 1.49-1.29 (m, 3H), 1.09 (d, J=6.6 Hz, 6H).

### Example 42: phenyl (R)-4-(2-(5-chlorothiophene-2-carboxamido)propyl)piperidine-1-carboxylate

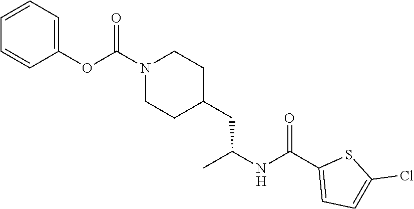

A solution of tert-butyl (R)-4-(2-(5-chlorothiophene-2-carboxamido)propyl)piperidine-1-carboxylate (56 mg, 0.145 mmol) in 1,4-dioxane (1 mL) and MeOH (0.5 mL) was treated with 4M HCl/dioxane (0.181 mL, 0.724 mmol), stirred at RT for 5 hours and then concentrated to dryness at reduced pressure. The residue was suspended in DCM (1 mL). The mixture was treated with TEA (0.061 mL, 0.43 mmol), followed by phenyl chloroformate (0.027 mL, 0.22 mmol). After stirring at RT for 30 minutes, the mixture was diluted with water and extracted with DCM. The DCM solution was washed with brine, dried over Na₂SO₄, filtered, and concentrated. Purification by reverse phase HPLC (C18, 30-100% MeCN/water with 0.1% formic acid) afforded the title compound (44 mg, 72% yield) as white solid. LCMS (ESI) m/z calcd for C₂₀H₂₃ClN₂O₃S: 406.1. Found: 407.3 (M+1)⁺. ¹H NMR (400 MHz, methanol-d₄) δ 8.24 (d, J=8.6 Hz, 1H), 7.54 (d, J=3.9 Hz, 1H), 7.41-7.30 (m, 2H), 7.25-7.15 (m, 1H), 7.06 (d, J=7.8 Hz, 2H), 7.01 (d, J=4.3 Hz, 1H), 4.36-4.06 (m, 3H), 3.08-2.74 (m, 2H), 1.98-1.85 (m, 1H), 1.81-1.69 (m, 1H), 1.67-1.52 (m, 2H), 1.49-1.39 (m, 1H), 1.36-1.06 (m, 5H).

### Example 43: (S)-5-chloro-N-(2-(1-(2,2-difluoro-2-phenylacetyl)piperidin-4-yl)-1-(6-methoxypyridin-3-yl)ethyl)thiophene-2-carboxamide

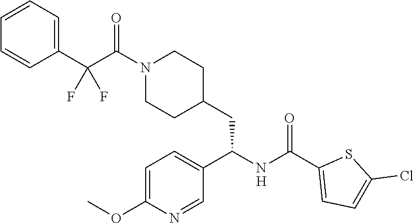

A solution of (S)-5-chloro-N-(1-(6-methoxypyridin-3-yl)-2-(piperidin-4-yl)ethyl)thiophene-2-carboxamide hydrochloride (40 mg, 0.080 mmol) in DMF (0.8 mL) was treated with 2,2-difluoro-2-phenylacetic acid (15 mg, 0.088 mmol), DIEA (0.042 mL, 0.24 mmol), HATU (46 mg, 0.120 mmol), and stirred at RT for 3.5 hours. The reaction was quenched with 2M NH₃/MeOH and stirred for an additional 45 min. The mixture was diluted with water and extracted with EtOAc. The EtOAc solution was washed with 1N HCl, saturated aqueous NaHCO₃, brine, dried over Na₂SO₄, filtered, and concentrated. Purification by reverse phase HPLC (C18, 30-100% MeCN/water with 0.1% formic acid) afforded the title compound (24 mg, 55% yield) as white solid. LCMS (ESI) m/z calcd for C₂₆H₂₆ClF₂N₃O₃S: 533.1. Found: 534.3 (M+1)⁺. ¹H NMR (400 MHz, CDCl₃) δ 8.14 (brs, 1H), 7.64-7.38 (m, 6H), 7.22 (d, J=3.5 Hz, 1H), 6.88 (d, J=3.9 Hz, 1H), 6.75 (d, J=8.6 Hz, 1H), 5.96 (br s, 1H), 5.26-5.04 (m, 1H), 4.73-4.46 (m, 1H), 4.06-3.79 (m, 4H), 2.91-2.50 (m, 2H), 1.96-1.67 (m, 4H), 1.56-1.38 (m, 1H), 1.34-1.09 (m, 1H), 1.07-0.76 (m, 1H).

### Example 44: tert-butyl 4-(2-(5-chlorothiophene-2-carboxamido)-2-(5-(diethylamino)-1,3,4-oxadiazol-2-yl)ethyl)piperidine-1-carboxylate

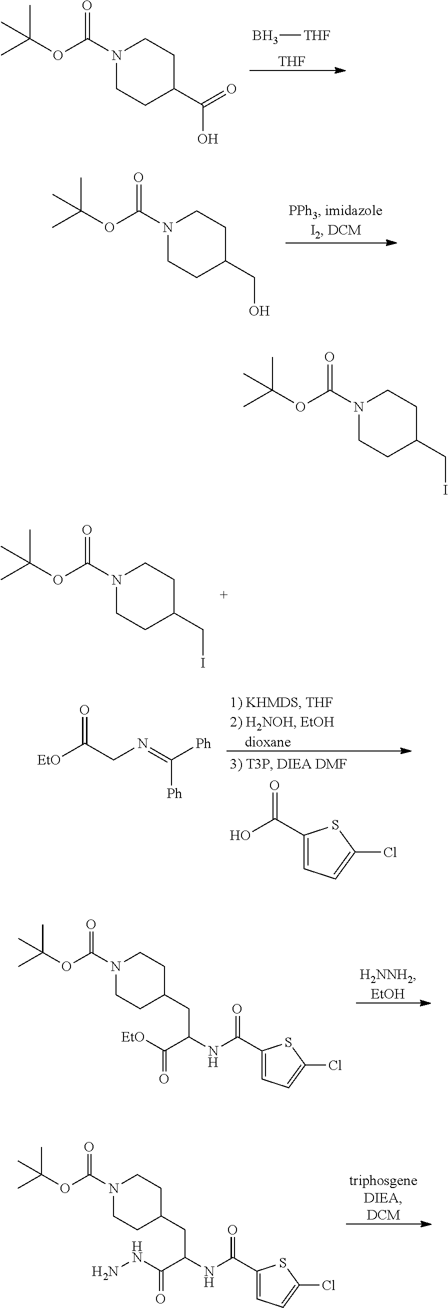

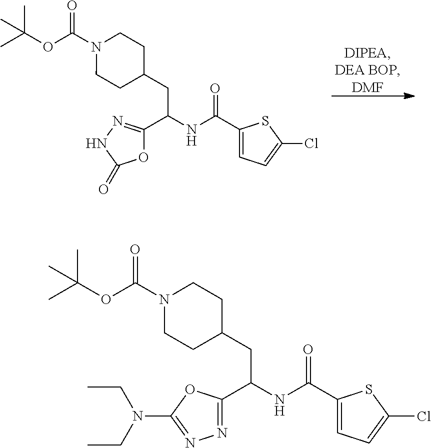 [SMILES: CC(C)(C)OC(=O)N1CCC(CC(NC(=O)c2ccc(Cl)s2)c2n[nH]c(=O)o2)CC1 ; "DIPEA,DEA BOP,DMF" ; CCN(CC)c1nnc(C(CC2CCN(C(=O)OC(C)(C)C)CC2)NC(=O)c2ccc(Cl)s2)o1]

### Step 1: Preparation of tert-butyl 4-(hydroxymethyl)piperidine-1-carboxylate

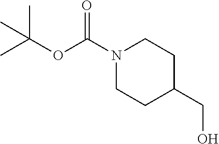

To an ice cold solution of 1-(tert-butoxycarbonyl)piperidine-4-carboxylic acid (5.00 g, 21.8 mmol) in THF (50 mL) was slowly added 1M BH₃-THF in THF (32.7 mL, 32.7 mmol) and the mixture was allowed to stir at 0° C. for 2 hours after which time TLC (10% MeOH/DCM, KMnO₄ stain) indicated complete reaction. MeOH (5 mL) was added dropwise and the mixture was stirred at ambient temperature for 10 minutes. Saturated NaHCO₃ (50 mL) was added and the mixture was extracted with EtOAc. The extracts were washed with brine, dried over Na₂SO₄, filtered and concentrated to afford the title compound as a viscous colorless oil (3.53 g, 75% yield). ¹H NMR (400 MHz, DMSO-d₆) δ 4.45 (t, J=5.3 Hz, 1H), 4.00-3.85 (m, 2H), 3.23 (t, J=5.8 Hz, 2H), 2.79-2.53 (m, 2H), 1.66-1.56 (m, 2H), 1.55-1.44 (m, 1H), 1.43-1.33 (m, 9H), 1.01-0.90 (m, 2H).

### Step 2: Preparation of tert-butyl 4-(iodomethyl)piperidine-1-carboxylate

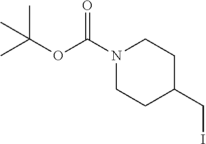

To a stirred solution of tert-butyl 4-(hydroxymethyl)piperidine-1-carboxylate (3.53 g, 16.4 mmol), triphenylphosphine (6.86 g, 26.2 mmol) and imidazole (1.78 g, 26.2 mmol) in DCM (100 mL) at 0° C. was added iodine (6.64 g, 26.2 mmol). The mixture was stirred at 0° C. for 5 minutes, then warmed to ambient temperature and stirred overnight (excluded from light by wrapping vessel in aluminum foil after removing from ice bath). The yellow-brown reaction mixture was diluted with hexanes (200 mL) and the triphenylphosphine-oxide precipitate was filtered off. Hexanes (200 mL) was added to the filtrate (some additional precipitate and a reddish-brown oily residue was observed) and the mixture was filtered once more to remove the solids. The filtrate was concentrated and the residue was purified by flash chromatography (silica gel, 0-40% EtOAc/hexanes, gradient elution) to afford the title compound as a colorless oil (3.96 g, 74% yield). ¹H NMR (400 MHz, CDCl₃) δ 4.34-3.95 (m, 2H), 3.10 (d, J=6.4 Hz, 2H), 2.69 (t, J=12.0 Hz, 2H), 1.83 (d, J=13.2 Hz, 2H), 1.69-1.54 (m, 1H), 1.46 (s, 9H), 1.14 (dq, J=4.2, 12.3 Hz, 2H).

### Step 3: Preparation of tert-butyl 4-(2-(5-chlorothiophene-2-carboxamido)-3-ethoxy-3-oxopropyl)piperidine-1-carboxylate

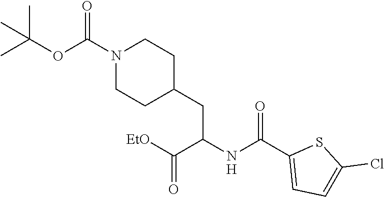

To a solution of ethyl 2-((diphenylmethylene)amino)acetate (2.60 g, 9.73 mmol) in THE (60 mL) at −78° C. was added 1M potassium bis(trimethylsilyl)amide/THF (12.16 mL, 12.16 mmol) and the resulting yellow solution was stirred at −78° C. for 30 minutes. A solution of tert-butyl 4-(iodomethyl)piperidine-1-carboxylate (3.95 g, 12.16 mmol) in THE (15 mL) was slowly added. The reaction mixture was stirred at −78° C. for 30 minutes, 0° C. for one hour and then warmed to ambient temperature and stirred overnight. A solution of citric acid (2.34 g, 12.2 mmol) in water (100 mL) was added and the mixture was diluted with EtOAc. The mixture was partitioned and separated. The aqueous phase was further extracted with EtOAc and the combined organic phases were dried over MgSO₄, filtered and concentrated. The residue was purified by flash chromatography (silica gel, 0-50% EtOAc/hexanes, gradient elution) to afford a pale yellow residue (3.6 g). The purified residue was dissolved in ethanol (80 mL), treated with 50 wt % aqueous hydroxylamine (2.50 mL, 40.8 mmol), stirred for 5 minutes and then treated with acetic acid (2.50 mL, 43.7 mmol). The reaction mixture was stirred overnight at ambient temperature. Brine (150 mL) was added and the mixture was made slightly basic by adding 1.0 N NaOH. The mixture was extracted once with EtOAc and twice with DCM. The combined extracts were dried over Na₂SO₄, filtered and concentrated to a pale yellow residue. To a solution of the crude residue, 5-chlorothiophene-2-carboxylic acid (1.26 g, 7.75 mmol) and DIEA (2.03 mL, 11.6 mmol) in DMF (25 mL) was added 50% T3P/EtOAc (7.38 mL, 12.4 mmol) and the mixture was stirred at ambient temperature for approximately two hours. The mixture was partitioned between EtOAc and saturated aqueous NaHCO₃. The layers were separated and the aqueous phase was further extracted with EtOAc. The combined organic extracts were washed with water, then brine, dried over Na₂SO₄, filtered and concentrated. The residue was purified by flash chromatography (silica gel, 0-40% EtOAc/hexanes, gradient elution) to afford the title compound a white foam (1.60 g, 37% yield). LCMS (ESI) m/z calcd for C₂₀H₂₉ClN₂O₅S: 444.2. Found: 445.3 (M+1)⁺. ¹H NMR (400 MHz, CDCl₃) δ 7.32 (d, J=3.8 Hz, 1H), 6.92 (d, J=3.8 Hz, 1H), 6.43 (d, J=8.2 Hz, 1H), 4.80 (dt, J=5.3, 8.4 Hz, 1H), 4.23 (q, J=7.1 Hz, 2H), 4.17-3.98 (m, 2H), 2.77-2.57 (m, 2H), 1.90-1.76 (m, 2H), 1.73-1.61 (m, 2H), 1.60-1.50 (m, 1H), 1.45 (s, 9H), 1.31 (t, J=7.1 Hz, 3H), 1.23-1.06 (m, 2H).

### Step 4: Preparation of tert-butyl 4-(2-(5-chlorothiophene-2-carboxamido)-3-hydrazinyl-3-oxopropyl)piperidine-1-carboxylate

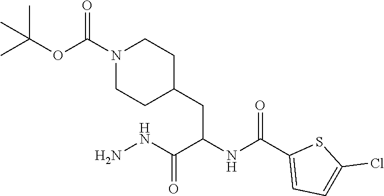 [SMILES: CC(C)(C)OC(=O)N1CCC(CC(NC(=O)c2ccc(Cl)s2)C(=O)NN)CC1]

A solution of tert-butyl 4-(2-(5-chlorothiophene-2-carboxamido)-3-ethoxy-3-oxopropyl)piperidine-1-carboxylate (500 mg, 1.12 mmol) in ethanol (8.0 mL) was treated with hydrazine (0.176 mL, 5.62 mmol) and then stirred overnight at ambient temperature. LCMS indicated ˜50% conversion to the desired product. Additional hydrazine (0.176 mL, 5.62 mmol) was added and the mixture was stirred at ambient temperature for seven hours. LCMS indicated 90% completion. Additional hydrazine (0.176 mL, 5.62 mmol) was added and then stirred for three days. The mixture was concentrated and then placed under vacuum to afford the title compound as an off-white solid in quantitative yield. LCMS (ESI) m/z calcd for C₁₈H₂₇ClN₄O₄S: 430.1. Found: 431.3 (M+1)⁺.

### Step 5: Preparation of tert-butyl 4-(2-(5-chlorothiophene-2-carboxamido)-2-(5-oxo-4,5-dihydro-1,3,4-oxadiazol-2-yl)ethyl)piperidine-1-carboxylate

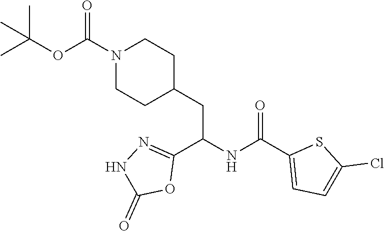 [SMILES: CC(C)(C)OC(=O)N1CCC(CC(NC(=O)c2ccc(Cl)s2)c2n[nH]c(=O)o2)CC1]

A suspension of tert-butyl 4-(2-(5-chlorothiophene-2-carboxamido)-3-hydrazinyl-3-oxopropyl)piperidine-1-carboxylate (521 mg, 1.21 mmol) and DIEA (0.422 mL, 2.418 mmol) in DCM (5.0 mL) was treated with a solution of triphosgene (143 mg, 0.484 mmol) in DCM (1.0 mL, sonicated until triphosgene dissolved) to give a yellow solution. An exotherm was observed and the mixture was stirred at ambient temperature for 30 minutes. The mixture was concentrated and then purified by flash chromatography (silica gel, 0-10% MeOH/DCM, gradient elution) to afford the title compound as a colorless residue (351 mg, 37% yield). LCMS (ESI) m/z calcd for C₁H₂₅ClN₄O₅S: 456.1. Found: 457.2 (M+1)⁺.

### Step 6: Preparation of tert-butyl 4-(2-(5-chlorothiophene-2-carboxamido)-2-(5-(diethylamino)-1,3,4-oxadiazol-2-yl)ethyl)piperidine-1-carboxylate

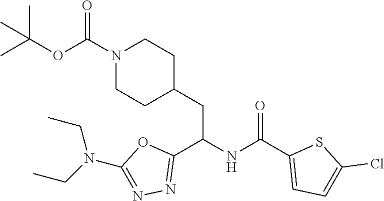 [SMILES: CCN(CC)c1nnc(C(CC2CCN(C(=O)OC(C)(C)C)CC2)NC(=O)c2ccc(Cl)s2)o1]

To a solution of tert-butyl 4-(2-(5-chlorothiophene-2-carboxamido)-2-(5-oxo-4,5-dihydro-1,3,4-oxadiazol-2-yl)ethyl)piperidine-1-carboxylate (50 mg, 0.109 mmol) in DMF (1.09 mL) was sequentially added DIEA (38.2 μl, 0.219 mmol) and diethylamine (22.9 μl, 0.219 mmol). After stirring for several minutes, BOP (53.2 mg, 0.120 mmol) was added and the mixture was stirred at ambient temperature overnight. The mixture was purified directly by reverse phase HPLC (C18, 10-100% MeCN/water with 0.1% formic acid) to afford the title compound as a white solid (28 mg, 50% yield). LCMS (ESI) m/z calcd for C₂₃H₃₄ClN₅O₄S: 511.2. Found: 512.4 (M+1)⁺. ¹H NMR (400 MHz, CDCl₃) δ 7.40 (d, J=3.9 Hz, 1H), 6.79 (d, J=3.9 Hz, 1H), 5.50-5.37 (m, 1H), 4.21-3.90 (m, 2H), 3.42 (q, J=7.0 Hz, 4H), 2.66 (m, 2H), 1.90-1.58 (m, 5H), 1.43 (s, 9H), 1.20 (t, J=7.2 Hz, 8H).

### Example 45: ethyl 4-(2-(5-chlorothiophene-2-carboxamido)-2-(5-(diethylamino)-1,3,4-oxadiazol-2-yl)ethyl)piperidine-1-carboxylate

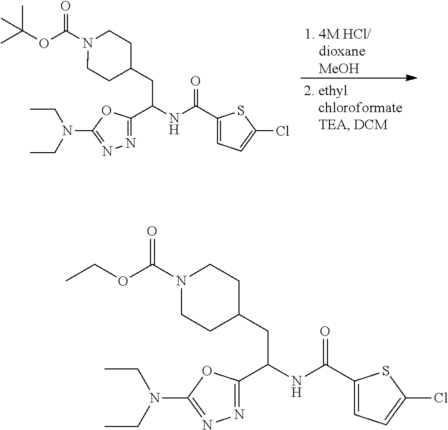 [SMILES: CCN(CC)c1nnc(C(CC2CCN(C(=O)OC(C)(C)C)CC2)NC(=O)c2ccc(Cl)s2)o1 ; "1. 4M HCl/    dioxane    MeOH" ; CCOC(=O)N1CCC(CC(NC(=O)c2ccc(Cl)s2)c2nnc(N(CC)CC)o2)CC1]

A solution of tert-butyl 4-(2-(5-chlorothiophene-2-carboxamido)-2-(5-(diethylamino)-1,3,4-oxadiazol-2-yl)ethyl)piperidine-1-carboxylate (108 mg, 0.211 mmol) in methanol (1.0 mL) was treated with 4M HCl/dioxane (2.0 mL, 8.00 mmol). The mixture was stirred at ambient temperature for 30 minutes and then concentrated to a pale yellow residue. The residue was suspended in TEA (0.118 mL, 0.844 mmol) and DCM (2.0 mL) and then treated with a solution of ethyl chloroformate (0.024 mL, 0.253 mmol) in DCM (76 uL). The mixture was allowed to stir at ambient temperature for 45 minutes. The mixture was partitioned between DCM and saturated aqueous NaHCO₃ and the phases were separated. The aqueous phase was extracted with DCM and the combined organic phases were dried over MgSO₄, filtered and concentrated. The residue was purified by flash chromatography (silica gel, 0-100% EtOAc/hexanes, gradient elution) to afford the title compound as a white solid (73 mg, 72% yield). LCMS (ESI) m/z calcd for C₂₁H₃₀ClN₅O₄S: 483.2. Found: 484.4 (M+1)⁺. ¹H NMR (400 MHz, CD₃OD) δ 8.92 (d, J=8.6 Hz, 1H), 7.59 (d, J=3.9 Hz, 1H), 7.04 (d, J=4.3 Hz, 1H), 5.38-5.32 (m, 1H), 4.16-4.05 (m, 4H), 3.44 (q, J=7.0 Hz, 4H), 2.89-2.65 (m, 2H), 1.99-1.93 (m, 2H), 1.88-1.71 (m, 2H), 1.70-1.57 (m, 1H), 1.28-1.07 (m, 11H).

### Example 46: tert-butyl 4-(3-methyl-2-(5-methylthiophene-2-carboxamido)butyl)piperidine-1-carboxylate

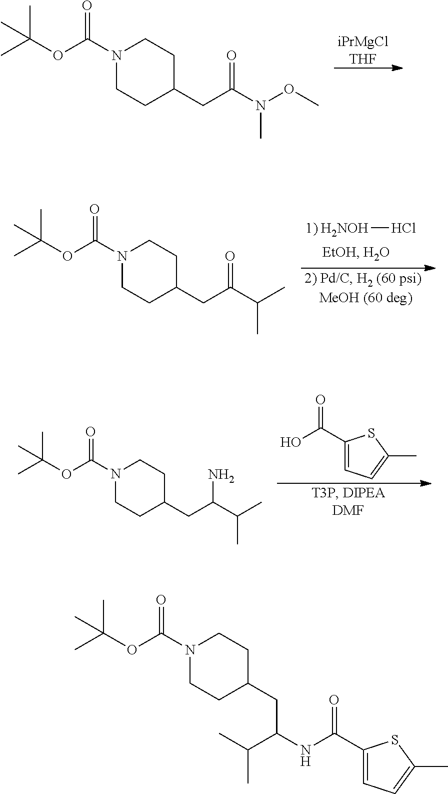

### Step 1: Preparation of tert-butyl 4-(3-methyl-2-oxobutyl)piperidine-1-carboxylate

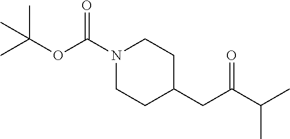 [SMILES: CC(C)C(=O)CC1CCN(C(=O)OC(C)(C)C)CC1]

To a solution of tert-butyl 4-(2-(methoxy(methyl)amino)-2-oxoethyl)piperidine-1-carboxylate (1.04 g, 3.61 mmol) in THE (20 mL) at 0° C. was added 2M iPrMgCl/THF by dropwise addition. After stirring at 0° C. for 5 minutes, the solution was allowed to warm to RT. After 80 minutes, the mixture was cooled to 0° C., then treated slowly with additional 2M iPrMgCl/THF (4.52 mL, 9.04 mmol) and stirred for several minutes at ice bath temperature. The ice bath was removed and the mixture was allowed to stir at ambient temperature overnight. Saturated NH₄Cl was added, the mixture was stirred for 10 minutes and then extracted with EtOAc. The extracts were washed with saturated NaHCO₃, then brine, dried over Na₂SO₄, filtered and concentrated. The residue was purified by flash chromatography (silica gel, 0-70% EtOAc/hexanes, gradient elution) to afford the title compound as a colorless residue (0.294 g, 30% yield). ¹H NMR (400 MHz, CDCl₃) δ 4.16-3.91 (m, 1H), 2.82-2.62 (m, 2H), 2.60-2.49 (m, 1H), 2.40-2.32 (m, 2H), 2.07-1.93 (m, 1H), 1.67-1.57 (m, 3H), 1.47-1.40 (m, 9H), 1.16-0.98 (m, 8H).

### Step 2: Preparation of tert-butyl 4-(2-amino-3-methylbutyl)piperidine-1-carboxylate

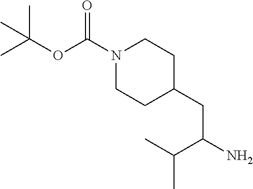 [SMILES: CC(C)C(N)CC1CCN(C(=O)OC(C)(C)C)CC1]

A solution of tert-butyl 4-(3-methyl-2-oxobutyl)piperidine-1-carboxylate (294 mg, 1.091 mmol), sodium acetate (448 mg, 5.46 mmol) and hydroxylamine hydrochloride (152 mg, 2.18 mmol) in ethanol (6.0 mL) and water (3.0 mL) was stirred at 90° C. for 150 minutes. The reaction was cooled to ambient temperature, water was added and then extracted with EtOAc. The extracts were washed with brine, dried over Na₂SO₄, filtered and concentrated to give the crude oxime product as a viscous colorless oil. A solution of the crude oxime product in methanol (8 mL) was purged with nitrogen, treated with 10% Pd/C (40 mg, 0.376 mmol) and then stirred under hydrogen (60 psi) at 60° C. for three days. TLC indicated starting material still remained. The mixture was purged with nitrogen, additional 10% Pd/C (40 mg, 0.376 mmol) added and then stirred under hydrogen (60 psi) at 60° C. overnight. The mixture was cooled to ambient temperature, filtered through a PTFE filter and then concentrated. The residue was purified by flash chromatography (silica gel, 0-10% MeOH/DCM, MeOH containing 1% NH₄OH, gradient elution) to afford the title compounds as a colorless residue (201 mg, 68%). ¹H NMR (400 MHz, CDCl₃) δ 4.22-3.94 (m, 1H), 2.78-2.56 (m, 3H), 1.77-1.68 (m, 1H), 1.64-1.49 (m, 3H), 1.44 (s, 9H), 1.30-0.94 (m, 5H), 0.90-0.81 (m, 6H).

### Step 3: Preparation of tert-butyl 4-(3-methyl-2-(5-methylthiophene-2-carboxamido)butyl)piperidine-1-carboxylate

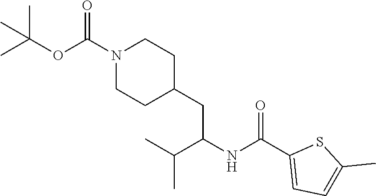 [SMILES: Cc1ccc(C(=O)NC(CC2CCN(C(=O)OC(C)(C)C)CC2)C(C)C)s1]

A solution of tert-butyl 4-(2-amino-3-methylbutyl)piperidine-1-carboxylate (41 mg, 0.152 mmol), 5-methylthiophene-2-carboxylic acid (32.3 mg, 0.227 mmol) and DIEA (0.048 mL, 0.273 mmol) in DMF (1.0 mL) was treated with 50% T3P/EtOAc (0.144 mL, 0.243 mmol) and the mixture was allowed to stir at ambient temperature for 140 minutes. Additional 5-chlorothiophene-2-carboxylic acid (8 mg), DIEA (14 uL) and 50% T3P (45 uL) were added and the mixture was stirred at ambient temperature for 30 minutes. The mixture was purified directly by reverse phase HPLC (C18, 10-100% MeCN/water with 0.1% formic acid) to afford the title compound as a white solid (24 mg, 39% yield). LCMS (ESI) m/z calcd for C₂₁H₃₄N₂O₃S: 394.2. Found: 395.4 (M+1)⁺. ¹H NMR (400 MHz, CDCl₃) δ 7.33-7.30 (m, 1H), 6.77-6.74 (m, 1H), 5.56-5.48 (m, 1H), 4.15-3.90 (m, 3H), 2.71-2.55 (m, 2H), 2.51 (s, 3H), 1.94-1.73 (m, 2H), 1.61-1.52 (m, 1H), 1.51-1.28 (m, 12H), 1.21-0.96 (m, 2H), 0.96-0.90 (m, 6H).

### Example 47: tert-butyl 4-(2-(5-chlorothiophene-2-carboxamido)-2-(5-(pentan-3-yl)-1,2,4-oxadiazol-3-yl)ethyl)piperidine-1-carboxylate

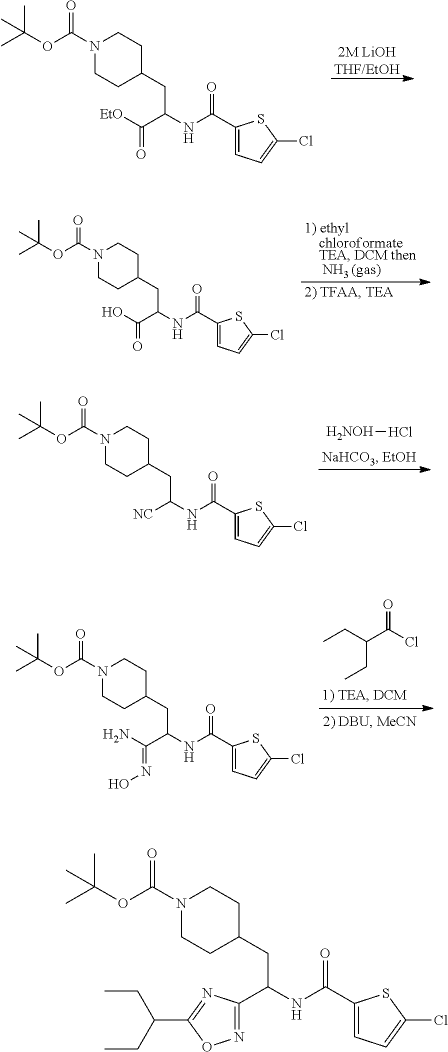

### Step 1: Preparation of 3-(1-(tert-butoxycarbonyl)piperidin-4-yl)-2-(5-chlorothiophene-2-carboxamido)propanoic acid

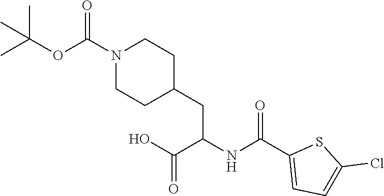 [SMILES: CC(C)(C)OC(=O)N1CCC(CC(NC(=O)c2ccc(Cl)s2)C(=O)O)CC1]

A solution of tert-butyl 4-(2-(5-chlorothiophene-2-carboxamido)-3-ethoxy-3-oxopropyl)piperidine-1-carboxylate (0.577 g, 1.30 mmol) in ethanol (3.24 ml) and THE (9.73 ml) was treated with 2M LiOH (3.24 ml, 6.48 mmol). The mixture was stirred at ambient temperature for one hour and then concentrated. Water was added and the mixture was treated with 1N HCl (3.2 mL) to give a white precipitate. The solids were collected on filter paper (Buchner funnel) under suction filtration and then dried under vacuum to give the desired product as a white solid (0.513 g, 95% yield). LCMS (ESI) m/z calcd for C₁₈H₂₅ClN₂O₅S: 416.1. Found: 417.1 (M+1)⁺. ¹H NMR (400 MHz, DMSO-d₆) δ 12.84-12.55 (m, 1H), 8.74 (d, J=8.2 Hz, 1H), 7.76 (d, J=4.3 Hz, 1H), 7.19 (d, J=3.9 Hz, 1H), 4.43-4.29 (m, 1H), 3.99-3.74 (m, 2H), 2.80-2.52 (m, 2H), 1.77-1.44 (m, 5H), 1.36 (s, 9H), 1.10-0.85 (m, 2H).

### Step 2: Preparation of tert-butyl 4-(2-(5-chlorothiophene-2-carboxamido)-2-cyanoethyl)piperidine-1-carboxylate

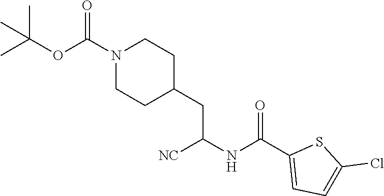 [SMILES: CC(C)(C)OC(=O)N1CCC(CC(C#N)NC(=O)c2ccc(Cl)s2)CC1]

A solution of 3-(1-(tert-butoxycarbonyl)piperidin-4-yl)-2-(5-chlorothiophene-2-carboxamido)propanoic acid (513 mg, 1.230 mmol) and TEA (0.515 mL, 3.69 mmol) in DCM (12 mL) at 0° C. was treated with ethyl chloroformate (0.177 mL, 1.846 mmol) and the mixture was allowed to stir at 0° C. for 45 minutes. The reaction mixture was treated with ammonia gas for 5 minutes (LCMS indicated complete conversion to the primary amide product). The mixture was concentrated to an off-white solid. To a suspension of the crude primary amide product and TEA (0.257 mL, 1.846 mmol) in THE (15 mL) at 0° C. was added TFAA (0.209 mL, 1.477 mmol) and the mixture was allowed to stir at 0° C. for 30 minutes. Additional TFAA (100 uL) was added and the mixture was allowed to stir at ambient temperature for 30 minutes. The mixture was partitioned between EtOAc and saturated NaHCO₃. The aqueous layer was further extracted with EtOAc. The combined extracts were washed with brine, dried over Na₂SO₄, filtered and concentrated. The residue was purified by flash chromatography (silica gel, 0-50% EtOAc/hexanes, gradient elution) to afford the title compound as a viscous pale yellow oil (473 mg, 97% yield). LCMS (ESI) m/z calcd for C₁₈H₂₄ClN₃O₃S: 397.1. Found: 398.2 (M+1)⁺. ¹H NMR (400 MHz, methanol-d₄) δ 7.57 (d, J=4.3 Hz, 1H), 7.06 (d, J=3.9 Hz, 1H), 5.05 (t, J=8.0 Hz, 1H), 4.12-3.99 (m, 2H), 2.85-2.63 (m, 2H), 1.88 (t, J=7.4 Hz, 2H), 1.80-1.60 (m, 3H), 1.43 (s, 9H), 1.23-1.07 (m, 2H).

### Step 3: Preparation of (Z)-tert-butyl 4-(3-amino-2-(5-chlorothiophene-2-carboxamido)-3-(hydroxyimino)propyl)piperidine-1-carboxylate

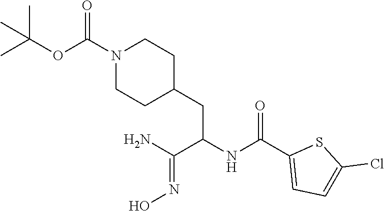 [SMILES: CC(C)(C)OC(=O)N1CCC(CC(NC(=O)c2ccc(Cl)s2)/C(N)=N/O)CC1]

A mixture of tert-butyl 4-(2-(5-chlorothiophene-2-carboxamido)-2-cyanoethyl)piperidine-1-carboxylate (191 mg, 0.480 mmol), hydroxylamine hydrochloride (43.4 mg, 0.624 mmol) and sodium bicarbonate (121 mg, 1.44 mmol) in ethanol (4.0 mL) was heated to 90° C. for 3 hours and then stirred at ambient temperature overnight. Water was added and the mixture was extracted with EtOAc. The extracts were washed with brine, dried over Na₂SO₄, filtered and concentrated to afford the crude product as a white foam (199 mg, 96% yield). LCMS (ESI) m/z calcd for C₁₈H₂₇ClN₄O₄S: 430.1. Found: 431.3 (M+1)⁺.

### Step 4: Preparation of tert-butyl 4-(2-(5-chlorothiophene-2-carboxamido)-2-(5-(pentan-3-yl)-1,2,4-oxadiazol-3-yl)ethyl)piperidine-1-carboxylate

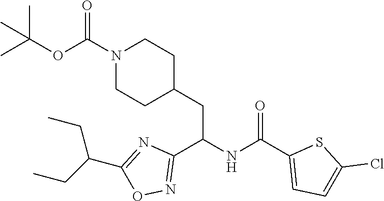 [SMILES: CCC(CC)c1nc(C(CC2CCN(C(=O)OC(C)(C)C)CC2)NC(=O)c2ccc(Cl)s2)no1]

A solution of tert-butyl (Z)-4-(3-amino-2-(5-chlorothiophene-2-carboxamido)-3-(hydroxyimino)propyl)piperidine-1-carboxylate (53 mg, 0.123 mmol) and TEA (0.026 mL, 0.184 mmol) in DCM (1.2 mL) at 0° C. was treated with a solution of 2-ethylbutanoyl chloride (0.020 mL, 0.15 mmol) in DCM (90 uL). The mixture was allowed to stir at ambient temperature for 10 minutes and then concentrated. The residue was suspended in acetonitrile (1.2 mL), treated with DBU (0.022 mL, 0.148 mmol) and the mixture was transferred to a microwave vial. The mixture was subjected to microwave heating at 120° C. for 60 minutes. LCMS indicated approximately 65% conversion to the desired product. The reaction mixture was irradiated in the microwave at 120° C. for an additional 60 minutes. The mixture was concentrated and then purified by flash chromatography (silica gel, 0-50% EtOAc/hexanes, gradient elution) to afford the title compound as a colorless residue (32 mg, 50%). LCMS (ESI) m/z calcd for C₂₄H₃₅ClN₄O₄S: 510.2. Found: 511.3 (M+1)⁺. ¹H NMR (400 MHz, CD₃OD) δ 7.62 (d, J=4.3 Hz, 1H), 7.03 (d, J=4.3 Hz, 1H), 5.40-5.34 (m, 1H), 4.10-4.01 (m, 2H), 2.95-2.86 (m, 1H), 2.82-2.59 (m, 2H), 2.01-1.86 (m, 2H), 1.86-1.68 (m, 6H), 1.65-1.53 (m, 1H), 1.43 (s, 9H), 0.89-0.81 (m, 6H).

### Example 48: tert-butyl 4-(2-(5-(diethylamino)-1,3,4-oxadiazol-2-yl)-2-(5-methylthiophene-2-carboxamido)ethyl)piperidine-1-carboxylate

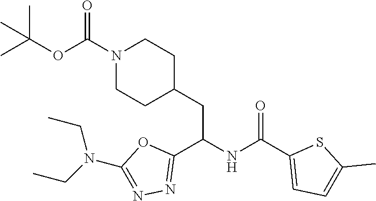 [SMILES: CCN(CC)c1nnc(C(CC2CCN(C(=O)OC(C)(C)C)CC2)NC(=O)c2ccc(C)s2)o1]

The title compound was prepared according to the method described herein for the synthesis of tert-butyl 4-(2-(5-chlorothiophene-2-carboxamido)-2-(5-(diethylamino)-1,3,4-oxadiazol-2-yl)ethyl)piperidine-1-carboxylate employing 5-methylthiophene-2-carboxylic acid in step 3. The product was isolated as a pale yellow solid after flash chromatography (silica gel, 0-100% EtOAc/hexanes, gradient elution). LCMS (ESI) m/z calcd for C₂₄H₃₇N₅O₄S: 491.3. Found: 492.5 (M+1)⁺. ¹H NMR (400 MHz, methanol-d₄) δ 7.56 (d, J=3.5 Hz, 1H), 6.83-6.80 (m, 1H), 5.40-5.32 (m, 1H), 4.05 (d, J=12.9 Hz, 2H), 3.43 (q, J=7.0 Hz, 4H), 2.81-2.61 (m, 2H), 2.51 (s, 3H), 2.01-1.90 (m, 2H), 1.84-1.81 (m, 1H), 1.75-1.69 (m, 1H), 1.68-1.56 (m, 1H), 1.43 (s, 9H), 1.27-1.05 (m, 8H).

### Example 49: tert-butyl 4-(2-(5-ethylthiophene-2-carboxamido)-3-methylbutyl)piperidine-1-carboxylate

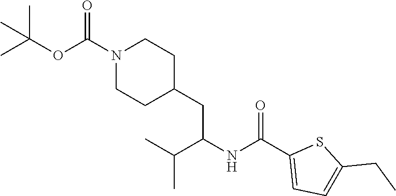 [SMILES: CCc1ccc(C(=O)NC(CC2CCN(C(=O)OC(C)(C)C)CC2)C(C)C)s1]

The title compound was prepared according to the method described herein for the synthesis of tert-butyl 4-(3-methyl-2-(5-methylthiophene-2-carboxamido)butyl)piperidine-1-carboxylate, employing 5-ethylthiophene-2-carboxylic acid in step 3. The product was isolated as a white solid after reverse phase HPLC (C18, 10-100% MeCN/water with 0.1% formic acid) purification. LCMS (ESI) m/z calcd for C₂₂H₃₆N₂O₃S: 408.2. Found: 409.4 (M+1)⁺. ¹H NMR (400 MHz, CDCl₃) δ 7.33 (d, J=3.5 Hz, 1H), 6.77 (d, J=3.9 Hz, 1H), 5.53 (d, J=9.8 Hz, 1H), 4.17-3.87 (m, 3H), 2.86 (q, J=7.5 Hz, 2H), 2.71-2.56 (m, 2H), 1.93-1.85 (m, 1H), 1.83-1.73 (m, 1H), 1.62-1.52 (m, 1H), 1.51-1.27 (m, 15H), 1.21-0.87 (m, 8H).

### Example 50: tert-butyl 4-(2-(5-(cyclopropyl(ethyl)amino)-1,3,4-oxadiazol-2-yl)-2-(5-methylthiophene-2-carboxamido)ethyl)piperidine-1-carboxylate

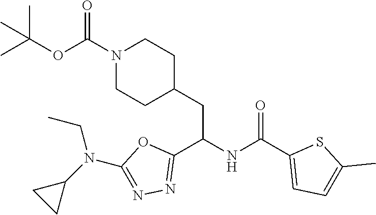 [SMILES: CCN(c1nnc(C(CC2CCN(C(=O)OC(C)(C)C)CC2)NC(=O)c2ccc(C)s2)o1)C1CC1]

The title compound was prepared according to the method described herein for the synthesis of tert-butyl 4-(2-(5-chlorothiophene-2-carboxamido)-2-(5-(diethylamino)-1,3,4-oxadiazol-2-yl)ethyl)piperidine-1-carboxylate, employing 5-methylthiophene-2-carboxylic acid in step 3 and N-ethylcyclopropanamine in step 6. The product was isolated as a colorless residue after reverse phase HPLC (C18, 10-100% MeCN/water with 0.1% formic acid) purification. LCMS (ESI) m/z calcd for C₂₅H₃₇N₅O₄S: 503.3. Found: 504.4 (M+1)⁺. ¹H NMR (400 MHz, methanol-d₄) δ 7.56 (d, J=3.9 Hz, 1H), 6.85-6.78 (m, 1H), 5.39-5.33 (m, 1H), 4.10-4.01 (m, 2H), 3.46 (q, J=7.2 Hz, 2H), 2.85-2.60 (m, 3H), 2.50 (s, 3H), 2.04-1.89 (m, 2H), 1.86-1.78 (m, 1H), 1.77-1.70 (m, 1H), 1.68-1.57 (m, 1H), 1.43 (s, 9H), 1.26-1.05 (m, 5H), 0.87-0.78 (m, 2H), 0.74-0.65 (m, 2H).

### Example 51: phenyl 4-(2-(5-methylthiophene-2-carboxamido)-2-phenylethyl)piperidine-1-carboxylate

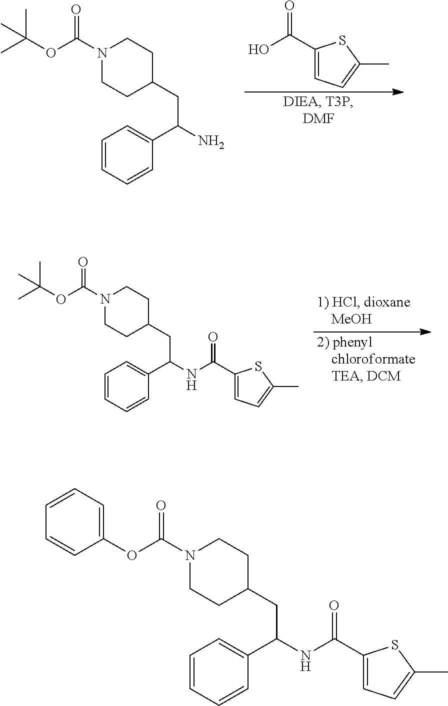

### Step 1: Preparation of tert-butyl 4-(2-(5-methylthiophene-2-carboxamido)-2-phenylethyl)piperidine-1-carboxylate

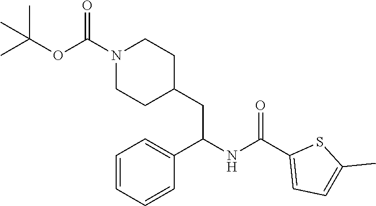 [SMILES: Cc1ccc(C(=O)NC(CC2CCN(C(=O)OC(C)(C)C)CC2)c2ccccc2)s1]

A solution of tert-butyl 4-(2-amino-2-phenylethyl)piperidine-1-carboxylate (798 mg, 2.62 mmol), 5-methylthiophene-2-carboxylic acid (373 mg, 2.62 mmol) and DIEA (0.687 mL, 3.93 mmol) in DMF (15 mL) was treated with 50% T3P/EtOAc (2.497 mL, 4.19 mmol) and the mixture was stirred at ambient temperature overnight. Saturated NaHCO₃ was added and the mixture was extracted with EtOAc. The extracts were washed with water, then brine, dried over Na₂SO₄, filtered and concentrated. The residue was purified by flash chromatography (silica gel, 0-50% EtOAc/hexanes, gradient elution) to afford the title compound a white foam (350 mg, 31% yield). LCMS (ESI) m/z calcd for C₂₄H₃₂N₂O₃S: 428.2. Found: 429.4 (M+1)⁺. ¹H NMR (400 MHz, methanol-d₄) δ 8.57 (d, J=8.6 Hz, 1H), 7.56 (d, J=3.5 Hz, 1H), 7.39-7.27 (m, 4H), 7.25-7.18 (m, 1H), 6.80 (d, J=3.1 Hz, 1H), 5.22-5.13 (m, 1H), 4.04 (d, J=13.3 Hz, 2H), 2.78-2.59 (m, 2H), 2.49 (s, 3H), 1.96-1.86 (m, 1H), 1.85-1.78 (m, 1H), 1.77-1.65 (m, 2H), 1.59-1.48 (m, 1H), 1.43 (s, 9H), 1.22-1.05 (m, 2H).

### Step 2: Preparation of phenyl 4-(2-(5-methylthiophene-2-carboxamido)-2-phenylethyl)piperidine-1-carboxylate

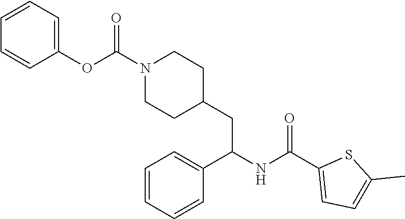 [SMILES: Cc1ccc(C(=O)NC(CC2CCN(C(=O)Oc3ccccc3)CC2)c2ccccc2)s1]

A solution of tert-butyl 4-(2-(5-methylthiophene-2-carboxamido)-2-phenylethyl)piperidine-1-carboxylate (70 mg, 0.163 mmol) in methanol (0.75 mL) was treated with 4M HCl/dioxane (1.50 mL, 6.00 mmol). The mixture was stirred for 20 minutes at ambient temperature and then concentrated to give the intermediate amine hydrochloride as a pale yellow residue (78 mg). To a suspension of the intermediate and TEA (0.091 mL, 0.653 mmol) in DCM (3.0 mL) at 0° C. was added a solution of phenyl chloroformate (0.025 mL, 0.20 mmol) in DCM (450 uL) and the mixture stirred at ambient temperature for 20 minutes. Saturated NaHCO₃ was added and the mixture was extracted with DCM. The combined organic phases were dried over MgSO₄, filtered and concentrated. The residue was purified by flash chromatography (silica gel, 0-50% EtOAc/hexanes, gradient elution) to afford the title compound as a white solid (54 mg, 73%). LCMS (ESI) m/z calcd for C₂₆H₂₈N₂O₃S: 448.2. Found: 449.3 (M+1)⁺. ¹H NMR (400 MHz, methanol-d₄) δ 8.60 (d, J=8.6 Hz, 1H), 7.58 (d, J=3.9 Hz, 1H), 7.42-7.29 (m, 6H), 7.26-7.17 (m, 2H), 7.08-7.04 (m, 2H), 6.82-6.79 (m, 1H), 5.27-5.16 (m, 1H), 4.35-4.22 (m, 1H), 4.19-4.08 (m, 1H), 3.06-2.74 (m, 2H), 2.49 (s, 3H), 2.02-1.55 (m, 5H), 1.41-1.18 (m, 2H).

### Example 52: tert-butyl 4-(2-(5-(ethyl(methyl)amino)-1,3,4-oxadiazol-2-yl)-2-(5-methylthiophene-2-carboxamido)ethyl)piperidine-1-carboxylate

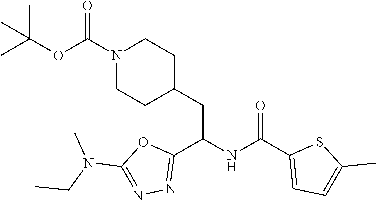 [SMILES: CCN(C)c1nnc(C(CC2CCN(C(=O)OC(C)(C)C)CC2)NC(=O)c2ccc(C)s2)o1]

The title compound was prepared according to the method described herein for the synthesis of tert-butyl 4-(2-(5-chlorothiophene-2-carboxamido)-2-(5-(diethylamino)-1,3,4-oxadiazol-2-yl)ethyl)piperidine-1-carboxylate, employing 5-methylthiophene-2-carboxylic acid in step 3 and N-methylethanamine in step 6. The product was isolated as a colorless residue after flash chromatography (silica gel, 0-100% EtOAc/hexanes, gradient elution). LCMS (ESI) m/z calcd for C₂₃H₃₅N₅O₄S: 477.2. Found: 478.4 (M+1)⁺. ¹H NMR (400 MHz, methanol-d₄) δ 7.56 (d, J=3.9 Hz, 1H), 6.85-6.79 (m, 1H), 5.39-5.33 (m, 1H), 4.10-4.00 (m, 2H), 3.43 (q, J=7.0 Hz, 2H), 3.05-3.01 (m, 3H), 2.82-2.59 (m, 2H), 2.51 (s, 3H), 2.02-1.89 (m, 2H), 1.86-1.78 (m, 1H), 1.76-1.69 (m, 1H), 1.68-1.55 (m, 1H), 1.49-1.37 (m, 9H), 1.26-1.05 (m, 5H).

### Example 53: phenyl 4-(2-(5-chlorothiophene-2-carboxamido)-2-(5-(diethylamino)-1,3,4-oxadiazol-2-yl)ethyl)piperidine-1-carboxylate

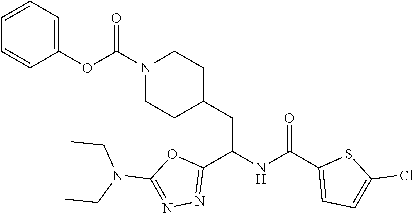 [SMILES: CCN(CC)c1nnc(C(CC2CCN(C(=O)Oc3ccccc3)CC2)NC(=O)c2ccc(Cl)s2)o1]

A solution of tert-butyl 4-(2-(5-chlorothiophene-2-carboxamido)-2-(5-(diethylamino)-1,3,4-oxadiazol-2-yl)ethyl)piperidine-1-carboxylate (60 mg, 0.117 mmol) in methanol (0.5 mL) was treated with 4N HCl/dioxane (1.00 mL, 4.00 mmol) at ambient temperature. The mixture was stirred at ambient temperature for 10 minutes and then concentrated to afford the amine hydrochloride intermediate as a pale yellow residue. An ice cold suspension of the intermediate and TEA (0.065 mL, 0.469 mmol) in DCM (2.0 mL) was treated with a solution of phenyl chloroformate (0.018 mL, 0.141 mmol) in DCM (0.31 mL). The cooling bath was removed and the mixture was stirred at ambient temperature for 45 minutes. The mixture was partitioned between DCM and saturated NaHCO₃ and the phases were separated. The aqueous phase was extracted with DCM and the combined organic phases were dried over MgSO4, filtered and concentrated. The residue was purified by reverse phase HPLC (C18, 10-100% MeCN/water with 0.1% formic acid) followed by flash chromatography (silica gel, 30-100% EtOAc/hexanes, gradient elution) to afford the title compound as a colorless residue (12 mg, 18% yield). LCMS (ESI) m/z calcd for C₂₅H₃₀ClN₅O₄S: 531.2. Found: 532.4 (M+1)⁺. ¹H NMR (400 MHz, CDCl₃) δ 7.78-7.47 (m, 1H), 7.42-7.30 (m, 3H), 7.22-7.14 (m, 1H), 7.11-7.06 (m, 2H), 6.84 (d, J=3.9 Hz, 1H), 5.51-5.43 (m, 1H), 4.34-4.18 (m, 2H), 3.43 (q, J=7.0 Hz, 4H), 3.08-2.63 (m, 2H), 2.00-1.84 (m, 3H), 1.81-1.69 (m, 2H), 1.31-1.16 (m, 8H).

Examples 54-245 were prepared using methods similar to those described herein for examples 1-53.

### Example 54: tert-butyl 4-(2-(4-fluorobenzamido)-2-(6-methoxypyridin-3-yl)ethyl)piperidine-1-carboxylate

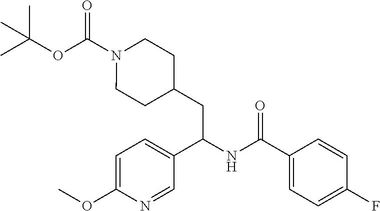 [SMILES: COc1ccc(C(CC2CCN(C(=O)OC(C)(C)C)CC2)NC(=O)c2ccc(F)cc2)cn1]

### Example 55: isopropyl 4-(2-(4-fluorobenzamido)-2-(6-methoxypyridin-3-yl)ethyl)piperidine-1-carboxylate

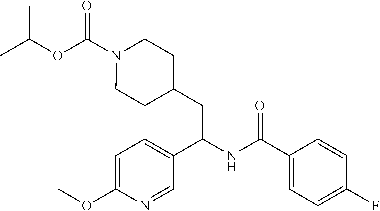 [SMILES: COc1ccc(C(CC2CCN(C(=O)OC(C)C)CC2)NC(=O)c2ccc(F)cc2)cn1]

### Example 56: isobutyl 4-(2-(4-fluorobenzamido)-2-(6-methoxypyridin-3-yl)ethyl)piperidine-1-carboxylate

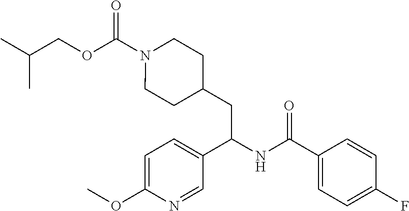 [SMILES: COc1ccc(C(CC2CCN(C(=O)OCC(C)C)CC2)NC(=O)c2ccc(F)cc2)cn1]

### Example 57: tert-butyl 4-(2-(4-bromobenzamido)-2-phenylethyl)piperidine-1-carboxylate

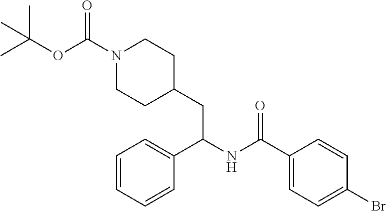 [SMILES: CC(C)(C)OC(=O)N1CCC(CC(NC(=O)c2ccc(Br)cc2)c2ccccc2)CC1]

### Example 58: tert-butyl 4-(2-(5-fluorothiophene-2-carboxamido)-2-phenylethyl)piperidine-1-carboxylate

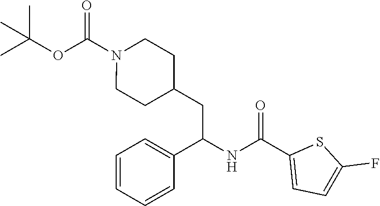 [SMILES: CC(C)(C)OC(=O)N1CCC(CC(NC(=O)c2ccc(F)s2)c2ccccc2)CC1]

### Example 59: tert-butyl 4-(2-cyclopentyl-2-(5-methylthiophene-2-carboxamido)ethyl)piperidine-1-carboxylate

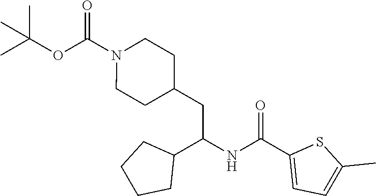 [SMILES: Cc1ccc(C(=O)NC(CC2CCN(C(=O)OC(C)(C)C)CC2)C2CCCC2)s1]

### Example 60: tert-butyl 4-(2-(5-ethylthiophene-2-carboxamido)-4-methylpentyl)piperidine-1-carboxylate

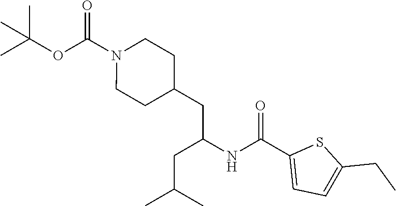 [SMILES: CCc1ccc(C(=O)NC(CC(C)C)CC2CCN(C(=O)OC(C)(C)C)CC2)s1]

### Example 61: cyclobutyl 4-(2-(4-chlorobenzamido)-2-phenylethyl)piperidine-1-carboxylate

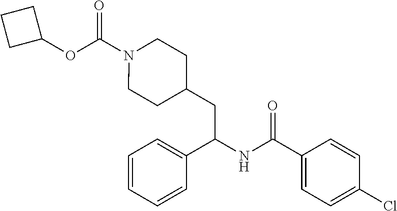 [SMILES: O=C(NC(CC1CCN(C(=O)OC2CCC2)CC1)c1ccccc1)c1ccc(Cl)cc1]

### Example 62: prop-2-yn-1-yl 4-(2-(4-chlorobenzamido)-2-phenylethyl)piperidine-1-carboxylate

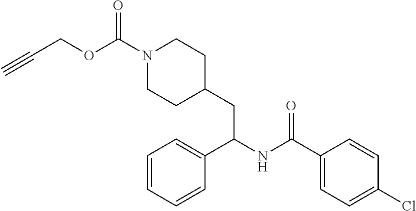 [SMILES: C#CCOC(=O)N1CCC(CC(NC(=O)c2ccc(Cl)cc2)c2ccccc2)CC1]

### Example 63: (S)-tert-butyl 4-(2-(4-bromobenzamido)-2-cyclopropylethyl)piperidine-1-carboxylate

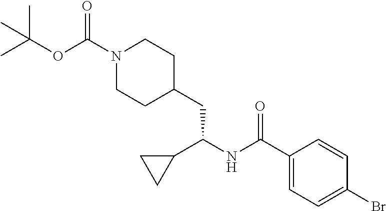 [SMILES: CC(C)(C)OC(=O)N1CCC(C[C@H](NC(=O)c2ccc(Br)cc2)C2CC2)CC1]

### Example 64: tert-butyl 4-(2-(5-chlorothiophene-2-carboxamido)-2-cyclohexylethyl)piperidine-1-carboxylate

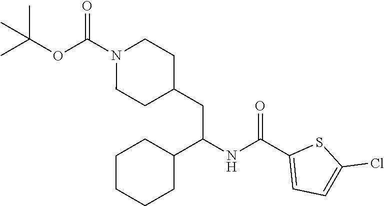 [SMILES: CC(C)(C)OC(=O)N1CCC(CC(NC(=O)c2ccc(Cl)s2)C2CCCCC2)CC1]

### Example 65: (S)-tert-butyl 4-(2-(4-chlorobenzamido)-2-phenylethyl)piperidine-1-carboxylate

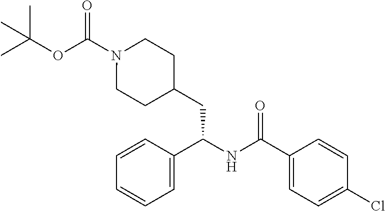 [SMILES: CC(C)(C)OC(=O)N1CCC(C[C@H](NC(=O)c2ccc(Cl)cc2)c2ccccc2)CC1]

### Example 66: (S)-ethyl 4-(2-(5-ethylthiophene-2-carboxamido)-2-phenylethyl)piperidine-1-carboxylate

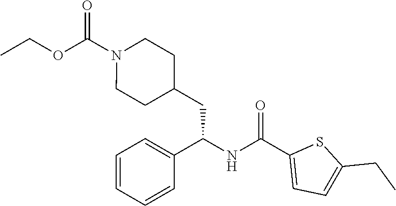 [SMILES: CCOC(=O)N1CCC(C[C@H](NC(=O)c2ccc(CC)s2)c2ccccc2)CC1]

### Example 67: phenyl 4-(2-(4-chlorobenzamido)-4-methylpentyl)piperidine-1-carboxylate

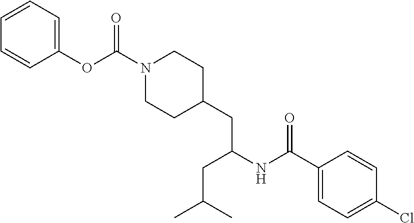 [SMILES: CC(C)CC(CC1CCN(C(=O)Oc2ccccc2)CC1)NC(=O)c1ccc(Cl)cc1]

### Example 68: tert-butyl 4-(2-(4-chlorobenzamido)-2-phenylethyl)piperidine-1-carboxylate

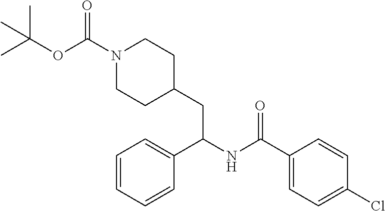 [SMILES: CC(C)(C)OC(=O)N1CCC(CC(NC(=O)c2ccc(Cl)cc2)c2ccccc2)CC1]

### Example 69: tert-butyl 4-(2-(5-methylthiophene-2-carboxamido)-2-phenylethyl)piperidine-1-carboxylate

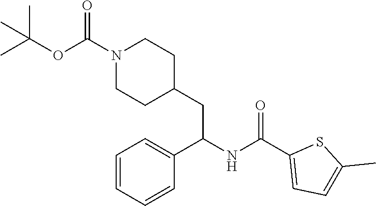 [SMILES: Cc1ccc(C(=O)NC(CC2CCN(C(=O)OC(C)(C)C)CC2)c2ccccc2)s1]

### Example 70: (R)-tert-butyl 4-(2-(5-chlorothiophene-2-carboxamido)-2-(6-methoxypyridin-3-yl)ethyl)piperidine-1-carboxylate

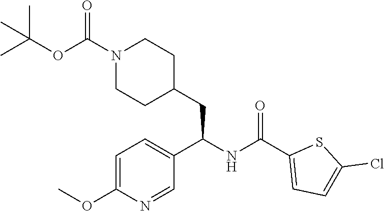 [SMILES: COc1ccc([C@@H](CC2CCN(C(=O)OC(C)(C)C)CC2)NC(=O)c2ccc(Cl)s2)cn1]

### Example 71: tert-butyl 4-(2-(4-bromo-3-fluorobenzamido)-2-phenylethyl)piperidine-1-carboxylate

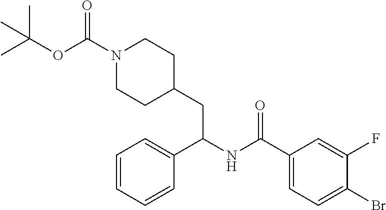 [SMILES: CC(C)(C)OC(=O)N1CCC(CC(NC(=O)c2ccc(Br)c(F)c2)c2ccccc2)CC1]

### Example 72: tert-butyl 4-(2-(4-iodobenzamido)-2-phenylethyl)piperidine-1-carboxylate

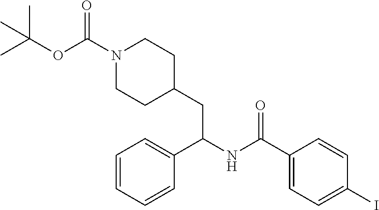 [SMILES: CC(C)(C)OC(=O)N1CCC(CC(NC(=O)c2ccc(I)cc2)c2ccccc2)CC1]

### Example 73: tert-butyl 4-(2-(5-chlorothiophene-2-carboxamido)-2-(5-(dimethylamino)-1,3,4-oxadiazol-2-yl)ethyl)piperidine-1-carboxylate

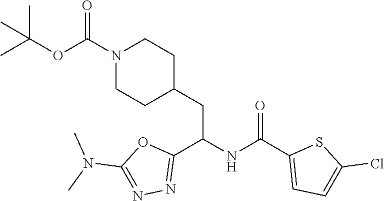 [SMILES: CN(C)c1nnc(C(CC2CCN(C(=O)OC(C)(C)C)CC2)NC(=O)c2ccc(Cl)s2)o1]

### Example 74: tert-butyl 4-(2-(4-bromobenzamido)-3-methylbutyl)piperidine-1-carboxylate

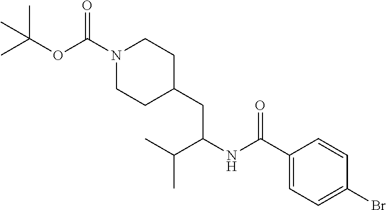 [SMILES: CC(C)C(CC1CCN(C(=O)OC(C)(C)C)CC1)NC(=O)c1ccc(Br)cc1]

### Example 75: tert-butyl 4-(2-(4-chlorobenzamido)-4-methylpentyl)piperidine-1-carboxylate

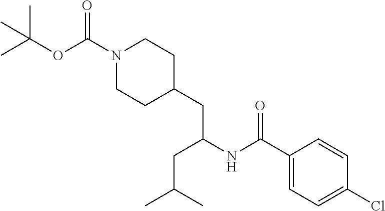 [SMILES: CC(C)CC(CC1CCN(C(=O)OC(C)(C)C)CC1)NC(=O)c1ccc(Cl)cc1]

### Example 76: tert-butyl 4-(2-(4-fluorobenzamido)-4-methylpentyl)piperidine-1-carboxylate

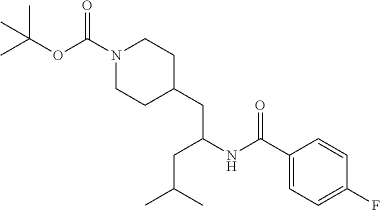 [SMILES: CC(C)CC(CC1CCN(C(=O)OC(C)(C)C)CC1)NC(=O)c1ccc(F)cc1]

### Example 77: cyclopropyl 4-(2-(4-chlorobenzamido)-2-phenylethyl)piperidine-1-carboxylate

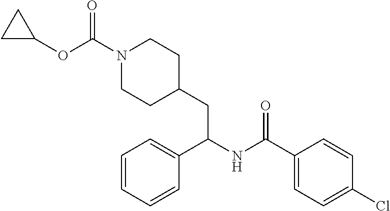 [SMILES: O=C(NC(CC1CCN(C(=O)OC2CC2)CC1)c1ccccc1)c1ccc(Cl)cc1]

### Example 78: tert-butyl 4-(2-cyclopentyl-2-(5-ethylthiophene-2-carboxamido)ethyl)piperidine-1-carboxylate

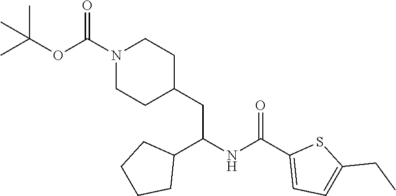 [SMILES: CCc1ccc(C(=O)NC(CC2CCN(C(=O)OC(C)(C)C)CC2)C2CCCC2)s1]

### Example 79: tert-butyl 4-(2-(5-fluorothiophene-2-carboxamido)-4-methylpentyl)piperidine-1-carboxylate

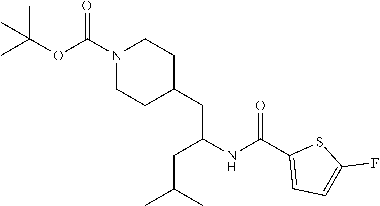 [SMILES: CC(C)CC(CC1CCN(C(=O)OC(C)(C)C)CC1)NC(=O)c1ccc(F)s1]

### Example 80: tert-butyl 4-(2-cyclohexyl-2-(5-methylthiophene-2-carboxamido)ethyl)piperidine-1-carboxylate

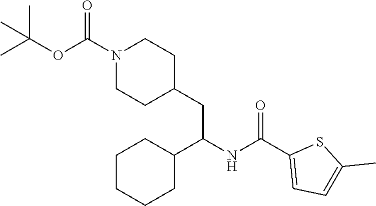 [SMILES: Cc1ccc(C(=O)NC(CC2CCN(C(=O)OC(C)(C)C)CC2)C2CCCCC2)s1]

### Example 81: (S)-ethyl 4-(2-(4-chlorobenzamido)-2-phenylethyl)piperidine-1-carboxylate

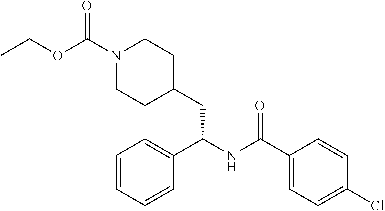 [SMILES: CCOC(=O)N1CCC(C[C@H](NC(=O)c2ccc(Cl)cc2)c2ccccc2)CC1]

### Example 82: tert-butyl 4-(2-(5-(ethyl(2-methoxyethyl)amino)-1,3,4-oxadiazol-2-yl)-2-(5-methylthiophene-2-carboxamido)ethyl)piperidine-1-carboxylate

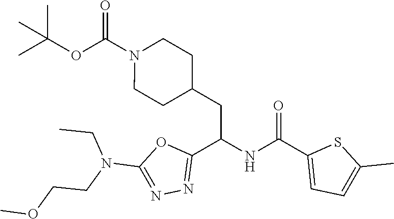

### Example 83: (S)-tert-butyl 4-(4-(benzylamino)-2-(5-chlorothiophene-2-carboxamido)butyl)piperidine-1-carboxylate

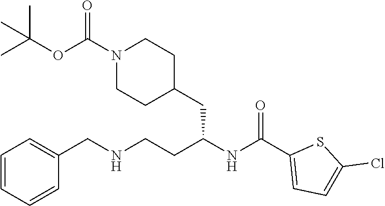 [SMILES: CC(C)(C)OC(=O)N1CCC(C[C@@H](CCNCc2ccccc2)NC(=O)c2ccc(Cl)s2)CC1]

### Example 84: tert-butyl 4-(2-(4-chloro-3-fluorobenzamido)-2-phenylethyl)piperidine-1-carboxylate

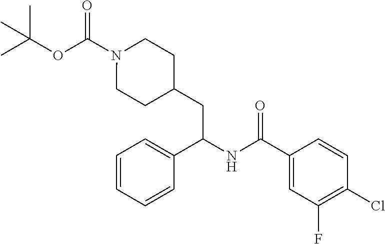 [SMILES: CC(C)(C)OC(=O)N1CCC(CC(NC(=O)c2ccc(Cl)c(F)c2)c2ccccc2)CC1]

### Example 85: ethyl 4-(2-(4-fluorobenzamido)-2-(6-methoxypyridin-3-yl)ethyl)piperidine-1-carboxylate

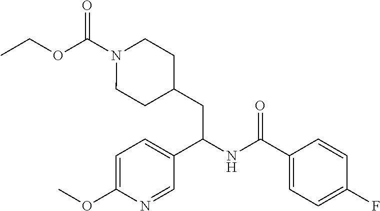 [SMILES: CCOC(=O)N1CCC(CC(NC(=O)c2ccc(F)cc2)c2ccc(OC)nc2)CC1]

### Example 86: tert-butyl 4-(2-phenyl-2-(4-(prop-2-yn-1-yloxy)benzamido)ethyl)piperidine-1-carboxylate

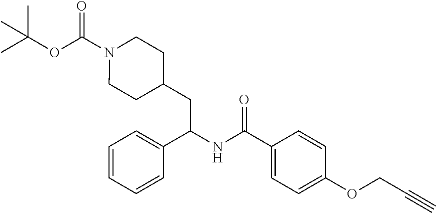 [SMILES: C#CCOc1ccc(C(=O)NC(CC2CCN(C(=O)OC(C)(C)C)CC2)c2ccccc2)cc1]

### Example 87: tert-butyl 4-(2-phenyl-2-(5-propylthiophene-2-carboxamido)ethyl)piperidine-1-carboxylate

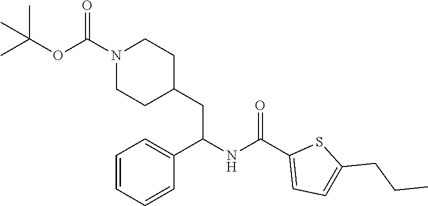 [SMILES: CCCc1ccc(C(=O)NC(CC2CCN(C(=O)OC(C)(C)C)CC2)c2ccccc2)s1]

### Example 88: tert-butyl 4-(2-(4-chlorobenzamido)-3-methylbutyl)piperidine-1-carboxylate

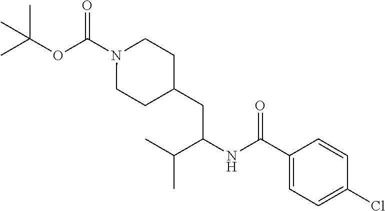 [SMILES: CC(C)C(CC1CCN(C(=O)OC(C)(C)C)CC1)NC(=O)c1ccc(Cl)cc1]

### Example 89: propyl 4-(2-(4-chlorobenzamido)-2-phenylethyl)piperidine-1-carboxylate

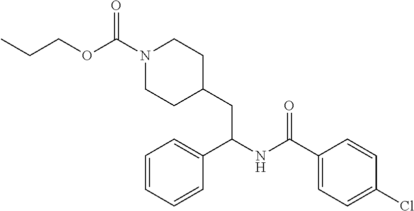 [SMILES: CCCOC(=O)N1CCC(CC(NC(=O)c2ccc(Cl)cc2)c2ccccc2)CC1]

### Example 90: (S)-tert-butyl 4-(2-(4-chlorobenzamido)-2-cyclopropylethyl)piperidine-1-carboxylate

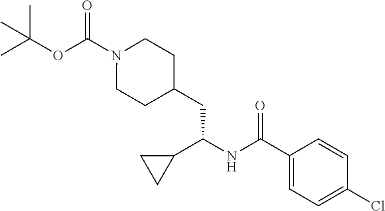 [SMILES: CC(C)(C)OC(=O)N1CCC(C[C@H](NC(=O)c2ccc(Cl)cc2)C2CC2)CC1]

### Example 91: tert-butyl 4-(2-(4-bromobenzamido)-2-cyclohexylethyl)piperidine-1-carboxylate

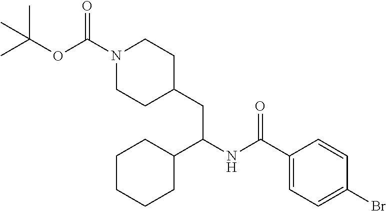 [SMILES: CC(C)(C)OC(=O)N1CCC(CC(NC(=O)c2ccc(Br)cc2)C2CCCCC2)CC1]

### Example 92: tert-butyl 4-(2-cyclohexyl-2-(5-fluorothiophene-2-carboxamido)ethyl)piperidine-1-carboxylate

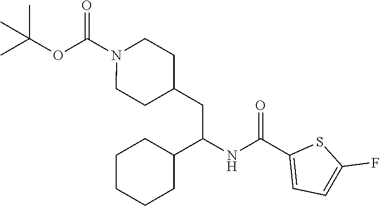 [SMILES: CC(C)(C)OC(=O)N1CCC(CC(NC(=O)c2ccc(F)s2)C2CCCCC2)CC1]

### Example 93: N-(2-(1-(2,2-difluoro-2-phenylacetyl)piperidin-4-yl)-1-phenylethyl)-5-methylthiophene-2-carboxamide

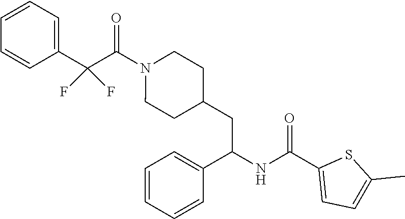 [SMILES: Cc1ccc(C(=O)NC(CC2CCN(C(=O)C(F)(F)c3ccccc3)CC2)c2ccccc2)s1]

### Example 94: 5-methyl-N-(1-phenyl-2-(1-(2,2,2-trifluoroacetyl)piperidin-4-yl)ethyl)thiophene-2-carboxamide

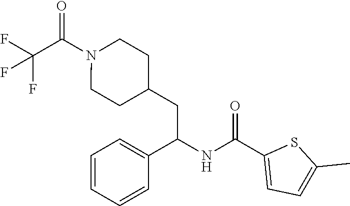 [SMILES: Cc1ccc(C(=O)NC(CC2CCN(C(=O)C(F)(F)F)CC2)c2ccccc2)s1]

### Example 95: N-(2-(1-(2,2-difluorobutanoyl)piperidin-4-yl)-1-phenylethyl)-5-methylthiophene-2-carboxamide

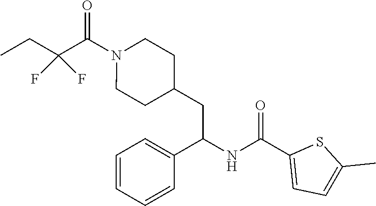 [SMILES: CCC(F)(F)C(=O)N1CCC(CC(NC(=O)c2ccc(C)s2)c2ccccc2)CC1]

### Example 96: tert-butyl 4-(2-(5-(butyl(ethyl)amino)-1,3,4-oxadiazol-2-yl)-2-(5-methylthiophene-2-carboxamido)ethyl)piperidine-1-carboxylate

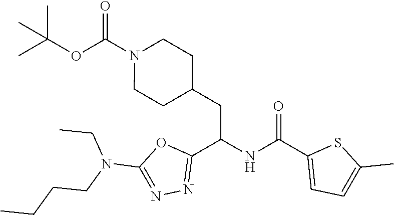 [SMILES: CCCCN(CC)c1nnc(C(CC2CCN(C(=O)OC(C)(C)C)CC2)NC(=O)c2ccc(C)s2)o1]

### Example 97: tert-butyl 4-(2-(4-cyclopropylbenzamido)-2-phenylethyl)piperidine-1-carboxylate

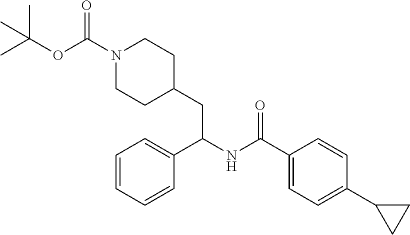 [SMILES: CC(C)(C)OC(=O)N1CCC(CC(NC(=O)c2ccc(C3CC3)cc2)c2ccccc2)CC1]

### Example 98: tert-butyl 4-(2-(5-cyclopropylthiophene-2-carboxamido)-2-phenylethyl)piperidine-1-carboxylate

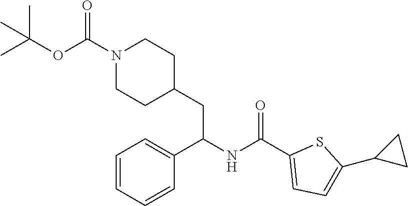 [SMILES: CC(C)(C)OC(=O)N1CCC(CC(NC(=O)c2ccc(C3CC3)s2)c2ccccc2)CC1]

### Example 99: tert-butyl 4-(2-(5-chlorothiophene-2-carboxamido)-3-methylbutyl)piperidine-1-carboxylate

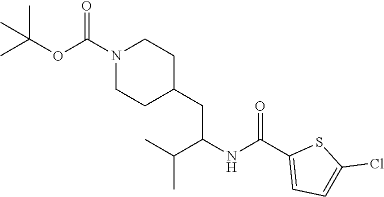 [SMILES: CC(C)C(CC1CCN(C(=O)OC(C)(C)C)CC1)NC(=O)c1ccc(Cl)s1]

### Example 100: cyclopentyl 4-(2-(4-chlorobenzamido)-2-phenylethyl)piperidine-1-carboxylate

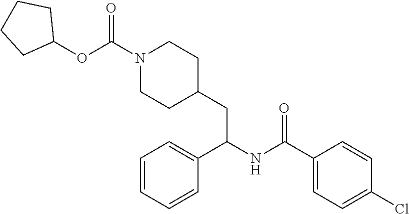 [SMILES: O=C(NC(CC1CCN(C(=O)OC2CCCC2)CC1)c1ccccc1)c1ccc(Cl)cc1]

### Example 101: tert-butyl 4-(2-(3-fluoro-4-iodobenzamido)-2-phenylethyl)piperidine-1-carboxylate

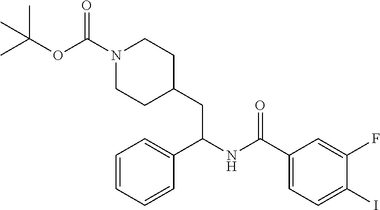 [SMILES: CC(C)(C)OC(=O)N1CCC(CC(NC(=O)c2ccc(I)c(F)c2)c2ccccc2)CC1]

### Example 102: ethyl 4-(2-(4-chlorobenzamido)-2-phenylethyl)piperidine-1-carboxylate

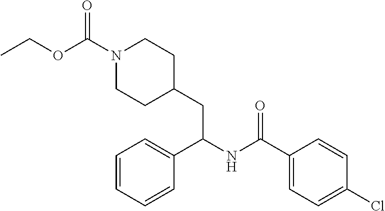 [SMILES: CCOC(=O)N1CCC(CC(NC(=O)c2ccc(Cl)cc2)c2ccccc2)CC1]

### Example 103: isopropyl 4-(2-(4-chlorobenzamido)-2-phenylethyl)piperidine-1-carboxylate

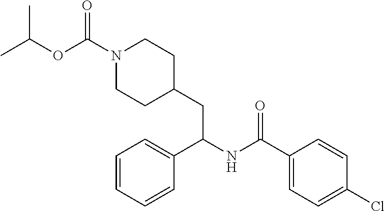 [SMILES: CC(C)OC(=O)N1CCC(CC(NC(=O)c2ccc(Cl)cc2)c2ccccc2)CC1]

### Example 104: phenyl 4-(2-(4-chlorobenzamido)-2-phenylethyl)piperidine-1-carboxylate

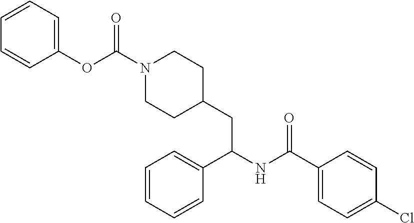 [SMILES: O=C(NC(CC1CCN(C(=O)Oc2ccccc2)CC1)c1ccccc1)c1ccc(Cl)cc1]

### Example 105: cyclopropylmethyl 4-(2-(4-chlorobenzamido)-2-phenylethyl)piperidine-1-carboxylate

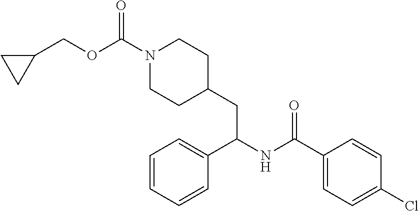 [SMILES: O=C(NC(CC1CCN(C(=O)OCC2CC2)CC1)c1ccccc1)c1ccc(Cl)cc1]

### Example 106: tert-butyl 4-(2-cyclohexyl-2-(5-ethylthiophene-2-carboxamido)ethyl)piperidine-1-carboxylate

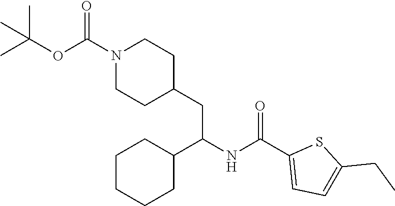 [SMILES: CCc1ccc(C(=O)NC(CC2CCN(C(=O)OC(C)(C)C)CC2)C2CCCCC2)s1]

### Example 107: tert-butyl 4-(2-(4-fluorobenzamido)-2-phenylethyl)piperidine-1-carboxylate

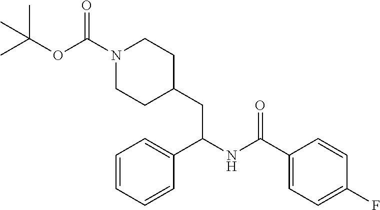 [SMILES: CC(C)(C)OC(=O)N1CCC(CC(NC(=O)c2ccc(F)cc2)c2ccccc2)CC1]

### Example 108: ethyl 4-(2-cyclopropyl-2-(4-fluorobenzamido)ethyl)piperidine-1-carboxylate

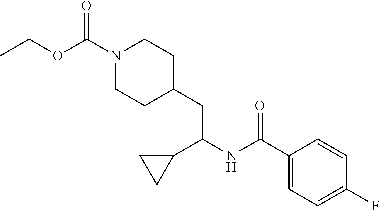 [SMILES: CCOC(=O)N1CCC(CC(NC(=O)c2ccc(F)cc2)C2CC2)CC1]

### Example 109: tert-butyl 4-(2-(3-fluoro-4-methoxybenzamido)-2-phenylethyl)piperidine-1-carboxylate

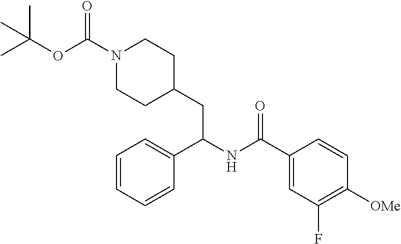 [SMILES: COc1ccc(C(=O)NC(CC2CCN(C(=O)OC(C)(C)C)CC2)c2ccccc2)cc1F]

### Example 110: tert-butyl 4-(2-phenyl-2-(4-(trifluoromethyl)benzamido)ethyl)piperidine-1-carboxylate

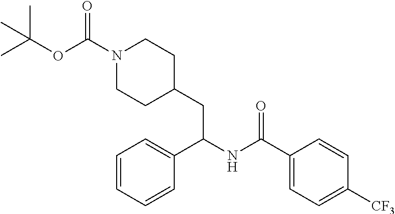 [SMILES: CC(C)(C)OC(=O)N1CCC(CC(NC(=O)c2ccc(C(F)(F)F)cc2)c2ccccc2)CC1]

### Example 111: tert-butyl 4-(2-(6-methoxypyridin-3-yl)-2-(4-(methylthio)benzamido)ethyl)piperidine-1-carboxylate

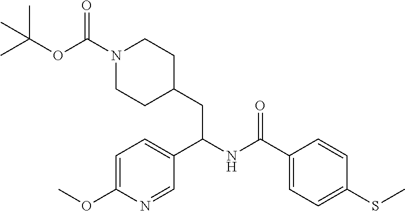 [SMILES: COc1ccc(C(CC2CCN(C(=O)OC(C)(C)C)CC2)NC(=O)c2ccc(SC)cc2)cn1]

### Example 112: tert-butyl 4-(2-(5-(diethylamino)-1,3,4-oxadiazol-2-yl)-2-(4-fluorobenzamido)ethyl)piperidine-1-carboxylate

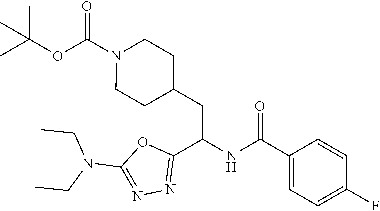 [SMILES: CCN(CC)c1nnc(C(CC2CCN(C(=O)OC(C)(C)C)CC2)NC(=O)c2ccc(F)cc2)o1]

### Example 113: tert-butyl 4-(2-(5-chlorothiophene-2-carboxamido)-2-(5-((2-methoxyethyl)(methyl)amino)-1,3,4-oxadiazol-2-yl)ethyl)piperidine-1-carboxylate

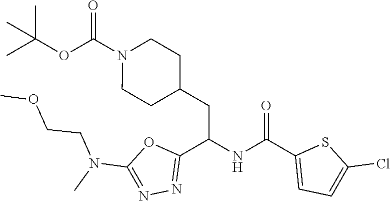 [SMILES: COCCN(C)c1nnc(C(CC2CCN(C(=O)OC(C)(C)C)CC2)NC(=O)c2ccc(Cl)s2)o1]

### Example 114: tert-butyl 4-(2-(3-fluoro-4-(prop-2-yn-1-yloxy)benzamido)-2-phenylethyl)piperidine-1-carboxylate

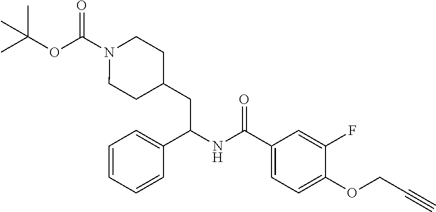 [SMILES: C#CCOc1ccc(C(=O)NC(CC2CCN(C(=O)OC(C)(C)C)CC2)c2ccccc2)cc1F]

### Example 115: tert-butyl 4-(2-(5-chlorothiophene-2-carboxamido)-2-cyclopentylethyl)piperidine-1-carboxylate

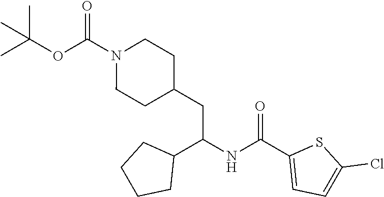 [SMILES: CC(C)(C)OC(=O)N1CCC(CC(NC(=O)c2ccc(Cl)s2)C2CCCC2)CC1]

### Example 116: tert-butyl 4-(2-cyclopentyl-2-(5-fluorothiophene-2-carboxamido)ethyl)piperidine-1-carboxylate

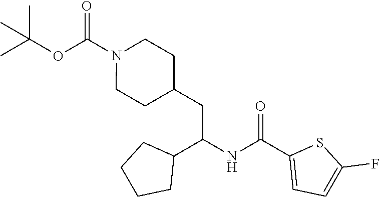 [SMILES: CC(C)(C)OC(=O)N1CCC(CC(NC(=O)c2ccc(F)s2)C2CCCC2)CC1]

### Example 117: benzyl 4-(2-(4-chlorobenzamido)-2-phenylethyl)piperidine-1-carboxylate

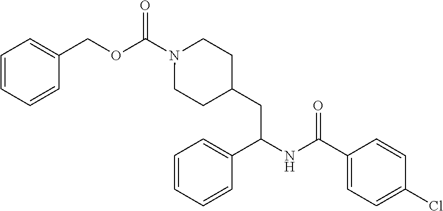 [SMILES: O=C(NC(CC1CCN(C(=O)OCc2ccccc2)CC1)c1ccccc1)c1ccc(Cl)cc1]

### Example 118: tert-butyl 4-(2-(4-ethylbenzamido)-2-phenylethyl)piperidine-1-carboxylate

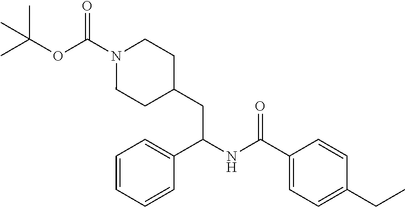 [SMILES: CCc1ccc(C(=O)NC(CC2CCN(C(=O)OC(C)(C)C)CC2)c2ccccc2)cc1]

### Example 119: tert-butyl 4-(2-phenyl-2-(4-vinylbenzamido)ethyl)piperidine-1-carboxylate

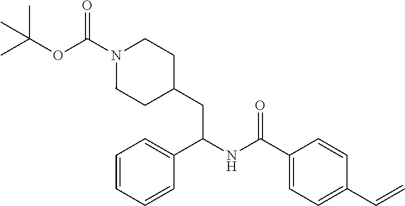 [SMILES: C=Cc1ccc(C(=O)NC(CC2CCN(C(=O)OC(C)(C)C)CC2)c2ccccc2)cc1]

### Example 120: ethyl 4-(2-(4-fluorobenzamido)-2-phenylethyl)piperidine-1-carboxylate

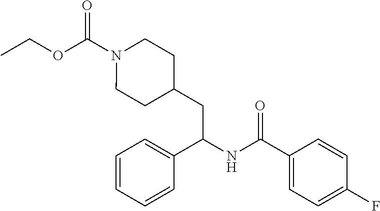 [SMILES: CCOC(=O)N1CCC(CC(NC(=O)c2ccc(F)cc2)c2ccccc2)CC1]

### Example 121: phenyl 4-(2-(4-fluorobenzamido)-2-phenylethyl)piperidine-1-carboxylate

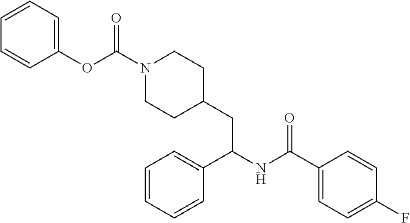 [SMILES: O=C(NC(CC1CCN(C(=O)Oc2ccccc2)CC1)c1ccccc1)c1ccc(F)cc1]

### Example 122: tert-butyl 4-(2-(6-(diethylamino)pyridin-3-yl)-2-(4-fluorobenzamido)ethyl)piperidine-1-carboxylate

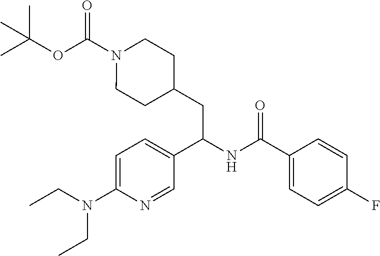 [SMILES: CCN(CC)c1ccc(C(CC2CCN(C(=O)OC(C)(C)C)CC2)NC(=O)c2ccc(F)cc2)cn1]

### Example 123: (S)-tert-butyl 4-(2-(6-methoxynicotinamido)-2-phenylethyl)piperidine-1-carboxylate

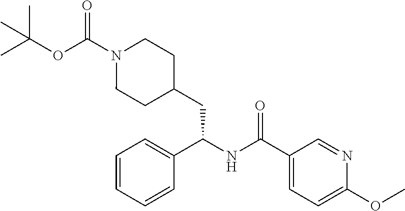 [SMILES: COc1ccc(C(=O)N[C@@H](CC2CCN(C(=O)OC(C)(C)C)CC2)c2ccccc2)cn1]

### Example 124: tert-butyl 4-(2-(5-isopropylthiophene-2-carboxamido)-2-phenylethyl)piperidine-1-carboxylate

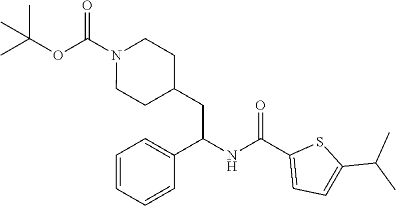 [SMILES: CC(C)c1ccc(C(=O)NC(CC2CCN(C(=O)OC(C)(C)C)CC2)c2ccccc2)s1]

### Example 125: isobutyl 4-(2-(4-chlorobenzamido)-2-phenylethyl)piperidine-1-carboxylate

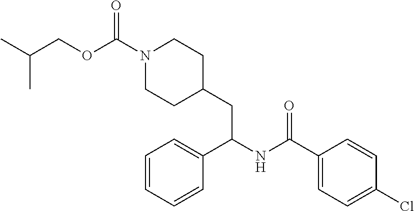 [SMILES: CC(C)COC(=O)N1CCC(CC(NC(=O)c2ccc(Cl)cc2)c2ccccc2)CC1]

### Example 126: tert-butyl 4-(2-(4-bromobenzamido)-2-cyclopentylethyl)piperidine-1-carboxylate

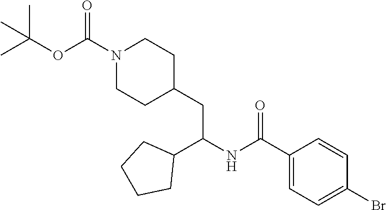 [SMILES: CC(C)(C)OC(=O)N1CCC(CC(NC(=O)c2ccc(Br)cc2)C2CCCC2)CC1]

### Example 127: tert-butyl 4-(2-(5-chlorothiophene-2-carboxamido)-2-(5-(ethylamino)-1,3,4-oxadiazol-2-yl)ethyl)piperidine-1-carboxylate

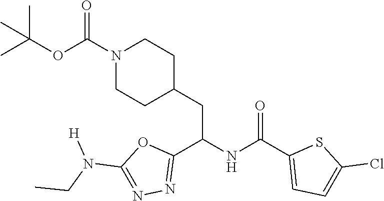

### Example 128: tert-butyl 4-(2-(5-chlorothiophene-3-carboxamido)-2-phenylethyl)piperidine-1-carboxylate

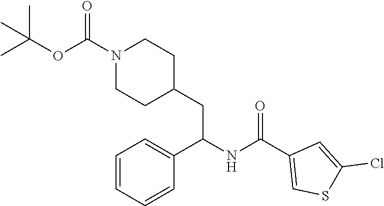 [SMILES: CC(C)(C)OC(=O)N1CCC(CC(NC(=O)c2csc(Cl)c2)c2ccccc2)CC1]

### Example 129: (S)-tert-butyl 4-(2-(5-chlorothiophene-2-carboxamido)-4-morpholinobutyl)piperidine-1-carboxylate

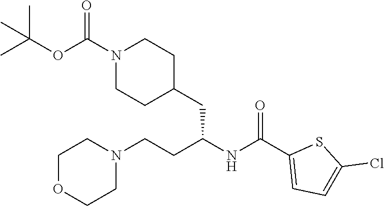 [SMILES: CC(C)(C)OC(=O)N1CCC(C[C@@H](CCN2CCOCC2)NC(=O)c2ccc(Cl)s2)CC1]

### Example 130: tert-butyl 4-(2-(4-methylbenzamido)-2-phenylethyl)piperidine-1-carboxylate

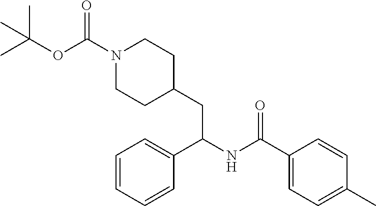 [SMILES: Cc1ccc(C(=O)NC(CC2CCN(C(=O)OC(C)(C)C)CC2)c2ccccc2)cc1]

### Example 131: tert-butyl 4-(2-phenyl-2-(thiophene-2-carboxamido)ethyl)piperidine-1-carboxylate

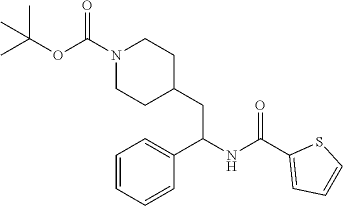 [SMILES: CC(C)(C)OC(=O)N1CCC(CC(NC(=O)c2cccs2)c2ccccc2)CC1]

### Example 132: tert-butyl 4-(2-(6-(cyclohexyloxy)pyridin-3-yl)-2-(4-fluorobenzamido)ethyl)piperidine-1-carboxylate

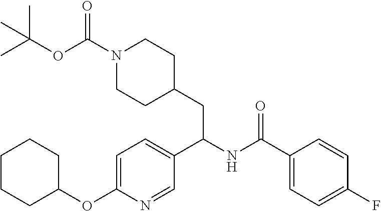 [SMILES: CC(C)(C)OC(=O)N1CCC(CC(NC(=O)c2ccc(F)cc2)c2ccc(OC3CCCCC3)nc2)CC1]

### Example 133: tert-butyl 4-(2-(4-cyclopropyl-3-fluorobenzamido)-2-phenylethyl)piperidine-1-carboxylate

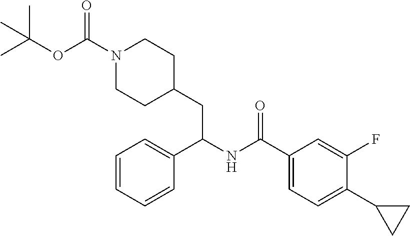 [SMILES: CC(C)(C)OC(=O)N1CCC(CC(NC(=O)c2ccc(C3CC3)c(F)c2)c2ccccc2)CC1]

### Example 134: methyl 4-(2-(4-chlorobenzamido)-2-phenylethyl)piperidine-1-carboxylate

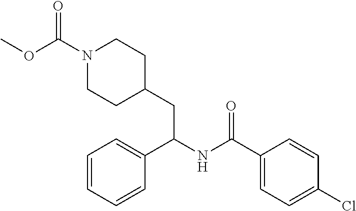 [SMILES: COC(=O)N1CCC(CC(NC(=O)c2ccc(Cl)cc2)c2ccccc2)CC1]

### Example 135: tert-butyl 4-(2-(4-chlorobenzamido)-2-cyclohexylethyl)piperidine-1-carboxylate

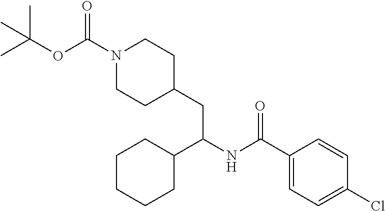 [SMILES: CC(C)(C)OC(=O)N1CCC(CC(NC(=O)c2ccc(Cl)cc2)C2CCCCC2)CC1]

### Example 136: N-(2-(1-(2,2-difluoro-2-(pyridin-2-yl)acetyl)piperidin-4-yl)-1-phenylethyl)-5-methylthiophene-2-carboxamide

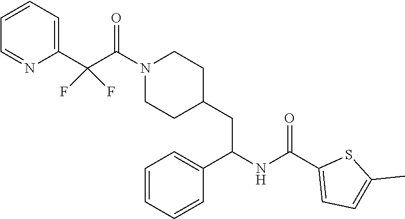 [SMILES: Cc1ccc(C(=O)NC(CC2CCN(C(=O)C(F)(F)c3ccccn3)CC2)c2ccccc2)s1]

### Example 137: 5-methyl-N-(1-phenyl-2-(1-phenylpiperidin-4-yl)ethyl)thiophene-2-carboxamide

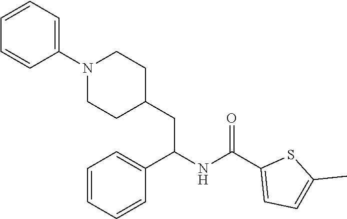 [SMILES: Cc1ccc(C(=O)NC(CC2CCN(c3ccccc3)CC2)c2ccccc2)s1]

### Example 138: tert-butyl 4-(2-benzamido-2-phenylethyl)piperidine-1-carboxylate

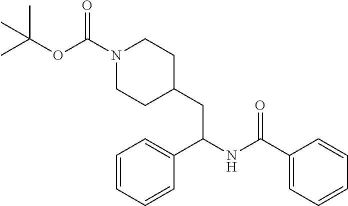 [SMILES: CC(C)(C)OC(=O)N1CCC(CC(NC(=O)c2ccccc2)c2ccccc2)CC1]

### Example 139: tert-butyl 4-(2-(3-fluoro-4-methylbenzamido)-2-phenylethyl)piperidine-1-carboxylate

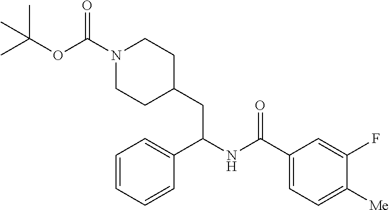 [SMILES: Cc1ccc(C(=O)NC(CC2CCN(C(=O)OC(C)(C)C)CC2)c2ccccc2)cc1F]

### Example 140: tert-butyl 4-(2-(5-(benzyl(methyl)amino)-1,3,4-oxadiazol-2-yl)-2-(5-chlorothiophene-2-carboxamido)ethyl)piperidine-1-carboxylate

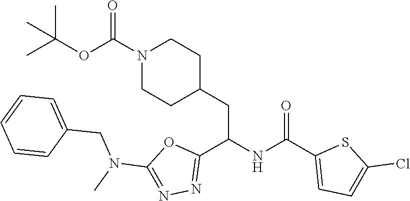 [SMILES: CN(Cc1ccccc1)c1nnc(C(CC2CCN(C(=O)OC(C)(C)C)CC2)NC(=O)c2ccc(Cl)s2)o1]

### Example 141: tert-butyl 4-(2-(4-cyanobenzamido)-2-phenylethyl)piperidine-1-carboxylate

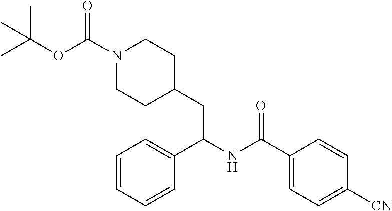 [SMILES: CC(C)(C)OC(=O)N1CCC(CC(NC(=O)c2ccc(C#N)cc2)c2ccccc2)CC1]

### Example 142: tert-butyl 4-(3-(benzylamino)-2-(4-fluorobenzamido)propyl)piperidine-1-carboxylate

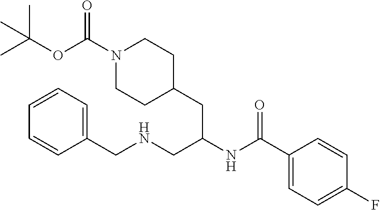 [SMILES: CC(C)(C)OC(=O)N1CCC(CC(CNCc2ccccc2)NC(=O)c2ccc(F)cc2)CC1]

### Example 143: tert-butyl 4-(2-(4-methoxybenzamido)-2-phenylethyl)piperidine-1-carboxylate

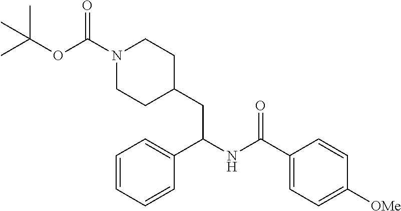 [SMILES: COc1ccc(C(=O)NC(CC2CCN(C(=O)OC(C)(C)C)CC2)c2ccccc2)cc1]

### Example 144: tert-butyl 4-(2-(4-fluorobenzamido)-2-(6-isopropoxypyridin-3-yl)ethyl)piperidine-1-carboxylate

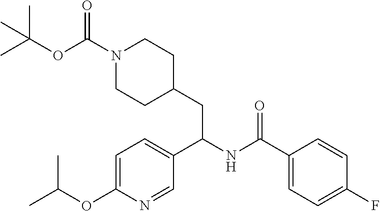 [SMILES: CC(C)Oc1ccc(C(CC2CCN(C(=O)OC(C)(C)C)CC2)NC(=O)c2ccc(F)cc2)cn1]

### Example 145: methyl 4-(2-(4-fluorobenzamido)-2-(6-methoxypyridin-3-yl)ethyl)piperidine-1-carboxylate

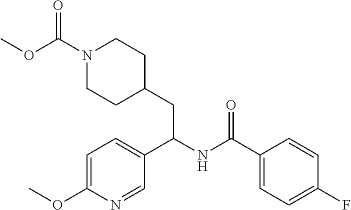 [SMILES: COC(=O)N1CCC(CC(NC(=O)c2ccc(F)cc2)c2ccc(OC)nc2)CC1]

### Example 146: tert-butyl 4-(2-(3-fluorobenzamido)-2-phenylethyl)piperidine-1-carboxylate

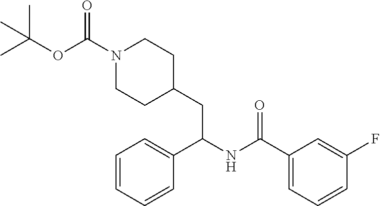 [SMILES: CC(C)(C)OC(=O)N1CCC(CC(NC(=O)c2cccc(F)c2)c2ccccc2)CC1]

### Example 147: tert-butyl 4-(2-(3,4-difluorobenzamido)-2-phenylethyl)piperidine-1-carboxylate

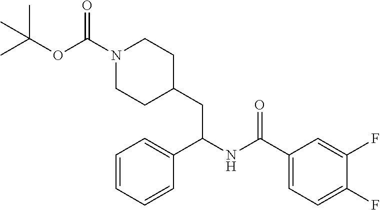 [SMILES: CC(C)(C)OC(=O)N1CCC(CC(NC(=O)c2ccc(F)c(F)c2)c2ccccc2)CC1]

### Example 148: (S)-tert-butyl 4-(2-((6-chlorobenzo[d]oxazol-2-yl)amino)-2-cyclopropylethyl)piperidine-1-carboxylate

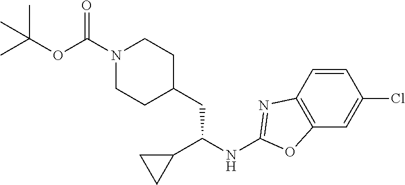 [SMILES: CC(C)(C)OC(=O)N1CCC(C[C@H](Nc2nc3ccc(Cl)cc3o2)C2CC2)CC1]

### Example 149: tert-butyl 4-(2-(4-(but-2-yn-1-yloxy)benzamido)-2-phenylethyl)piperidine-1-carboxylate

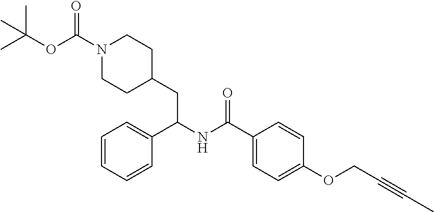 [SMILES: CC#CCOc1ccc(C(=O)NC(CC2CCN(C(=O)OC(C)(C)C)CC2)c2ccccc2)cc1]

### Example 150: cyclohexyl 4-(2-(4-chlorobenzamido)-2-phenylethyl)piperidine-1-carboxylate

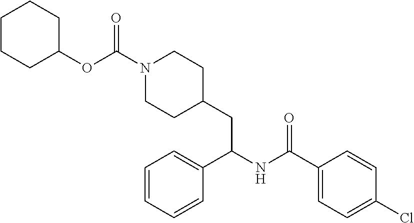 [SMILES: O=C(NC(CC1CCN(C(=O)OC2CCCCC2)CC1)c1ccccc1)c1ccc(Cl)cc1]

### Example 151: tert-butyl 4-(2-(4-chlorobenzamido)-2-cyclopentylethyl)piperidine-1-carboxylate

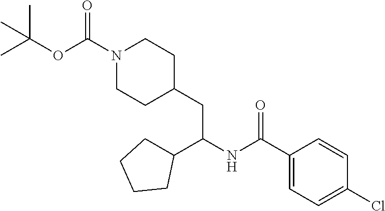 [SMILES: CC(C)(C)OC(=O)N1CCC(CC(NC(=O)c2ccc(Cl)cc2)C2CCCC2)CC1]

### Example 152: tert-butyl 4-(2-cyclohexyl-2-(4-fluorobenzamido)ethyl)piperidine-1-carboxylate

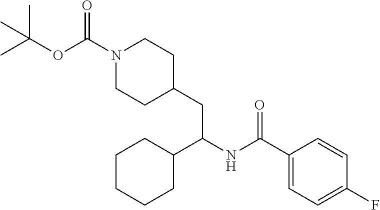 [SMILES: CC(C)(C)OC(=O)N1CCC(CC(NC(=O)c2ccc(F)cc2)C2CCCCC2)CC1]

### Example 153: tert-butyl 4-(2-(5-methylthiophene-3-carboxamido)-2-phenylethyl)piperidine-1-carboxylate

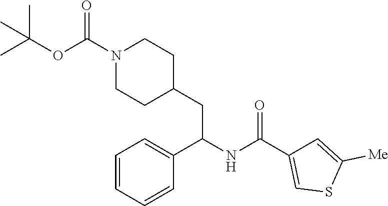 [SMILES: Cc1cc(C(=O)NC(CC2CCN(C(=O)OC(C)(C)C)CC2)c2ccccc2)cs1]

### Example 154: N-(2-(1-butyrylpiperidin-4-yl)-1-phenylethyl)-4-chlorobenzamide

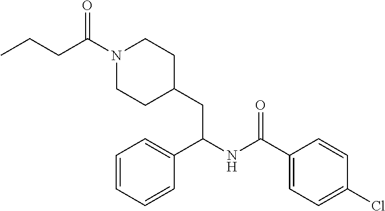 [SMILES: CCCC(=O)N1CCC(CC(NC(=O)c2ccc(Cl)cc2)c2ccccc2)CC1]

### Example 155: (S)-tert-butyl 4-(2-(5-chlorothiophene-2-carboxamido)pent-4-en-1-yl)piperidine-1-carboxylate

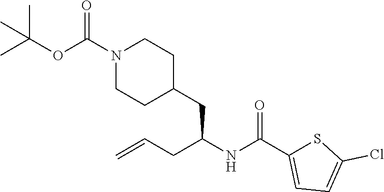 [SMILES: C=CC[C@@H](CC1CCN(C(=O)OC(C)(C)C)CC1)NC(=O)c1ccc(Cl)s1]

### Example 156: N-(2-(1-benzoylpiperidin-4-yl)-1-phenylethyl)-5-methylthiophene-2-carboxamide

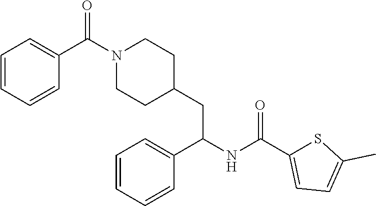 [SMILES: Cc1ccc(C(=O)NC(CC2CCN(C(=O)c3ccccc3)CC2)c2ccccc2)s1]

### Example 157: tert-butyl 4-(2-(6-methoxynicotinamido)-2-phenylethyl)piperidine-1-carboxylate

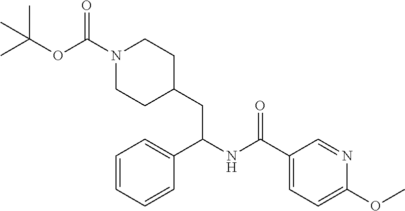 [SMILES: COc1ccc(C(=O)NC(CC2CCN(C(=O)OC(C)(C)C)CC2)c2ccccc2)cn1]

### Example 158: ethyl 4-(2-cyclopropyl-2-(6-methoxynicotinamido)ethyl)piperidine-1-carboxylate

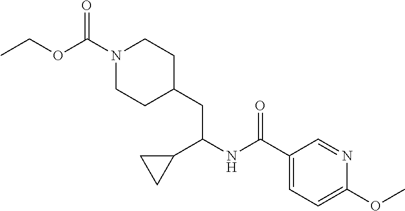 [SMILES: CCOC(=O)N1CCC(CC(NC(=O)c2ccc(OC)nc2)C2CC2)CC1]

### Example 159: tert-butyl 4-(2-phenyl-2-(thiophene-3-carboxamido)ethyl)piperidine-1-carboxylate

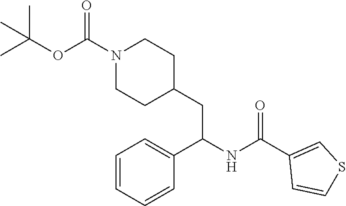 [SMILES: CC(C)(C)OC(=O)N1CCC(CC(NC(=O)c2ccsc2)c2ccccc2)CC1]

### Example 160: tert-butyl 4-(2-(6-(benzyloxy)pyridin-3-yl)-2-(4-fluorobenzamido)ethyl)piperidine-1-carboxylate

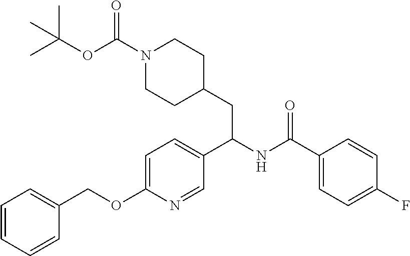 [SMILES: CC(C)(C)OC(=O)N1CCC(CC(NC(=O)c2ccc(F)cc2)c2ccc(OCc3ccccc3)nc2)CC1]

### Example 161: tert-butyl 4-(2-(4-chloro-3-methoxybenzamido)-2-phenylethyl)piperidine-1-carboxylate

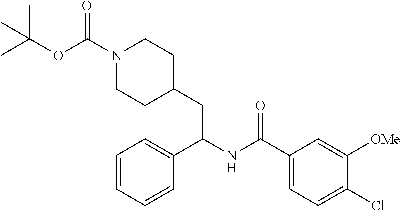 [SMILES: COc1cc(C(=O)NC(CC2CCN(C(=O)OC(C)(C)C)CC2)c2ccccc2)ccc1Cl]

### Example 162: tert-butyl 4-(2-(4-cyano-3-fluorobenzamido)-2-phenylethyl)piperidine-1-carboxylate

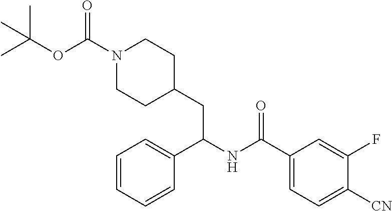 [SMILES: CC(C)(C)OC(=O)N1CCC(CC(NC(=O)c2ccc(C#N)c(F)c2)c2ccccc2)CC1]

### Example 163:4-chloro-N-(2-(1-(3-methylbutanoyl)piperidin-4-yl)-1-phenylethyl)benzamide

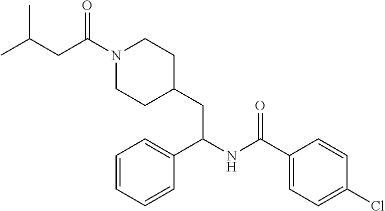 [SMILES: CC(C)CC(=O)N1CCC(CC(NC(=O)c2ccc(Cl)cc2)c2ccccc2)CC1]

### Example 164: 4-chloro-N-(2-(1-(3,3-dimethylbutanoyl)piperidin-4-yl)-1-phenylethyl)benzamide

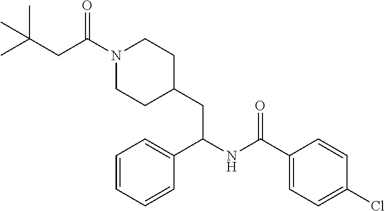 [SMILES: CC(C)(C)CC(=O)N1CCC(CC(NC(=O)c2ccc(Cl)cc2)c2ccccc2)CC1]

### Example 165: N-(tert-butyl)-4-(2-(4-chlorobenzamido)-2-phenylethyl)piperidine-1-carboxamide

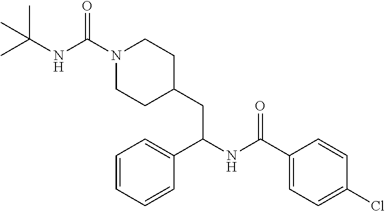 [SMILES: CC(C)(C)NC(=O)N1CCC(CC(NC(=O)c2ccc(Cl)cc2)c2ccccc2)CC1]

### Example 166: 4-chloro-N-(2-(1-(isobutylsulfonyl)piperidin-4-yl)-1-phenylethyl)benzamide

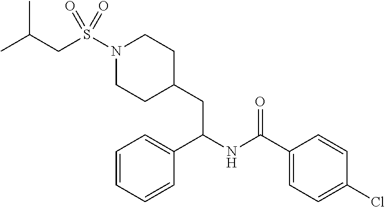 [SMILES: CC(C)CS(=O)(=O)N1CCC(CC(NC(=O)c2ccc(Cl)cc2)c2ccccc2)CC1]

### Example 167: tert-butyl 4-(2-(4-(but-2-yn-1-yloxy)-3-fluorobenzamido)-2-phenylethyl)piperidine-1-carboxylate

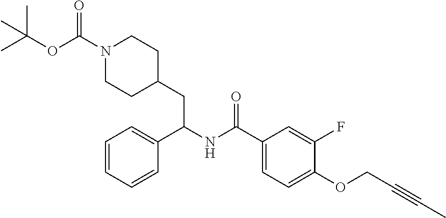 [SMILES: CC#CCOc1ccc(C(=O)NC(CC2CCN(C(=O)OC(C)(C)C)CC2)c2ccccc2)cc1F]

### Example 168: tert-butyl 4-(2-(4-chlorothiophene-2-carboxamido)-2-phenylethyl)piperidine-1-carboxylate

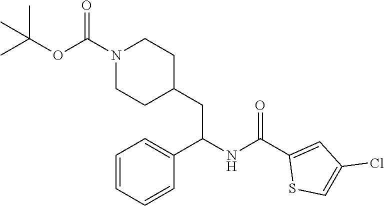 [SMILES: CC(C)(C)OC(=O)N1CCC(CC(NC(=O)c2cc(Cl)cs2)c2ccccc2)CC1]

### Example 169: tert-butyl 4-(2-(4-fluorobenzamido)-2-(5-(pentan-3-yl)-1,2,4-oxadiazol-3-yl)ethyl)piperidine-1-carboxylate

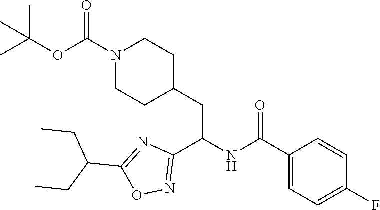 [SMILES: CCC(CC)c1nc(C(CC2CCN(C(=O)OC(C)(C)C)CC2)NC(=O)c2ccc(F)cc2)no1]

### Example 170: (S)-tert-butyl 4-(2-cyclopropyl-2-((5-phenyloxazol-2-yl)amino)ethyl)piperidine-1-carboxylate

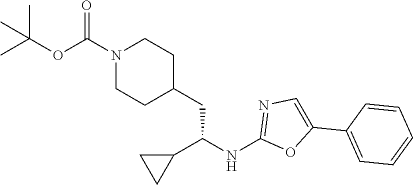 [SMILES: CC(C)(C)OC(=O)N1CCC(C[C@H](Nc2ncc(-c3ccccc3)o2)C2CC2)CC1]

### Example 171: tert-butyl 4-(2-(5-chlorothiophene-2-carboxamido)-2-(5-(propylamino)-1,3,4-oxadiazol-2-yl)ethyl)piperidine-1-carboxylate

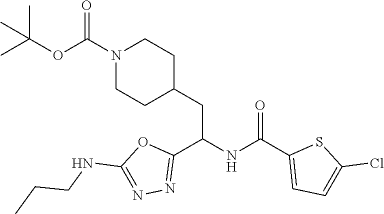 [SMILES: CCCNc1nnc(C(CC2CCN(C(=O)OC(C)(C)C)CC2)NC(=O)c2ccc(Cl)s2)o1]

### Example 172: 5-methyl-N-(1-phenyl-2-(1-(3,3,3-trifluoropropanoyl)piperidin-4-yl)ethyl)thiophene-2-carboxamide

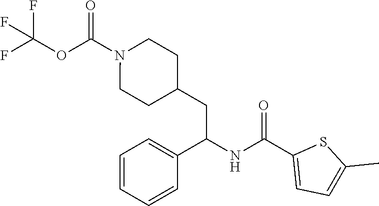 [SMILES: Cc1ccc(C(=O)NC(CC2CCN(C(=O)OC(F)(F)F)CC2)c2ccccc2)s1]

### Example 173: methyl 4-(2-(4-fluorobenzamido)-2-phenylethyl)piperidine-1-carboxylate

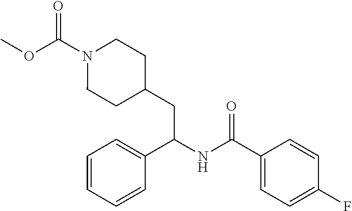 [SMILES: COC(=O)N1CCC(CC(NC(=O)c2ccc(F)cc2)c2ccccc2)CC1]

### Example 174: tert-butyl 4-(2-(4-fluorobenzamido)-2-(6-methylpyridin-3-yl)ethyl)piperidine-1-carboxylate

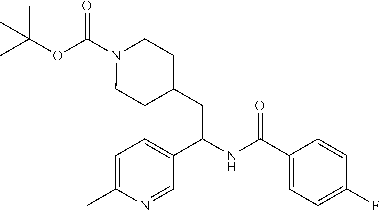 [SMILES: Cc1ccc(C(CC2CCN(C(=O)OC(C)(C)C)CC2)NC(=O)c2ccc(F)cc2)cn1]

### Example 175: tert-butyl 4-(2-(benzo[d][1,3]dioxole-5-carboxamido)-2-phenylethyl)piperidine-1-carboxylate

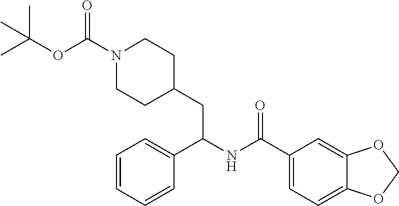 [SMILES: CC(C)(C)OC(=O)N1CCC(CC(NC(=O)c2ccc3c(c2)OCO3)c2ccccc2)CC1]

### Example 176: tert-butyl 4-(2-(1-benzyl-6-oxo-1,6-dihydropyridin-3-yl)-2-(4-fluorobenzamido)ethyl)piperidine-1-carboxylate

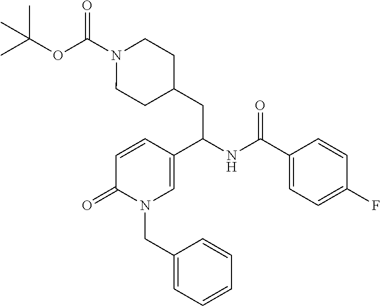 [SMILES: CC(C)(C)OC(=O)N1CCC(CC(NC(=O)c2ccc(F)cc2)c2ccc(=O)n(Cc3ccccc3)c2)CC1]

### Example 177: (R)-ethyl 4-(2-(5-chlorothiophene-2-carboxamido)-2-cyclopropylethyl)piperdine-1-carboxylate

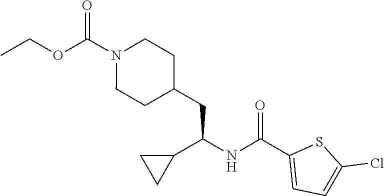 [SMILES: CCOC(=O)N1CCC(C[C@@H](NC(=O)c2ccc(Cl)s2)C2CC2)CC1]

### Example 178: tert-butyl 4-(2-(4-(methylthio)benzamido)-2-phenylethyl)piperidine-1-carboxylate

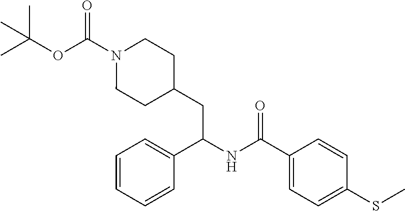 [SMILES: CSc1ccc(C(=O)NC(CC2CCN(C(=O)OC(C)(C)C)CC2)c2ccccc2)cc1]

### Example 179: tert-butyl 4-(2-(4-methylthiophene-2-carboxamido)-2-phenylethyl)piperidine-1-carboxylate

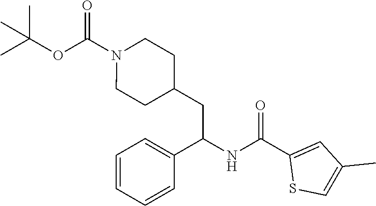 [SMILES: Cc1csc(C(=O)NC(CC2CCN(C(=O)OC(C)(C)C)CC2)c2ccccc2)c1]

### Example 180: tert-butyl 4-(2-phenyl-2-(5-vinylthiophene-2-carboxamido)ethyl)piperidine-1-carboxylate

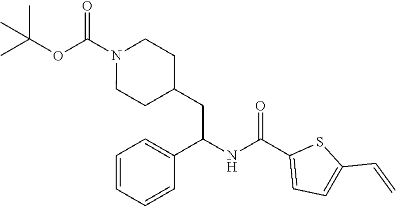 [SMILES: C=Cc1ccc(C(=O)NC(CC2CCN(C(=O)OC(C)(C)C)CC2)c2ccccc2)s1]

### Example 181: tert-butyl 4-(2-(1H-indole-7-carboxamido)-2-phenylethyl)piperidine-1-carboxylate

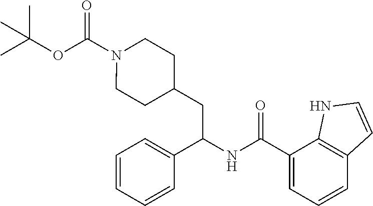 [SMILES: CC(C)(C)OC(=O)N1CCC(CC(NC(=O)c2cccc3cc[nH]c23)c2ccccc2)CC1]

### Example 182: tert-butyl 4-(2-(benzo[b]thiophene-2-carboxamido)-2-phenylethyl)piperidine-1-carboxylate

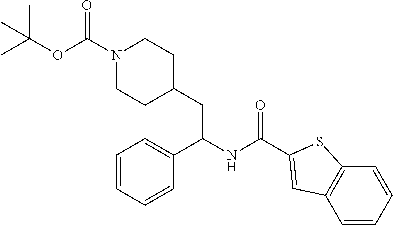 [SMILES: CC(C)(C)OC(=O)N1CCC(CC(NC(=O)c2cc3ccccc3s2)c2ccccc2)CC1]

### Example 183: (R)-tert-butyl 4-(2-(4-fluorobenzamido)-2-(6-methoxypyridin-3-yl)ethyl)piperidine-1-carboxylate

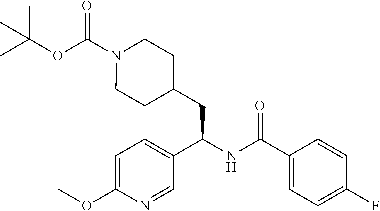 [SMILES: COc1ccc([C@@H](CC2CCN(C(=O)OC(C)(C)C)CC2)NC(=O)c2ccc(F)cc2)cn1]

### Example 184: 4-(2-(4-chlorobenzamido)-2-phenylethyl)-N-isopropylpiperidine-1-carboxamide

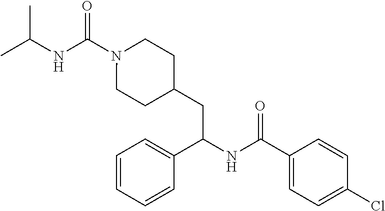 [SMILES: CC(C)NC(=O)N1CCC(CC(NC(=O)c2ccc(Cl)cc2)c2ccccc2)CC1]

### Example 185: oxetan-3-yl 4-(2-(4-chlorobenzamido)-2-phenylethyl)piperidine-1-carboxylate

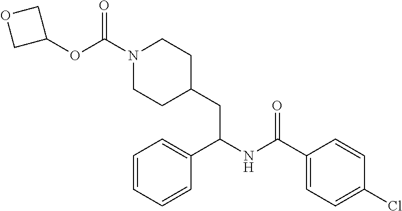 [SMILES: O=C(NC(CC1CCN(C(=O)OC2COC2)CC1)c1ccccc1)c1ccc(Cl)cc1]

### Example 186: tetrahydro-2H-pyran-4-yl 4-(2-(4-chlorobenzamido)-2-phenylethyl)piperidine-1-carboxylate

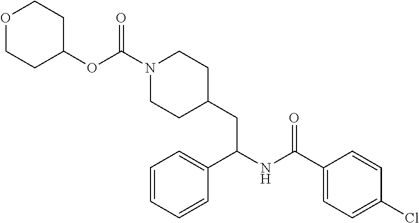 [SMILES: O=C(NC(CC1CCN(C(=O)OC2CCOCC2)CC1)c1ccccc1)c1ccc(Cl)cc1]

### Example 187: neopentyl 4-(2-(4-chlorobenzamido)-2-phenylethyl)piperidine-1-carboxylate

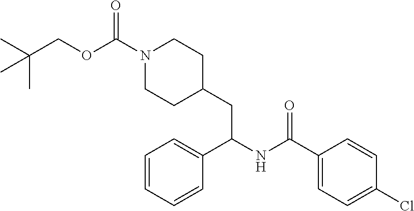 [SMILES: CC(C)(C)COC(=O)N1CCC(CC(NC(=O)c2ccc(Cl)cc2)c2ccccc2)CC1]

### Example 188: (R)-isopropyl 4-(2-(5-chlorothiophene-2-carboxamido)-2-cyclopropylethyl)piperidine-1-carboxylate

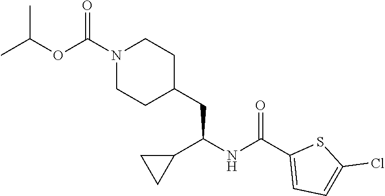 [SMILES: CC(C)OC(=O)N1CCC(C[C@@H](NC(=O)c2ccc(Cl)s2)C2CC2)CC1]

### Example 189: tert-butyl 4-(2-(6-cyanonicotinamido)-2-phenylethyl)piperidine-1-carboxylate

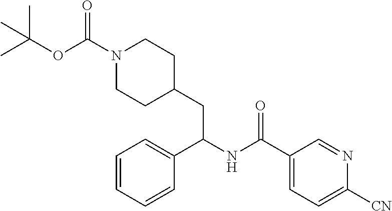 [SMILES: CC(C)(C)OC(=O)N1CCC(CC(NC(=O)c2ccc(C#N)nc2)c2ccccc2)CC1]

### Example 190: tert-butyl 4-(2-(cyclohexanecarboxamido)-2-phenylethyl)piperidine-1-carboxylate

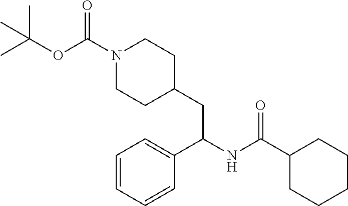 [SMILES: CC(C)(C)OC(=O)N1CCC(CC(NC(=O)C2CCCCC2)c2ccccc2)CC1]

### Example 191: tert-butyl 4-(2-(2-methylthiazole-5-carboxamido)-2-phenylethyl)piperidine-1-carboxylate

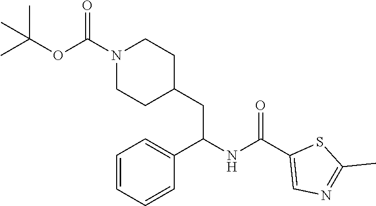 [SMILES: Cc1ncc(C(=O)NC(CC2CCN(C(=O)OC(C)(C)C)CC2)c2ccccc2)s1]

### Example 192: tert-butyl 4-(2-phenyl-2-(4-propoxybenzamido)ethyl)piperidine-1-carboxylate

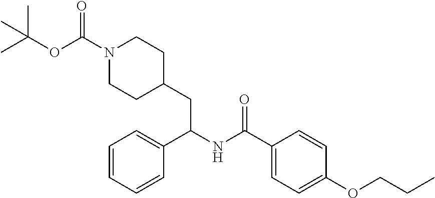 [SMILES: CCCOc1ccc(C(=O)NC(CC2CCN(C(=O)OC(C)(C)C)CC2)c2ccccc2)cc1]

### Example 193: 4-chloro-N-(2-(1-(cyclopentylsulfonyl)piperidin-4-yl)-1-phenylethyl)benzamide

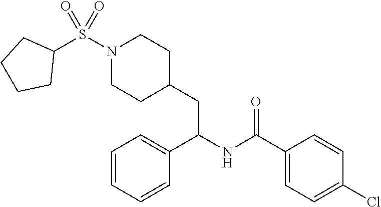 [SMILES: O=C(NC(CC1CCN(S(=O)(=O)C2CCCC2)CC1)c1ccccc1)c1ccc(Cl)cc1]

### Example 194: tert-butyl 4-(2-(2-hydroxybenzamido)-2-phenylethyl)piperidine-1-carboxylate

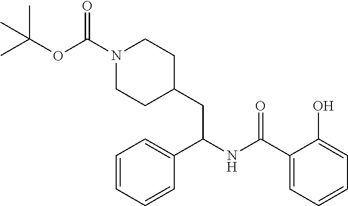 [SMILES: CC(C)(C)OC(=O)N1CCC(CC(NC(=O)c2ccccc2O)c2ccccc2)CC1]

### Example 195: tert-butyl 4-(2-(4-hydroxybenzamido)-2-phenylethyl)piperidine-1-carboxylate

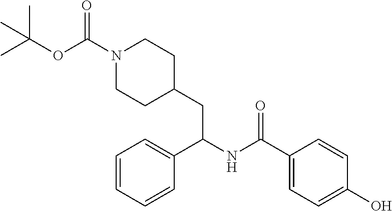 [SMILES: CC(C)(C)OC(=O)N1CCC(CC(NC(=O)c2ccc(O)cc2)c2ccccc2)CC1]

### Example 196: tert-butyl 4-(2-(3-fluoro-4-hydroxybenzamido)-2-phenylethyl)piperidine-1-carboxylate

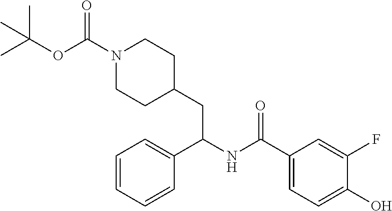 [SMILES: CC(C)(C)OC(=O)N1CCC(CC(NC(=O)c2ccc(O)c(F)c2)c2ccccc2)CC1]

### Example 197:4-chloro-N-(1-phenyl-2-(1-(piperidin-1-ylsulfonyl)piperidin-4-yl)ethyl)benzamide

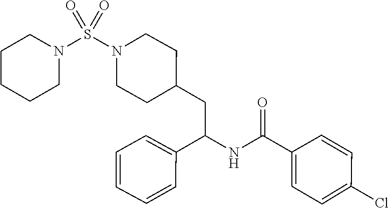 [SMILES: O=C(NC(CC1CCN(S(=O)(=O)N2CCCCC2)CC1)c1ccccc1)c1ccc(Cl)cc1]

### Example 198: tert-butyl 4-(2-(3-cyclopentyl-1,2,4-oxadiazol-5-yl)-2-(4-fluorobenzamido)ethyl)piperidine-1-carboxylate

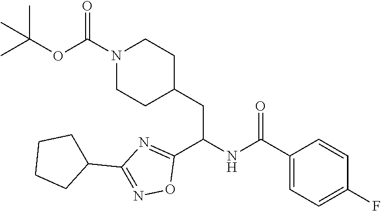 [SMILES: CC(C)(C)OC(=O)N1CCC(CC(NC(=O)c2ccc(F)cc2)c2nc(C3CCCC3)no2)CC1]

### Example 199: tert-butyl 4-(2-(3-chlorobenzamido)-2-phenylethyl)piperidine-1-carboxylate

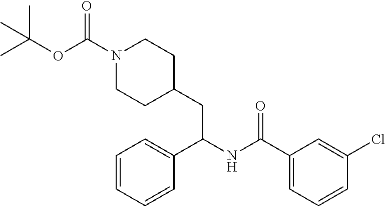 [SMILES: CC(C)(C)OC(=O)N1CCC(CC(NC(=O)c2cccc(Cl)c2)c2ccccc2)CC1]

### Example 200: tert-butyl 4-(2-(3-(4-fluorophenyl)ureido)-2-phenylethyl)piperidine-1-carboxylate

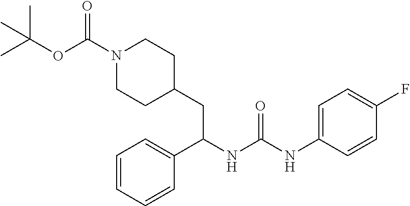 [SMILES: CC(C)(C)OC(=O)N1CCC(CC(NC(=O)Nc2ccc(F)cc2)c2ccccc2)CC1]

### Example 201: tert-butyl 4-(2-(bicyclo[2.2.2]octane-1-carboxamido)-2-phenylethyl)piperidine-1-carboxylate

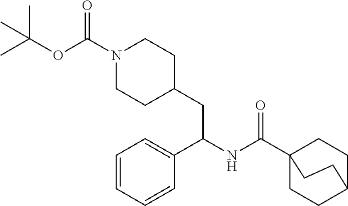 [SMILES: CC(C)(C)OC(=O)N1CCC(CC(NC(=O)C23CCC(CC2)CC3)c2ccccc2)CC1]

### Example 202: tert-butyl 4-(2-(4-fluorobenzamido)-2-(6-(2-methoxyethoxy)pyridin-3-yl)ethyl)piperidine-1-carboxylate

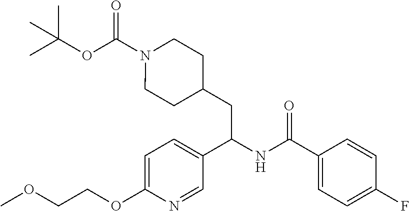 [SMILES: COCCOc1ccc(C(CC2CCN(C(=O)OC(C)(C)C)CC2)NC(=O)c2ccc(F)cc2)cn1]

### Example 203: tert-butyl 4-(2-(3-chloro-4-fluorobenzamido)-2-phenylethyl)piperidine-1-carboxylate

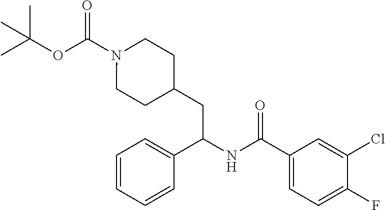 [SMILES: CC(C)(C)OC(=O)N1CCC(CC(NC(=O)c2ccc(F)c(Cl)c2)c2ccccc2)CC1]

### Example 204: tert-butyl 4-(2-(5-methylfuran-2-carboxamido)-2-phenylethyl)piperidine-1-carboxylate

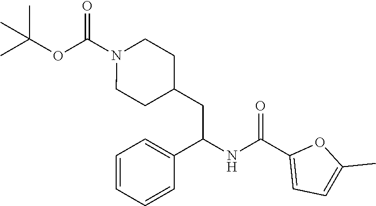 [SMILES: Cc1ccc(C(=O)NC(CC2CCN(C(=O)OC(C)(C)C)CC2)c2ccccc2)o1]

### Example 205: tert-butyl 4-(2-(3,4-dichlorobenzamido)-2-phenylethyl)piperidine-1-carboxylate

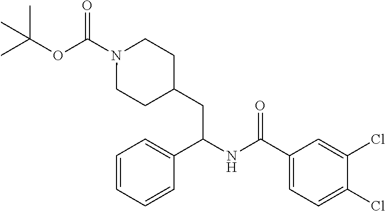 [SMILES: CC(C)(C)OC(=O)N1CCC(CC(NC(=O)c2ccc(Cl)c(Cl)c2)c2ccccc2)CC1]

### Example 206: (R)-methyl 4-(2-(5-chlorothiophene-2-carboxamido)-2-cyclopropylethyl)piperidine-1-carboxylate

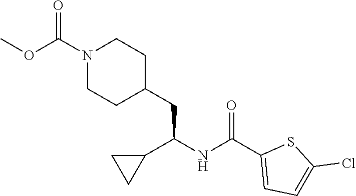 [SMILES: COC(=O)N1CCC(C[C@@H](NC(=O)c2ccc(Cl)s2)C2CC2)CC1]

### Example 207: tert-butyl 4-(2-(4-fluorobenzamido)-2-(pyridin-3-yl)ethyl)piperidine-1-carboxylate

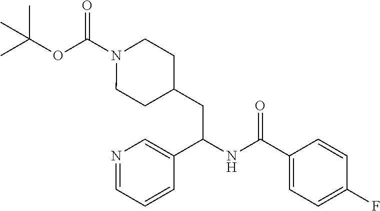 [SMILES: CC(C)(C)OC(=O)N1CCC(CC(NC(=O)c2ccc(F)cc2)c2cccnc2)CC1]

### Example 208: tert-butyl 4-(2-(4-fluorobenzamido)-2-(pyridin-4-yl)ethyl)piperidine-1-carboxylate

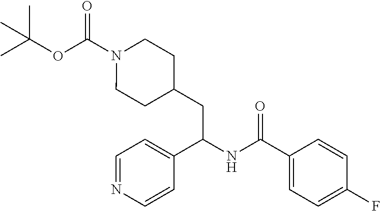 [SMILES: CC(C)(C)OC(=O)N1CCC(CC(NC(=O)c2ccc(F)cc2)c2ccncc2)CC1]

### Example 209: tert-butyl 4-(2-(cycloheptanecarboxamido)-2-phenylethyl)piperidine-1-carboxylate

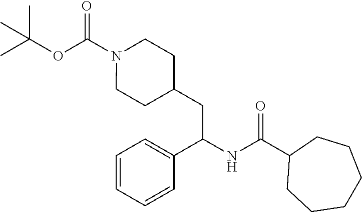 [SMILES: CC(C)(C)OC(=O)N1CCC(CC(NC(=O)C2CCCCCC2)c2ccccc2)CC1]

### Example 210: tert-butyl 4-(2-(3,3-dimethylbutanamido)-2-phenylethyl)piperidine-1-carboxylate

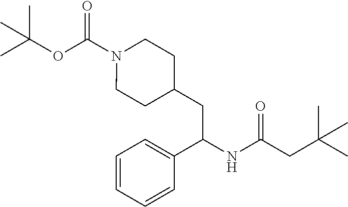 [SMILES: CC(C)(C)CC(=O)NC(CC1CCN(C(=O)OC(C)(C)C)CC1)c1ccccc1]

### Example 211: tert-butyl 4-(2-(2,5-difluorobenzamido)-2-phenylethyl)piperidine-1-carboxylate

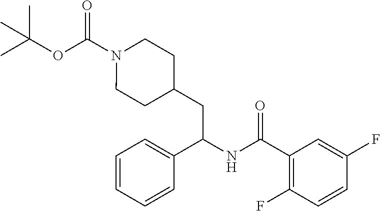 [SMILES: CC(C)(C)OC(=O)N1CCC(CC(NC(=O)c2cc(F)ccc2F)c2ccccc2)CC1]

### Example 212: tert-butyl 4-(2-(4-isopropylbenzamido)-2-phenylethyl)piperidine-1-carboxylate

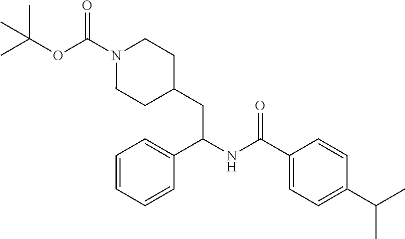 [SMILES: CC(C)c1ccc(C(=O)NC(CC2CCN(C(=O)OC(C)(C)C)CC2)c2ccccc2)cc1]

### Example 213: tert-butyl 4-(2-(4-fluoro-3-methylbenzamido)-2-phenylethyl)piperidine-1-carboxylate

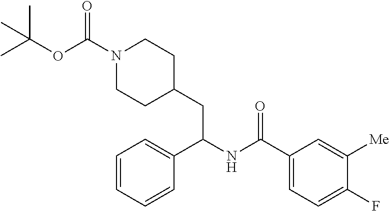 [SMILES: Cc1cc(C(=O)NC(CC2CCN(C(=O)OC(C)(C)C)CC2)c2ccccc2)ccc1F]

### Example 214: (R)-tert-butyl 4-(2-(5-chlorothiophene-2-carboxamido)-2-cyclopropylethyl)piperidine-1-carboxylate

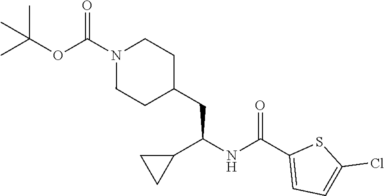 [SMILES: CC(C)(C)OC(=O)N1CCC(C[C@@H](NC(=O)c2ccc(Cl)s2)C2CC2)CC1]

### Example 215: 4-chloro-N-(1-phenyl-2-(1-(phenylsulfonyl)piperidin-4-yl)ethyl)benzamide

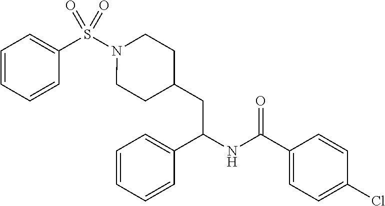 [SMILES: O=C(NC(CC1CCN(S(=O)(=O)c2ccccc2)CC1)c1ccccc1)c1ccc(Cl)cc1]

### Example 216: tert-butyl 4-(2-(3-methoxybenzamido)-2-phenylethyl)piperidine-1-carboxylate

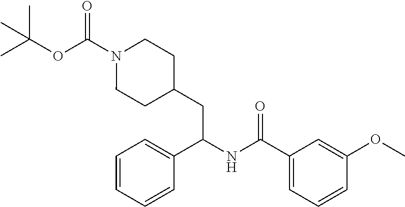 [SMILES: COc1cccc(C(=O)NC(CC2CCN(C(=O)OC(C)(C)C)CC2)c2ccccc2)c1]

### Example 217: tert-butyl 4-(2-(6-methylnicotinamido)-2-phenylethyl)piperidine-1-carboxylate

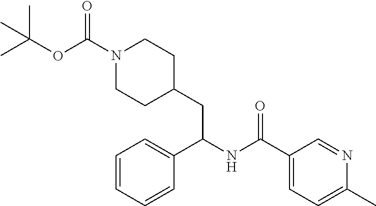 [SMILES: Cc1ccc(C(=O)NC(CC2CCN(C(=O)OC(C)(C)C)CC2)c2ccccc2)cn1]

### Example 218: tert-butyl 4-(2-(2-fluoro-4-methylbenzamido)-2-phenylethyl)piperidine-1-carboxylate

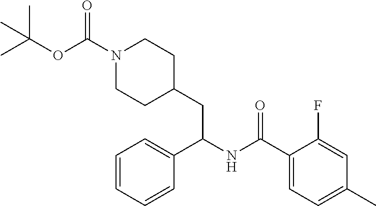 [SMILES: Cc1ccc(C(=O)NC(CC2CCN(C(=O)OC(C)(C)C)CC2)c2ccccc2)c(F)c1]

### Example 219: tert-butyl 4-(2-(2-fluorobenzamido)-2-phenylethyl)piperidine-1-carboxylate

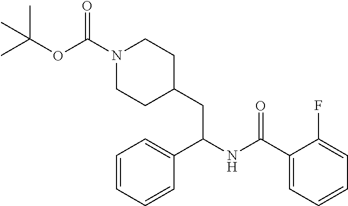 [SMILES: CC(C)(C)OC(=O)N1CCC(CC(NC(=O)c2ccccc2F)c2ccccc2)CC1]

### Example 220: tert-butyl 4-(2-phenyl-2-(1H-pyrrole-2-carboxamido)ethyl)piperidine-1-carboxylate

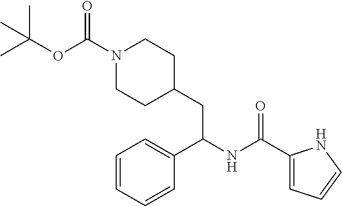 [SMILES: CC(C)(C)OC(=O)N1CCC(CC(NC(=O)c2ccc[nH]2)c2ccccc2)CC1]

### Example 221: tert-butyl 4-(2-(5-cyclopropyl-1,3,4-oxadiazol-2-yl)-2-(4-fluorobenzamido)ethyl)piperidine-1-carboxylate

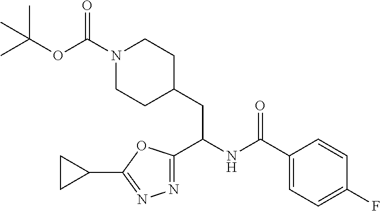 [SMILES: CC(C)(C)OC(=O)N1CCC(CC(NC(=O)c2ccc(F)cc2)c2nnc(C3CC3)o2)CC1]

### Example 222: N-(2-(1-acetylpiperidin-4-yl)-1-phenylethyl)-4-chlorobenzamide

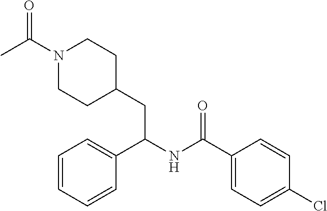 [SMILES: CC(=O)N1CCC(CC(NC(=O)c2ccc(Cl)cc2)c2ccccc2)CC1]

### Example 223: tert-butyl 4-(2-(4-fluorobenzamido)-2-(5-isopropyl-1,3,4-oxadiazol-2-yl)ethyl)piperidine-1-carboxylate

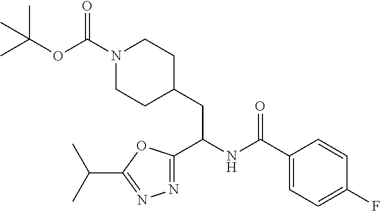 [SMILES: CC(C)c1nnc(C(CC2CCN(C(=O)OC(C)(C)C)CC2)NC(=O)c2ccc(F)cc2)o1]

### Example 224: tert-butyl 4-(2-(5-chlorothiophene-2-carboxamido)-2-(5-oxo-4,5-dihydro-1,3,4-oxadiazol-2-yl)ethyl)piperidine-1-carboxylate

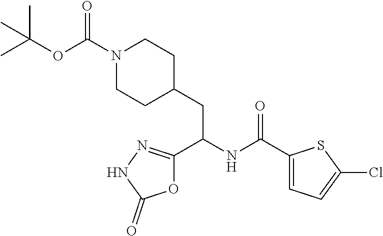 [SMILES: CC(C)(C)OC(=O)N1CCC(CC(NC(=O)c2ccc(Cl)s2)c2n[nH]c(=O)o2)CC1]

### Example 225: tert-butyl 4-(2-(4-fluorobenzamido)-3-(isobutylamino)propyl)piperidine-1-carboxylate

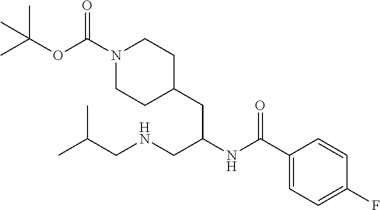 [SMILES: CC(C)CNCC(CC1CCN(C(=O)OC(C)(C)C)CC1)NC(=O)c1ccc(F)cc1]

### Example 226: tert-butyl 4-(2-(4-fluorobenzamido)-2-(pyridin-2-yl)ethyl)piperidine-1-carboxylate

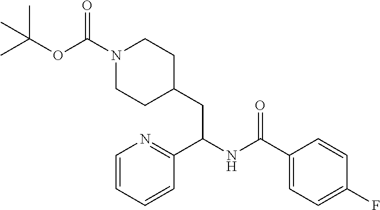 [SMILES: CC(C)(C)OC(=O)N1CCC(CC(NC(=O)c2ccc(F)cc2)c2ccccn2)CC1]

### Example 227: tert-butyl 4-(2-(5-isobutylthiophene-2-carboxamido)-2-phenylethyl)piperidine-1-carboxylate

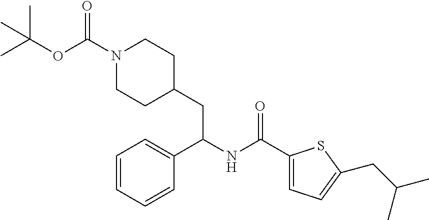 [SMILES: CC(C)Cc1ccc(C(=O)NC(CC2CCN(C(=O)OC(C)(C)C)CC2)c2ccccc2)s1]

### Example 228: tert-butyl 4-(2-(furan-2-carboxamido)-2-phenylethyl)piperidine-1-carboxylate

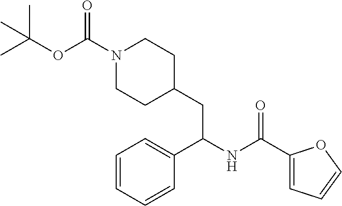 [SMILES: CC(C)(C)OC(=O)N1CCC(CC(NC(=O)c2ccco2)c2ccccc2)CC1]

### Example 229: (S)-tert-butyl 4-(2-((2-chloropyrimidin-4-yl)amino)-2-cyclopropylethyl)piperidine-1-carboxylate

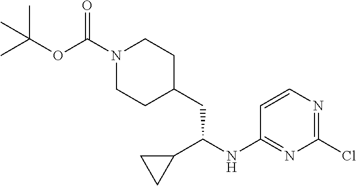 [SMILES: CC(C)(C)OC(=O)N1CCC(C[C@H](Nc2ccnc(Cl)n2)C2CC2)CC1]

### Example 230: 4-chloro-N-(1-phenyl-2-(1-(piperidine-1-carbonyl)piperidin-4-yl)ethyl)benzamide

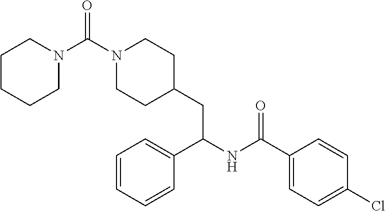 [SMILES: O=C(NC(CC1CCN(C(=O)N2CCCCC2)CC1)c1ccccc1)c1ccc(Cl)cc1]

### Example 231: (S)-tert-butyl 4-(2-(5-chlorothiophene-2-carboxamido)-4-(ethylamino)butyl)piperidine-1-carboxylate

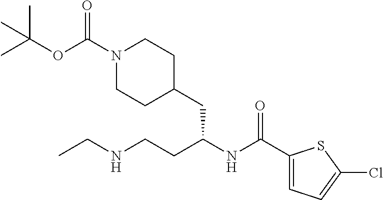 [SMILES: CCNCC[C@H](CC1CCN(C(=O)OC(C)(C)C)CC1)NC(=O)c1ccc(Cl)s1]

### Example 232: tert-butyl 4-(2-(cyclopentanecarboxamido)-2-phenylethyl)piperidine-1-carboxylate

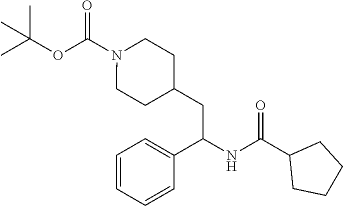 [SMILES: CC(C)(C)OC(=O)N1CCC(CC(NC(=O)C2CCCC2)c2ccccc2)CC1]

### Example 233: tert-butyl 4-(2-(4-methylcyclohexanecarboxamido)-2-phenylethyl)piperidine-1-carboxylate

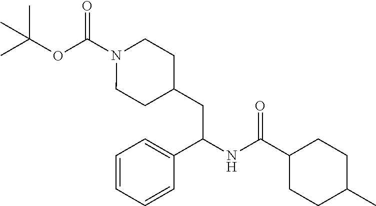 [SMILES: CC1CCC(C(=O)NC(CC2CCN(C(=O)OC(C)(C)C)CC2)c2ccccc2)CC1]

### Example 234: (R)-tert-butyl 4-(2-(4-chlorobenzamido)-2-phenylethyl)piperidine-1-carboxylate

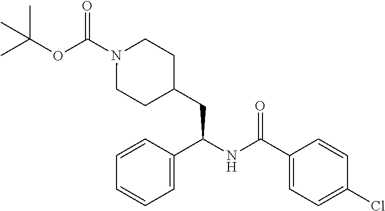 [SMILES: CC(C)(C)OC(=O)N1CCC(C[C@@H](NC(=O)c2ccc(Cl)cc2)c2ccccc2)CC1]

### Example 235: tert-butyl 4-(2-(4-fluorobenzamido)-3-((2-methoxyethyl)amino)propyl)piperidine-1-carboxylate

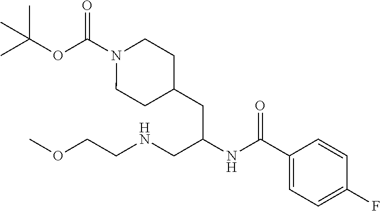 [SMILES: COCCNCC(CC1CCN(C(=O)OC(C)(C)C)CC1)NC(=O)c1ccc(F)cc1]

### Example 236: tert-butyl 4-(2-(nicotinamido)-2-phenylethyl)piperidine-1-carboxylate

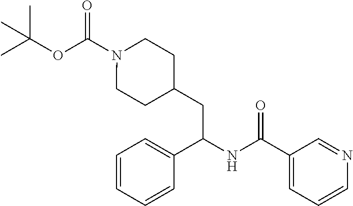 [SMILES: CC(C)(C)OC(=O)N1CCC(CC(NC(=O)c2cccnc2)c2ccccc2)CC1]

### Example 237: 4-fluoro-N-(2-(1-(3-methoxypropanoyl)piperidin-4-yl)-1-phenylethyl)benzamide

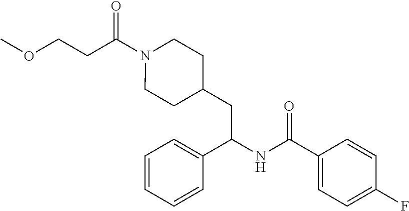 [SMILES: COCCC(=O)N1CCC(CC(NC(=O)c2ccc(F)cc2)c2ccccc2)CC1]

### Example 238: 4-fluoro-N-(2-(1-(3-methoxybutanoyl)piperidin-4-yl)-1-phenylethyl)benzamide

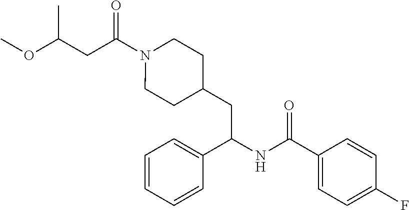 [SMILES: COC(C)CC(=O)N1CCC(CC(NC(=O)c2ccc(F)cc2)c2ccccc2)CC1]

### Example 239: tert-butyl 4-(2-(4-fluorobenzamido)-2-(5-methyloxazol-2-yl)ethyl)piperidine-1-carboxylate

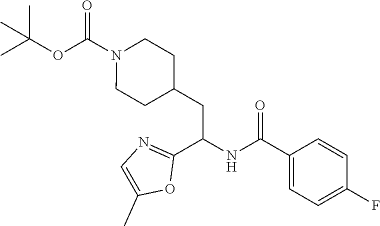 [SMILES: Cc1cnc(C(CC2CCN(C(=O)OC(C)(C)C)CC2)NC(=O)c2ccc(F)cc2)o1]

### Example 240: tert-butyl 4-(2-(1-methylcyclohexanecarboxamido)-2-phenylethyl)piperidine-1-carboxylate

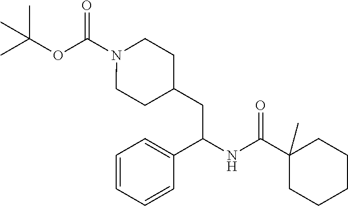 [SMILES: CC(C)(C)OC(=O)N1CCC(CC(NC(=O)C2(C)CCCCC2)c2ccccc2)CC1]

### Example 241: tert-butyl 4-(2-(4-isopropoxybenzamido)-2-phenylethyl)piperidine-1-carboxylate

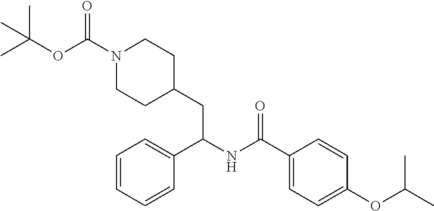 [SMILES: CC(C)Oc1ccc(C(=O)NC(CC2CCN(C(=O)OC(C)(C)C)CC2)c2ccccc2)cc1]

### Example 242: tert-butyl 4-(2-(4-isobutylbenzamido)-2-phenylethyl)piperidine-1-carboxylate

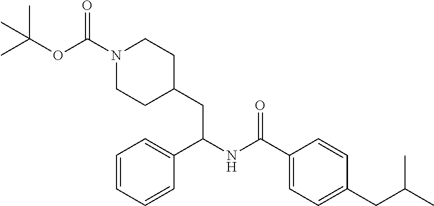 [SMILES: CC(C)Cc1ccc(C(=O)NC(CC2CCN(C(=O)OC(C)(C)C)CC2)c2ccccc2)cc1]

### Example 243: 4-chloro-N-(2-(1-(morpholine-4-carbonyl)piperidin-4-yl)-1-phenylethyl)benzamide

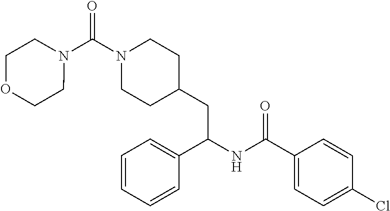 [SMILES: O=C(NC(CC1CCN(C(=O)N2CCOCC2)CC1)c1ccccc1)c1ccc(Cl)cc1]

### Example 244: 2-((5-(2-(1-(tert-butoxycarbonyl)piperidin-4-yl)-1-(5-chlorothiophene-2-carboxamido)ethyl)-1,3,4-oxadiazol-2-yl)(methyl)amino)acetic acid

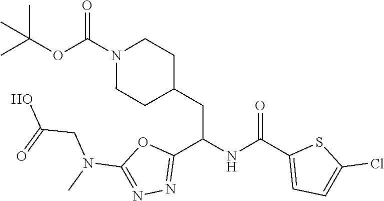 [SMILES: CN(CC(=O)O)c1nnc(C(CC2CCN(C(=O)OC(C)(C)C)CC2)NC(=O)c2ccc(Cl)s2)o1]

### Example 245: (S)-tert-butyl 4-(2-((5-chloropyridin-2-yl)amino)-2-cyclopropylethyl)piperidine-1-carboxylate

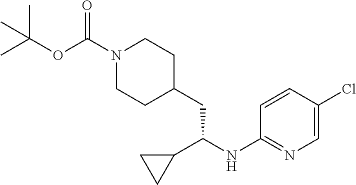 [SMILES: CC(C)(C)OC(=O)N1CCC(C[C@H](Nc2ccc(Cl)cn2)C2CC2)CC1]

### PBMC IDO1 Assay:

Data shown in Table 1. Compounds of the present invention were tested via high-throughput cellular assays utilizing detection of kynurenine via mass spectrometry and cytotoxicity as end-points. For the mass spectrometry and cytotoxicity assays, human peripheral blood mononuclear cells (PBMC) (PB003F; AIICells®, Alameda, Calif.) were stimulated with human interferon-γ (IFN-γ) (Sigma-Aldrich Corporation, St. Louis, Mo.) and lipopolysaccharide from Salmonella minnesota (LPS) (Invivogen, San Diego, Calif.) to induce the expression of indoleamine 2, 3-dioxygenase (IDO1). Compounds with IDO1 inhibitory properties decreased the amount of kynurenine produced by the cells via the tryptophan catabolic pathway. Cellular toxicity due to the effect of compound treatment was measured using CellTiter-Glo® reagent (CTG) (Promega Corporation, Madison, Wis.), which is based on luminescent detection of ATP, an indicator of metabolically active cells.

In preparation for the assays, test compounds were serially diluted 3-fold in DMSO from a typical top concentration of 5 mM and plated at 0.5 μL in 384-well, polystyrene, clear bottom, tissue culture treated plates with lids (Greiner Bio-One, Kremsmunster, Austria) to generate 11-point dose response curves. Low control wells (0% kynurenine or 100% cytotoxicity) contained either 0.5 μL of DMSO in the presence of unstimulated (-IFN-γ/-LPS) PBMCs for the mass spectrometry assay or 0.5 μL of DMSO in the absence of cells for the cytotoxicity assay, and high control wells (100% kynurenine or 0% cytotoxicity) contained 0.5 μL of DMSO in the presence of stimulated (+IFN-γ/+LPS) PBMCs for both the mass spectrometry and cytotoxicity assays.

Frozen stocks of PBMCs were washed and recovered in RPMI 1640 medium (Thermo Fisher Scientific, Inc., Waltham, Mass.) supplemented with 10% v/v heat-inactivated fetal bovine serum (FBS) (Thermo Fisher Scientific, Inc., Waltham, Mass.), and 1× penicillin-streptomycin antibiotic solution (Thermo Fisher Scientific, Inc., Waltham, Mass.). The cells were diluted to 1,000,000 cells/mL in the supplemented RPMI 1640 medium. 50 μL of either the cell suspension, for the mass spectrometry assay, or medium alone, for the cytotoxicity assay, were added to the low control wells, on the previously prepared 384-well compound plates, resulting in 50,000 cells/well or 0 cells/well respectively. IFN-γ and LPS were added to the remaining cell suspension at final concentrations of 100 ng/ml and 50 ng/ml respectively, and 50 μL of the stimulated cells were added to all remaining wells on the 384-well compound plates. The plates, with lids, were then placed in a 37° C., 5% CO₂ humidified incubator for 2 days.

Following incubation, the 384-well plates were removed from the incubator and allowed to equilibrate to room temperature for 30 minutes. For the cytotoxicity assay, CellTiter-Glo® was prepared according to the manufacturer's instructions, and 40 μL were added to each plate well. After a twenty minute incubation at room temperature, luminescence was read on an EnVision® Multilabel Reader (PerkinElmer Inc., Waltham, Mass.). For the mass spectrometry assay, 10 μL of supernatant from each well of the compound-treated plates were added to 40 μL of acetonitrile, containing 10 μM of an internal standard for normalization, in 384-well, polypropylene, V-bottom plates (Greiner Bio-One, Kremsmunster, Austria) to extract the organic analytes. Following centrifugation at 2000 rpm for 10 minutes, 10 μL from each well of the acetonitrile extraction plates were added to 90 μL of sterile, distilled H₂O in 384-well, polypropylene, V-bottom plates for analysis of kynurenine and the internal standard on the RapidFire 300 (Agilent Technologies, Santa Clara, Calif.) and 4000 QTRAP MS (SCIEX, Framingham, Mass.). MS data were integrated using Agilent Technologies' RapidFire Integrator software, and data were normalized for analysis as a ratio of kynurenine to the internal standard.

The data for dose responses in the mass spectrometry assay were plotted as % IDO1 inhibition versus compound concentration following normalization using the formula 100−(100*((U−C2)/(C1−C2))), where U was the unknown value, C1 was the average of the high (100% kynurenine; 0% inhibition) control wells and C2 was the average of the low (0% kynurenine; 100% inhibition) control wells. The data for dose responses in the cytotoxicity assay were plotted as % cytotoxicity versus compound concentration following normalization using the formula 100−(100*((U−C2)/(C1−C2))), where U was the unknown value, C1 was the average of the high (0% cytotoxicity) control wells and C2 was the average of the low (100% cytotoxicity) control wells.

Curve fitting was performed with the equation y=A+((B−A)/(1+(10^{x}/10^{c})D)), where A was the minimum response, B was the maximum response, C was the log(XC₅₀) and D was the Hill slope. The results for each test compound were recorded as pIC50 values for the mass spectrometry assay and as pCC50 values for the cytoxicity assay (-C in the above equation).

TABLE 1

| | IDO1 PBMC |
|---|---|
| example | pIC₅₀ |
| 1 | 8.1 |
| 2 | 8.1 |
| 3 | 8.0 |
| 4 | 8.4 |
| 5 | 8.1 |
| 6 | 8.0 |
| 7 | 8.2 |
| 8 | 8.2 |
| 9 | 8.3 |
| 10 | 8.3 |
| 11 | 8.3 |
| 12 | 8.5 |
| 13 | 8.1 |
| 14 | 8.4 |
| 15 | 8.4 |
| 16 | 9.1 |
| 17 | 8.1 |
| 18 | 8.2 |
| 19 | 8.4 |
| 20 | 8.7 |
| 21 | 8.8 |
| 22 | 8.7 |
| 23 | 8.4 |
| 24 | 8.5 |
| 25 | 8.5 |
| 26 | 8.1 |
| 27 | 8.0 |
| 28 | 8.1 |
| 29 | 8.8 |
| 30 | 8.0 |
| 31 | 8.1 |
| 32 | 8.0 |
| 33 | 8.1 |
| 34 | 8.4 |
| 35 | 8.2 |
| 36 | 8.8 |
| 37 | 8.6 |
| 38 | 8.3 |
| 39 | 8.3 |
| 40 | 8.4 |
| 41 | 8.4 |
| 42 | 8.3 |
| 43 | 9.0 |
| 44 | 8.5 |
| 45 | 8.5 |
| 46 | 8.4 |
| 47 | 8.4 |
| 48 | 8.4 |
| 49 | 8.2 |
| 50 | 8.2 |
| 51 | 8.1 |
| 52 | 8.0 |
| 53 | 8.5 |
| 54 | 7.9 |
| 55 | 7.9 |
| 56 | 7.9 |
| 57 | 7.9 |
| 58 | 7.9 |
| 59 | 7.9 |
| 60 | 7.9 |
| 61 | 7.9 |
| 62 | 7.9 |
| 63 | 7.9 |
| 64 | 7.9 |
| 65 | 7.9 |
| 66 | 7.9 |
| 67 | 7.9 |
| 68 | 7.8 |
| 69 | 7.8 |
| 70 | 7.8 |
| 71 | 7.8 |
| 72 | 7.8 |
| 73 | 7.8 |
| 74 | 7.8 |
| 75 | 7.8 |
| 76 | 7.8 |
| 77 | 7.8 |
| 78 | 7.8 |
| 79 | 7.8 |
| 80 | 7.8 |
| 81 | 7.8 |
| 82 | 7.8 |
| 83 | 7.8 |
| 84 | 7.7 |
| 85 | 7.7 |
| 86 | 7.7 |
| 87 | 7.7 |
| 88 | 7.7 |
| 89 | 7.7 |
| 90 | 7.7 |
| 91 | 7.7 |
| 92 | 7.7 |
| 93 | 7.7 |
| 94 | 7.7 |
| 95 | 7.7 |
| 96 | 7.7 |
| 97 | 7.6 |
| 98 | 7.6 |
| 99 | 7.6 |
| 100 | 7.6 |
| 101 | 7.6 |
| 102 | 7.6 |
| 103 | 7.6 |
| 104 | 7.6 |
| 105 | 7.6 |
| 106 | 7.6 |
| 107 | 7.5 |
| 108 | 7.5 |
| 109 | 7.5 |
| 110 | 7.5 |
| 111 | 7.5 |
| 112 | 7.5 |
| 113 | 7.5 |
| 114 | 7.5 |
| 115 | 7.5 |
| 116 | 7.5 |
| 117 | 7.5 |
| 118 | 7.5 |
| 119 | 7.5 |
| 120 | 7.4 |
| 121 | 7.4 |
| 122 | 7.4 |
| 123 | 7.4 |
| 124 | 7.4 |
| 125 | 7.4 |
| 126 | 7.4 |
| 127 | 7.4 |
| 128 | 7.4 |
| 129 | 7.4 |
| 130 | 7.3 |
| 131 | 7.3 |
| 132 | 7.3 |
| 133 | 7.3 |
| 134 | 7.3 |
| 135 | 7.3 |
| 136 | 7.3 |
| 137 | 7.3 |
| 138 | 7.2 |
| 139 | 7.2 |
| 140 | 7.2 |
| 141 | 7.2 |
| 142 | 7.2 |
| 143 | 7.1 |
| 144 | 7.1 |
| 145 | 7.1 |
| 146 | 7.1 |
| 147 | 7.1 |
| 148 | 7.1 |
| 149 | 7.1 |
| 150 | 7.1 |
| 151 | 7.1 |
| 152 | 7.1 |
| 153 | 7.1 |
| 154 | 7.1 |
| 155 | 7.1 |
| 156 | 7.1 |
| 157 | 7.0 |
| 158 | 7.0 |
| 159 | 7.0 |
| 160 | 7.0 |
| 161 | 7.0 |
| 162 | 7.0 |
| 163 | 7.0 |
| 164 | 7.0 |
| 165 | 7.0 |
| 166 | 7.0 |
| 167 | 7.0 |
| 168 | 7.0 |
| 169 | 7.0 |
| 170 | 7.0 |
| 171 | 7.0 |
| 172 | 7.0 |
| 173 | 6.9 |
| 174 | 6.9 |
| 175 | 6.9 |
| 176 | 6.9 |
| 177 | 6.9 |
| 178 | 6.9 |
| 179 | 6.9 |
| 180 | 6.9 |
| 181 | 6.8 |
| 182 | 6.8 |
| 183 | 6.8 |
| 184 | 6.8 |
| 185 | 6.8 |
| 186 | 6.8 |
| 187 | 6.8 |
| 188 | 6.8 |
| 189 | 6.7 |
| 190 | 6.7 |
| 191 | 6.7 |
| 192 | 6.7 |
| 193 | 6.7 |
| 194 | 6.6 |
| 195 | 6.6 |
| 196 | 6.6 |
| 197 | 6.6 |
| 198 | 6.6 |
| 199 | 6.5 |
| 200 | 6.5 |
| 201 | 6.5 |
| 202 | 6.5 |
| 203 | 6.5 |
| 204 | 6.5 |
| 205 | 6.5 |
| 206 | 6.5 |
| 207 | 6.4 |
| 208 | 6.4 |
| 209 | 6.4 |
| 210 | 6.4 |
| 211 | 6.4 |
| 212 | 6.4 |
| 213 | 6.4 |
| 214 | 6.4 |
| 215 | 6.4 |
| 216 | 6.3 |
| 217 | 6.3 |
| 218 | 6.3 |
| 219 | 6.3 |
| 220 | 6.3 |
| 221 | 6.3 |
| 222 | 6.3 |
| 223 | 6.3 |
| 224 | 6.3 |
| 225 | 6.3 |
| 226 | 6.2 |
| 227 | 6.2 |
| 228 | 6.2 |
| 229 | 6.2 |
| 230 | 6.2 |
| 231 | 6.2 |
| 232 | 6.1 |
| 233 | 6.1 |
| 234 | 6.1 |
| 235 | 6.1 |
| 236 | 6.0 |
| 237 | 6.0 |
| 238 | 6.0 |
| 239 | 6.0 |
| 240 | 6.0 |
| 241 | 6.0 |
| 242 | 6.0 |
| 243 | 6.0 |
| 244 | 6.0 |
| 245 | 6.0 |

1. A compound of Formula I of Formula I 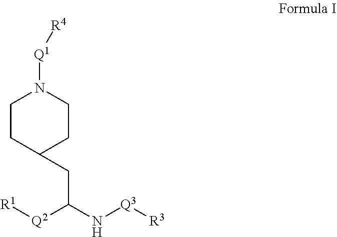
or a pharmaceutically acceptable salt thereof wherein:
Q¹ is C(O)O, C(O)CF₂, C(O)NH, SO₂, C(O), or a bond (i.e. is absent);
Q² is C₁₋₄alkyl, C₁₋₃alkyNHC₁₋₃alkyl, or a bond (i.e. is absent);
Q³ is C(O), C(O)NH, or a bond (i.e. is absent);
R¹ is C₁₋₆alkyl, C₂₋₄alkenyl, C₃₋₇cycloalkyl, C₅₋₉aryl, C₅₋₉heteroaryl, 5 to 9 membered heterocycle; wherein R¹ is optionally substituted with a substituent selected from C₁₋₆alkyl, OC₁₋₃alkyl, OC₃₋₆cycloalkyl, oxo, and N(R²)₂ wherein each R² is independently H, C₁₋₆alkyl, C₃₋₇cycloalkyl, C₁₋₃alkylOC₁₋₃alkyl, —OC₁₋₃alkylOC₁₋₃alkyl C₃₋₆cycloalkyl, —CH₂phenyl, or OCH₂phenyl;
R³ is C₅₋₉aryl, C₅₋₉heteroaryl, C₁₋₆alkyl, C₃₋₆cycloalkyl, C₇₋₁₀bicycloalkyl, wherein R³ is optionally substituted with 1 or 2 substituents selected from halogen, C₁₋₆alkyl, C₁₋₃fluoroalkyl, C₃₋₆cycloalkyl, OC₁₋₃alkyl, SC₁₋₃alkyl, C₂₋₄alkenyl, C₂₋₄alkynyl, OC₂₋₄alkyny, phenyl, CN;
R⁴ is C₅₋₉aryl, C₁₋₆alkyl, C₁₋₃fluoroalkyl, C₃₋₆cycloalkyl, C₂₋₄alkenyl, C₂₋₄alkynyl, or C₃₋₆ether;
and wherein each aryl and heteroaryl includes bicycles and wherein each heteroaryl, and heterocycle contains from 1 to 3 heteroatoms selected from O, N, and S.

2. A compound or salt according to claim 1 wherein Q¹ is C(O)O, C(O)CF₂, C(O)NH, SO₂, or C(O).

3. A compound or salt according to claim 1 wherein Q² is absent.

4. A compound or salt according to claim 1 wherein Q³ is C(O).

5. A compound or salt according to claim 1 wherein R¹ is phenyl, a pyridine, an oxadiazole, oxo substituted oxadiazole, C₁₋₆alkyl, C₃₋₇cycloalkyl, C₂₋₄alkenyl, a 5 or 6-membered heterocycle containing one or two heteroatoms selected from O and N, wherein R¹ is optionally substituted with a substituent selected from C₁₋₆alkyl, OC₁₋₃alkyl, OC₃₋₆cycloalkyl, and N(R²)₂ wherein each R² is independently H, C₁₋₆alkyl, C₃₋₇cycloalkyl C₁₋₃alkylOC₁₋₃alkyl, —OC₁₋₃alkylOC₁₋₃alkyl C₃₋₆cycloalkyl, —CH₂phenyl, or OCH₂phenyl.

6. A compound or salt according to claim 5 wherein R¹ is phenyl, a pyridine, an oxadiazole, C₁₋₆alkyl, C₃₋₇cycloalkyl, or C₂₋₄alkylenyl, wherein R¹ is optionally substituted with a substituent selected from C₁₋₆alkyl, OC₁₋₃alkyl, and N(R²)₂ wherein each R² is independently C₁₋₆alkyl, or C₃₋₆cycloalkyl.

7. A compound or salt according to claim 1 wherein R³ is thiophene, phenyl, pyridyl, benzoxazole, oxazole, C₁₋₆alkyl, C₃₋₆cycloalkyl, or C₇₋₁₀bicycloalkyl, wherein R³ is optionally substituted with 1 or 2 substituents selected from halogen, C₁₋₃alkyl, C₁₋₃fluoroalkyl, OC₁₋₃alkyl, SC₁₋₃alkyl, C₂₋₄alkenyl, C₂₋₄alkynyl, and OC₂₋₄alkynyl.

8. A compound or salt according to claim 7 wherein R³ is thiophene or phenyl optionally substituted with 1 or 2 substituents selected from halogen, C₁₋₃alkyl, and C₂₋₃alkynyl.

9. A compound or salt according to claim 1 wherein R⁴ is phenyl, C₁₋₆alkyl, C₁₋₃fluoroalkyl, C₃₋₆cycloalkyl, C₂₋₄alkynyl, or C₃₋₆ether.

10. A compound or salt according to claim 9 wherein R⁴ is C₁₋₆alkyl.

11. A compound or salt according to claim 1 wherein Q¹ is C(O)O, C(O)CF₂, C(O)NH, SO₂, or C(O); Q² is absent Q³ is C(O); R¹ is phenyl, a pyridine, an oxadiazole, oxo substituted oxadiazole, C₁₋₆alkyl, C₃₋₇cycloalkyl, C₂₋₄alkenyl, or a 5 or 6-membered heterocycle containing one or two heteroatoms selected from O and N, wherein R is optionally substituted with a substituent selected from C₁₋₆alkyl, OC₁₋₃alkyl, OC₃₋₆cycloalkyl, and N(R²)₂ wherein each R² is independently H, C₁₋₆alkyl, C₃₋₇cycloalkyl C₁₋₃alkylOC₁₋₃alkyl, —OC₁₋₃alkylOC₁₋₃alkyl C₃₋₆cycloalkyl, —CH₂phenyl, or OCH₂phenyl; R³ is thiophene, phenyl, pyridyl, benzoxazole, oxazole, C₁₋₆alkyl, C₃₋₆cycloalkyl, or C₇₋₁₀bicycloalkyl, wherein R³ is optionally substituted with 1 or 2 substituents selected from halogen, C₁₋₃alkyl, C₁₋₃fluoroalkyl, OC₁₋₃alkyl, SC₁₋₃alkyl, C₂₋₄alkenyl, C₂₋₄alkynyl, and OC₂₋₄alkynyl; and R⁴ is phenyl, C₁₋₆alkyl, C₁₋₃fluoroalkyl, C₃₋₆cycloalkyl, C₂₋₄alkynyl, or C₃₋₆ether.

12. A pharmaceutical composition comprising a compound or salt according to claim 1.

13. A method of treating a disease or condition that would benefit from inhibition of IDO1 comprising the step of administration of a composition according to claim 12.

14. The method of claim 13 wherein in said disease or condition, biomarkers of IDO activity are elevated.

15. The method of claim 13 wherein said biomarkers are plasma kynurenine or the plasma kynurenine/tryptophan ratio.

16. The method of claim 13 wherein said disease or condition is chronic viral infection; chronic bacterial infections; cancer; sepsis; or a neurological disorder.

17. The method of claim 13 wherein said chronic viral infections are those involving HIV, HBV, or HCV; said chronic bacterial infections are tuberculosis or prosthetic joint infection; and said neurological disorders are major depressive disorder, Huntington's disease, or Parkinson's disease.

18. The method of claim 17 wherein said disease or condition is inflammation associated with HIV infection; chronic viral infections involving hepatitis B virus or hepatitis C virus; cancer; or sepsis.

19-20. (canceled)
